(19) United States
(12) Patent Application Publication
(10) Pub. No.: US 2009/0155903 A1
(43) Pub. Date: Jun. 18, 2009

(54) PHARMACEUTICAL COMPOSITION AND METHOD

(75) Inventors: Rachel M. Slade, Salt Lake City, UT (US); Warren S. Weiner, Salt Lake City, UT (US); Eric Delmar, Salt Lake City, UT (US); Yevgeniya Klimova, Murray, UT (US); Richard Trovato, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 10/593,180

(22) PCT Filed: Mar. 21, 2005
(86) PCT No.: PCT/US05/09595

§ 371 Date: Nov. 14, 2008

Related U.S. Application Data
(60) Provisional application No. 60/554,571, filed on Mar. 19, 2004.
(60) Provisional application No. 60/590,259, filed on Jul. 22, 2004.

(51) Int. Cl. C12N 5/06; C07D 209/12; C07D 401/04

(52) U.S. Cl. 435/375; 548/500; 546/277.4

(57) ABSTRACT

The invention provides compounds, pharmaceutical compositions and methods for the therapeutic treatment and prevention of neurodegenerative disorder and other Aβ₄₂-related diseases and disorders.

### CROSS-REFERENCE TO RELATED APPLICATIONS

This application claims priority under 35 U.S.C. § 119(e) to U.S. provisional application Ser. No. 60/590,259 filed Jul. 22, 2004 and U.S. provisional application Ser. No. 60/554,571, filed Mar. 19, 2004 which are both hereby incorporated by reference in its entirety.

### FIELD OF THE INVENTION

The invention generally relates to compounds, pharmaceutical compositions and methods of use thereof, and particularly to compounds and compositions useful in treating and preventing diseases and disorders amenable to lowering cellular Aβ₄₂ production and/or secretion, including Alzheimer's disease, mild cognitive impairment and others.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60 with the risk increasing with age. Younger people can also get AD, but it is much less common. It is estimated that 3 percent of men and women ages 65 to 74 have AD. Almost half of those ages 85 and older may have the disease. AD is not a normal part of aging. Alzheimer's disease is a complex disease that can be caused by genetic and environmental factors. In the United States alone, four million adults suffer from Alzheimer's disease (AD). Not only does Alzheimer's disease significantly impact the lives of countless families today, it threatens to become even more of a problem as the baby boom generation matures. The economic burden of AD in the United States is estimated to cost over $100 billion a year and the average lifetime cost per patient is estimated to be $174,000. Unfortunately, there is no cure available for AD.

In 1906, Dr. Alois Alzheimer, noticed changes in the brain tissue of a woman who had died of an unusual mental illness. In her brain tissue, he found abnormal clumps (now known as amyloid plaques) and tangled bundles of fibers (now known as neurofibrillary tangles) which, today, are considered the pathological hallmarks of AD. Other brain changes in people with AD have been discovered. For example, with AD, there is a loss of nerve cells in areas of the brain that are vital to memory and other mental abilities. Scientists have also found that there are lower levels of chemicals in the brain that carry complex messages back and forth between nerve cells. AD may disrupt normal thinking and memory by blocking these messages between nerve cells.

Plaques and tangles are found in the same brain regions that are affected by neuronal and synaptic loss. Neuronal and synaptic loss is universally recognized as the primary cause in decline of cognitive function. The number of tangles is more highly correlated with the cognitive decline than amyloid load in patients with AD (Albert Proc. Natl. Acad. Sci. U.S.A. 93:13547-13551 (1996)). The cellular, biochemical, and molecular events responsible for neuronal and synaptic loss in AD are not known. A number of studies have demonstrated that amyloid can be directly toxic to neurons (Iversen et al. Biochem. J. 311:1-16 (1995); Weiss et al. J. Neurochem. 62:372-375 (1994); Lorenzo et al. Ann. N.Y. Acad. Sci. 777:89-95 (1996); Storey et al. Neuropathol. Appl. Neurobiol. 2:81-97 (1999), resulting in behavioral impairment. The toxicity of amyloid or tangles is potentially aggravated by activation of the complement cascade (Rogers et al. Proc. Natl. Acad. Sci. U.S.A. 21:10016-10020 (1992); Rozemuller et al. Res. Immunol. 6:646-9 (1992); Rogers et al. Res. Immunol. 6:624-30 (1992); Webster et al. J. Neurochem. 69(1):388-98 (1997)). This suggests involvement of an inflammatory process in AD and neuronal death seen in AD (Fagarasan et al. Brain Res. 723(1-2):231-4. (1996); Kalaria et al. Neurodegeneration 5(4):497-503 (1996); Kalaria et al. Neurobiol Aging. 17(5):687-93 (1996); Farlow Am. J. Health Syst. Pharm. 55 Suppl. 2:S5-10 (1998).

Evidence that amyloid β protein (Aβ deposition causes some forms of AD was provided by genetic and molecular studies of some familial forms of AD (FAD). (See, e.g., Ii Drugs Aging 7(2):97-109 (1995); Hardy Proc. Natl. Acad. Sci. U.S.A. 94(6):2095-7 (1997); Selkoe J. Biol. Chem. 271(31):18295-8 (1996)). The amyloid plaque buildup in AD patients suggests that abnormal processing of Aβ may be a cause of AD. Aβ is a peptide of 39 to 42 amino acids and forms the core of senile plaques observed in all Alzheimer cases. If abnormal processing is the primary cause of AD, then familial Alzheimer's disease (FAD) mutations that are linked (genetically) to FAD may induce changes that, in one way or another, foster Aβ deposition. There are 3 FAD genes known so far (Hardy et al. Science 282:1075-9 (1998); Ray et al. (1998)). Mutations in these FAD genes can result in increased Aβ deposition.

The first of the 3 FAD genes codes for the Aβ precursor, amyloid precursor protein (APP) (Selkoe J. Biol. Chem. 271(31):18295-8 (1996)). Mutations in the APP gene are very rare, but all of them cause AD with 100% penetrance and result in elevated production of either total Aβ or Aβ42, both in model transfected cells and transgenic animals. The other two FAD genes code for presenilin 1 and 2 (PS1, PS2) (Hardy Proc. Natl. Acad. Sci. U.S.A. 94(6):2095-7 (1997)). The presenilins contain 8 transmembrane domains and several lines of evidence suggest that they are involved in intracellular protein trafficking. Other studies suggest that the presenilins function as proteases. Mutations in the presenilin genes are more common than in the APP gene, and all of them also cause FAD with 100% penetrance. Similar to APP mutants, studies have demonstrated that PS1 and PS2 mutations shift APP metabolism, resulting in elevated Aβ42 production (in vitro and in vivo).

Cyclooxygenases (COX) are major Alzheimer's disease drug targets due to the epidemiological association of NSAID use, whose primary target are cycloxygenases, with a reduced risk of developing Alzheimer's disease (see, e.g., Hoozemans et al. Curr. Drug Targets 4(6):461-8 (2003) and Pasinetti et al. J. Neurosci. Res. 54(1):1-6 (1998)). The epidemiological studies have indicated that chronic NSAID use appears to reduce the risk of acquiring Alzheimer's disease and/or delay the onset of the disease (see e.g., McGeer et al. Neurology 47(2):425-432 (1996); and Etminan et al. BMJ. 327(7407):128 (2003)). COX-2 selective inhibitors are attractive candidates for long-term drug use since they do not inhibit COX-1 and appear to be less toxic. In support of COX-2 as a target for the treatment for AD, a recent study was published reporting that in mouse models of AD, COX-2 overexpression was related to the neuropathology of AD (Xiang et al. Neurobiol. Aging 23:327-34 (2002)). However, recent clinical trials of specific NSAIDs have called into question the hypothesis the hypothesis that anti-inflammatory drugs are useful for the treatment or prevention of Alzheimer's disease. It was reported that rofecoxib, a COX-2 selective NSAID, at 25 mg daily, failed to show efficacy for treating AD. Naproxen, another NSAID, in the same trial failed to show efficacy in Alzheimer's treatment. See Aisen et al. JAMA 289:2819-26 (2003) and Reines et al. Neurology 62(1):66-71 (2004). These authors concluded that the results with naproxen and rofecoxib do not support the use of NSAIDs for the treatment of AD. Celecoxib, a COX-2-selective NSAID, failed to show efficacy in several recent clinical trials for the treatment of AD. See Jhee et al., “A Double-Blind, Placebo-Controlled Pharmacokinetic (PK), Pharmacodynamic (PD) and Safety Study of Celecoxib Treatment for Four Weeks in Patients with Alzheimer's Disease (AD),” Abstract from 7th International Geneva/Springfield Symposium on Advances in Alzheimer's Therapy (2002); also published in Clinical Research and Regulatory Affairs 21(1): 49-66 (2004)) and Sainati et al. (Abstract from 6th International Stockholm/Springfield Symposium on Advances on Alzheimer's Therapy, Abstract Book 2000; 180). Conversely, it was reported recently that rofecoxib provides neuroprotection in an in vivo Alzheimer's disease excitotoxic model system (Scali et al. Neuroscience 117:909-919 (2003). However, rofecoxib, in a large prevention clinical trial, failed to prevent the development of Alzheimer's disease in patients having mild cognitive impairment. In fact, the results of this trial showed that 6.4% of patients taking rofecoxib developed AD as compared to 4.5% for those taking placebo (see e.g., Visser et al., abstract from Annual meeting of the American College of Neuropsychopharmacology San Juan, Puerto Rico, 2003; and Landers, Wall Street Journal 10 Dec. 2003). Thus, clinical trials have indicated that NSAIDs, as a general class of drugs, are not likely to be useful for treating and/or preventing Alzheimer's disease.

Of the five drugs currently being used in the US for the treatment of AD, four of them—tacrine (Cognex®), donepezil (Aricept®), rivastigmine (Exelon®), and galantamine (Reminyl®)—are inhibitors of acetylcholinesterase. Another drug, memantine, was recently approved for treating moderate-to-severe AD. More recently it was reported that memantine showed efficacy in treating mild-to-moderate AD. Memantine is a NMDA receptor antagonist.

The drugs currently used for treating AD, including memantine and the acetylcholine esterase inhibitors, are marginally efficacious and have undesirable side-effects. Thus, there is a large unmet need for better and safer drugs.

### BRIEF SUMMARY OF THE INVENTION

In general, the invention relates to the use of compounds of Formula I-Va, to reduce Aβ₄₂ in mammalian cells and to treat diseases and disorders amenable to reduction of cellular Aβ₄₂ production or secretion, such as neurodegenerative disorders (dementia, Alzheimer's disease, MCI, Parkinson's disease, Down's syndrome, etc.), inclusion body myositis, and tauopathies (corticobasal degeneration, and progressive supranuclear palsy). The invention provides compounds of Formula I-Va, pharmaceutically acceptable salts thereof, and pharmaceutical compositions having such compounds.

Compounds of the invention include those of Formula I below:

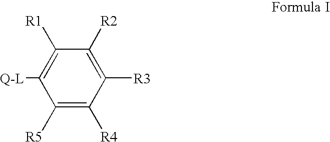

wherein one or more of R1-R5 is selected from the group consisting of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, L-C(═O)CH₂SH, -L-C(═O)NHCN, -L-sulfo, -L-(2,6 difluorophenol), -L-phosphono, and -L-tetrazolyl, and the others of R1-R5, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

L can be saturated, partially saturated, or unsaturated, and is independently selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙO(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl; and

Q is selected from the group consisting of optionally substituted aryl, optionally substituted heterocycle, optionally substituted heteroaryl, and optionally substituted cycloalkyl.

Compounds of the invention include those of Formula IIa and IIb below:

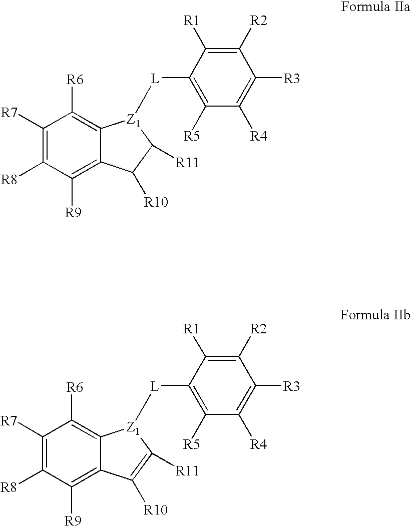

wherein one or more of R1-R10 is selected from the group consisting of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, L-C(═O)CH₂SH, -L-C(═O)NHCN, -L-sulfo, -L-(2,6 difluorophenol), -L-phosphono, and -L-tetrazolyl, and the others of R1-R10, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Z is a carbon atom or a nitrogen atom; and

L can be saturated, partially saturated, or unsaturated, and is selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙO(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl.

Compounds of the invention include those of Formula III below:

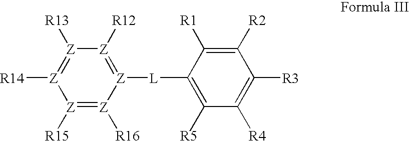

wherein one or more of R1-R5 is selected from the group consisting of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, L-C(═O)CH₂SH, -L-C(═O)NHCN, -L-sulfo, -L-(2,6 difluorophenol), -L-phosphono, and -L-tetrazolyl, and the others of R1-R5, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R12-R16, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

L can be saturated, partially saturated, or unsaturated, and is selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙO(CH₂)—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl; and

each Z is independently selected from the group consisting of a carbon atom, and a nitrogen atom.

Compounds of the invention include those of Formula IV below:

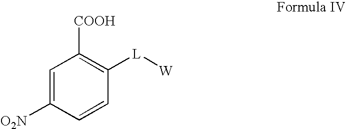

L can be saturated, partially saturated, or unsaturated, and is selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙO(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl; and

W is selected from the group consisting of optionally substituted cycloalkyl, optionally substituted aril, optionally substituted heterocycle, and optionally substituted heteroaryl.

Optionally substituted, when used herein without reference to further definition, refers to a substituent selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

Furthermore, the invention provides derivatives or analog of the compounds defined in aspects one through ten of the invention, where the derivative or analog is selected from an ester, an amide, a carbamate, a urea, an amadine, or a combination thereof. Methods of generating an ester, an amide, a carbamate, a urea, an amadine, or a combination thereof of the compounds of the invention are known to an ordinary artisan skilled in organic chemical synthesis.

In another aspect, the invention provides a method of treating a neurodegenerative disorder, by identifying a patient in need of such treatment, and administering to the patient a therapeutically effective amount of a pharmaceutical composition having one or more compounds of Formula I-Va. Administration of a compound of Formula I-Va for at least 4 weeks, preferably at least 4 months, and more desirably at least 8 months, can provide an improvement or lessening in decline of cognitive function as characterized by cognition tests, biochemical disease marker progression, and/or plaque pathology. Cognition tests are those which are capable of measuring cognitive decline in a patient or group of patients. Examples of such cognition tests include the ADAS-cog (Alzheimer's Disease Assessment Scale, cognitive subscale) NPI (Neuropsychiatric Inventory), ADCS-ADL (Alzheimer's Disease Cooperative Study-Activities of Daily Living), CIBIC-plus (Clinician Interview Based Impression of Change), and CDR sum of boxes (Clinical Dementia Rating). It is preferred that the lessening in decline in cognitive function is at least 25% as compared to individuals treated with placebo, more preferably at least 40%, and even more desirably at least 60%. For example, an individual treated with placebo having probable mild-to-moderate Alzheimer's disease is expected to score approximately 5.5 points lower on the ADAS-cog test after a specified period of time of treatment (e.g., 1 year) whereas an individual treated with the composition of this aspect of the invention for the same period of time will score approximately 2.2 points lower on the ADAS-cog scale with a 60% decrease in decline or 3.3 points lower with a 40% decrease in decline in cognitive function when treated with the composition for the same specified period of time. Desirably, the oral dose is provided in capsule or tablet form. The pharmaceutical composition for use in the invention is formulated with one or more pharmaceutically acceptable excipients, salts, or carriers. The pharmaceutical composition for use in the invention is delivered orally, preferably in a tablet or capsule dosage form.

In one aspect, the invention provides a method for prophylaxis against a neurodegenerative disorder, by identifying a patient in need of or desiring such treatment, and administering to the patient a prophylactically effective amount of a pharmaceutical composition having one or more compounds of Formula I-Va. Administration of a compound of Formula I-Va for at least 4 weeks, preferably at least 4 months, and more desirably at least 8 months, can delay the onset of the neurodegenerative disorder or slow the rate of onset of symptoms of the disorder. Patients having a predisposition to a neurodegenerative disorder or suspected of needing prophylaxis can be identified by any method known to the skilled artisan for diagnosis such neurodegenerative disorders.

In another aspect, the invention provides a method of treating a disease characterized by abnormal amyloid precursor protein processing by (1) identifying a patient in need of such treatment, and (2) administering to the patient a therapeutically effective amount of a pharmaceutical composition having one or more compounds of Formula I-Va. Oral administration of the pharmaceutical composition for use in the method of this aspect the invention for at least 4 weeks, preferably at least 4 months, and more desirably at least 8 months, provides an improvement or lessening in decline of cognitive function as characterized by cognition tests, biochemical disease marker progression, and/or plaque pathology. Examples of biochemical disease markers include, for example, amyloid beta peptide (Aβ), Aβ42, and tau. It is preferred that the lessening in decline in biochemical disease marker progression is at least 10% as compared to individuals treated with placebo, more preferably at least 20%, and more desirably at least 40%. It is preferred that the lessening in decline in cognitive function is at least 25% as compared to individuals treated with placebo, more preferably at least 40%, and even more desirably at least 60%. Desirably, the composition is provided as an oral dose, preferably in capsule or tablet form.

In an aspect, the invention provides a method of prophylaxis or delaying the onset of a disease (or one or more symptoms thereof) characterized by abnormal amyloid precursor protein processing, by identifying a patient in need of such treatment and administering to the patient a prophylactically effective amount of a pharmaceutical composition having one or more compounds of Formula I-Va. Oral administration of the pharmaceutical composition for use in the method of this aspect the invention for at least 4 weeks, preferably at least 4 months, and more desirably at least 8 months, prevents or delays the onset of the disease (or symptoms thereof) characterized by abnormal amyloid precursor protein processing.

In another aspect, the invention provides a method of treating Alzheimer's disease comprising administering to a patient in need of such treatment, a pharmaceutical composition having one or more compounds of Formula I-Va. Oral administration of the pharmaceutical composition for use in the method of this aspect of the invention for at least 4 weeks, preferably at least 4 months, and more desirably at least 8 months, provides an improvement or lessening in decline of cognitive function as characterized by cognition tests, biochemical disease marker progression, and/or plaque pathology. Desirably, the oral dose is provided in capsule or tablet form. According to this aspect of the invention, a patient in need of treatment is administered an Alzheimer's disease treating effective amount of a pharmaceutical composition having one or more compounds of Formula I-Va and one or more pharmaceutically acceptable salts, excipients and carriers. The method of this aspect of the invention involves identifying an individual likely to have mild-to-moderate Alzheimer's disease. An individual having probable mild-to-moderate Alzheimer's disease can be diagnosed by any method available to the ordinary artisan skilled in such diagnoses. For example, diagnosis can be according to DSM IV (TR) and/or meets NINCDS-ADRDA criteria for probable AD. According to this aspect of the invention, individuals with probable mild-to-moderate AD take an oral dose of a pharmaceutical composition for a specified period of time. Individuals undergoing such treatment are likely to see an improvement or lessening in decline of cognitive function, an improvement or lessening in decline in biochemical disease marker progression, and/or an improvement or lessening decline in plaque pathology. A lessening in decline in cognitive function can be assessed using a test of cognitive function like the ADAS-cog. For example, an individual treated with placebo having probable mild-to-moderate Alzheimer's disease is expected to score approximately 5.5 points lower on the ADAS-cog test after a specified period of time of treatment (e.g., 1 year) whereas an individual treated with the composition of this aspect of the invention for the same period of time will score approximately 2.2 points lower on the ADAS-cog scale with a 60% decrease in decline or 3.3 points lower with a 40% decrease in decline in cognitive function when treated with the composition for the same specified period of time. In a related aspect, the method involves identifying a patient having moderate-to-severe AD and administering to the patient an Alzheimer's disease treating effective amount of a compound of Formula I-Va.

In yet another aspect, the invention provides a method of preventing the onset of Alzheimer's disease comprising administering to a patient in need of or desiring such treatment, a pharmaceutical composition having one or more compounds of Formula I-Va. Oral administration of the pharmaceutical composition for use in the method of this aspect of the invention for at least 4 weeks, preferably at least 4 months, and more desirably at least 8 months, delays the onset of decline of cognitive function, biochemical disease marker progression, and/or plaque pathology. According to this embodiment, an individual desiring or needing preventative treatment against the onset of AD is administered a pharmaceutical composition having one or more compounds of Formula I-Va. Desirably, the oral dose is provided in capsule or tablet form. The preventive treatment is preferably maintained as long as the individual continues to desire or need the treatment. Individuals needing or desiring preventative treatment against AD can be those having risk factors for developing AD. For example, risk factors for developing AD can be genetic factors or environmental factors. In one embodiment, the risk factor is age. Genetic risk factors can be assessed in a variety of ways, such as ascertaining the family medical history of the individual, or performing a genetic test to identify genes that confer a predisposition for developing AD. Additionally, risk factors can be assessed by monitoring genetic and biochemical markers.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

In general, the invention relates to the use of pharmaceutical compositions having one or more compounds of Formula I-Va as the active ingredient, for reducing cellular Aβ₄₂ production and/or secretion, and treating neurodegenerative disorders and other Aβ₄₂-associated diseases and disorders. When the pharmaceutical composition is administered, according to the treatment regimens of the invention, to an individual desiring or needing such treatment, it provides an improvement or lessening in decline of cognitive function, biochemical disease marker progression, and/or plaque pathology associated with neurodegenerative disorders such as AD. The composition of the invention is formulated with one or more pharmaceutically acceptable excipients, salts, or carriers. The pharmaceutical compositions can be used in methods for reducing cellular Aβ₄₂ production and/or secretion, and for treating diseases and disorders characterized by abnormal amyloid precursor protein processing. Particularly, the compounds and pharmaceutical compositions containing the compounds are useful for treating neurodegenerative disorders (e.g., dementia, Alzheimer's disease, MCI, Parkinson's disease, Down's syndrome, etc.), inclusion body myositis, and tauopathies (corticobasal degeneration, and progressive supranuclear palsy). The invention therefore provides compounds of Formula I-Va and pharmaceutical composition having such compounds, for the treatment and prophylaxis of neurodegenerative disorders.

Compounds of the invention include those of Formula I below:

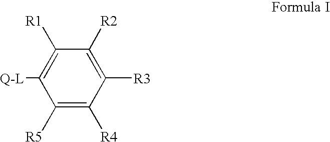

wherein one or more of R1-R5 is selected from the group consisting of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃ alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, L-C(═O)CH₂SH, -L-C(═O)NHCN, -L-sulfo, -L-(2,6 difluorophenol), -L-phosphono, and -L-tetrazolyl, and the others of R1-R5, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

L can be saturated, partially saturated, or unsaturated, and is independently selected from the group consisting of —(CH₂)ₙ—, —(CH₂)ₙC(═O)(CH₂)ₙ—, —(CH₂)ₙNH(CH₂)ₙ—, —(CH₂)ₙO(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl; and

Q is selected from the group consisting of optionally substituted aryl, optionally substituted heterocycle, optionally substituted heteroaryl, and optionally substituted cycloalkyl.

In one embodiment of the first aspect of the invention, one or more of R1-R5 in the compounds of Formula I, is selected from the group consisting of —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, —S(═O)₂N(C₁₋₃alkyl)₂, and the others of R1-R5, independent of one another, are selected from the group consisting of are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In another embodiment of this first aspect of the invention, L is a bond, one of R1-R5 is selected from the group consisting of —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others of R1-R5 independently are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

Compounds of the invention include those of Formula IIa and IIb below:

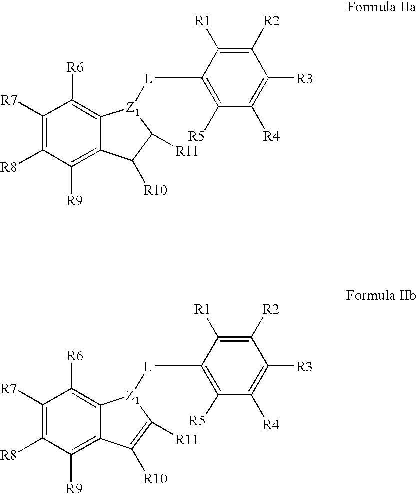

wherein one or more of R1-R10 is selected from the group consisting of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, L-C(═O)CH₂SH, -L-C(═O)NHCN, -L-sulfo, -L-(2,6 difluorophenol), -L-phosphono, and -L-tetrazolyl, and the others of R1-R10, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Z is a carbon atom or a nitrogen atom; and

L can be saturated, partially saturated, or unsaturated, and is selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙO(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl.

A preferred subset of compounds of Formula II include those where L is selected from the group consisting of —CH₂— and —C(═O)—; Z1 is nitrogen; R1 is hydro; R2 is selected from the group consisting of hydro, lower alkoxy, and halo (if halo then preferably chloro); R3 is selected from the group consisting of hydro, lower alkoxy, halo, haloalkyl, —CHF₂, —O—CF₃, —S—CF₃, and —CF₃; R4 is selected from the group consisting of hydro, lower alkoxy, and halo (if halo then preferably chloro); R5 is hydro; R6 is hydro; R7 is hydro; R8 is selected from hydro and —C(CH₃)₃; R10 is —CH₂C(═O)OH; R11 is —CH₃; with the provision that the compound is not indomethacin. Additionally, R2 and R3, or R3 and R4 can be taken together to form a 5 or 6 membered heterocyclic ring (preferably —O—CH₂—O— or —O—CF₂—O—). In a preferred subset of this subset, when R3 is not hydro, then R2 and R4 are halogen (preferably chloro). In another preferred subset of this subset, when R2 and R4 are both hydro, then R3 is selected from the group consisting of —O—CF₃, —S—CF₃, and —CF₃.

A preferred subset of compounds of Formula II include those where L is selected from the group consisting of —CH₂—, —CH₂—C(═O)—, —C(═O)—; Z1 is nitrogen; R1, R2, R4, and R5 are hydro; R3 is selected from the group consisting of halo (if halo preferably fluoro), haloalkyl, —CHF₂, —O—CF₃, —S—CF₃, and —CF₃; R6 is selected from the group consisting of hydro and —NO₂; R7 is selected from hydro, alkoxy, —O—CH₃, and lower alkyl; R8 is selected from the group consisting of hydro, alkoxy, —C(CH₃)₃, fluoro, chloro, —O—CH₃, haloalkyl, —CHF₂, —O—CF₃, —S—CF₃, —CF₃, —NO₂, —S(═O)₂—CH₃ and —O—CH₂-Aryl; R10 is hydro; and R11 is selected from the group consisting of —C(═O)OH and —CH₂C(═O)OH. Preferably, R11 is —C(═O)OH. Preferably there is a double bond between the carbons attached to R11 and R10 in the ring system containing Z1.

Another preferred subset of compounds of Formula II include those where L is selected from the group consisting of —CH₂— and —C(═O)—; Z1 is nitrogen; R1 is hydro; R2 is selected from the group consisting of hydro and halo (if halo then preferably chloro); R3 is selected from the group consisting of hydro; —O—CF₃, —S—CF₃, and —CF₃; R4 is selected from the group consisting of hydro and halo (if halo then preferably chloro); R5 is hydro; R6 is hydro; R7 is hydro; R8 is selected from hydro and —C(CH₃)₃; R10 is selected from the group consisting of —CH₂C(═O)OCH₂C(═O)OH and —CH₂C(═O)OH; R11 is —CH₃; with the provision that the compound is not indomethacin. Preferably, when R3 is not hydro then R2 and R4 are halogen (preferably chloro). Preferably, when R2 and R4 are both hydro then R3 is selected from the group consisting of —O—CF₃, —S—CF₃, and —CF₃.

In one embodiment, the compounds of Formula II have the following Formula IIc or pharmaceutically acceptable salts thereof:

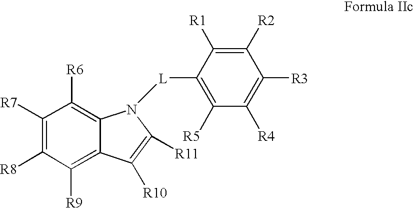

wherein L is —C(═O)—, —CH₂— or —CH(C₁₋₆ alkyl)-, and preferably —C(═O)— or —CH₂—;
R1, R2, R4, R5, R6, R7, and R9 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂; or C₁₋₆ alkyl-S— optionally substituted with 1, 2, 3, and 4-6 halo (e.g., F, Cl, Br, I), preferably —SCF₃;
R3 is selected from the group consisting of C₁₋₆ alkyl; C₁₋₆ haloalkyl; C₁₋₆ alkoxy or C₁₋₆ alkyl-thiol each optionally substituted with 1, 2, 3, or 4-6 halo; optionally R3 forms a 5 or 6-membered heterocycle with the adjacent R2 or R4 group; preferably R3 is —CHF₂, —CF₃, —OCF₃, —OCHF₂);
R8 is H; halo (e.g., F, Cl, Br, I); C₁₋₆ alkyl; C₁₋₆ haloalkyl (e.g., CHF₂, CF₃); C₂₋₆ alkoxy optionally substituted with 1, 2, 3, and 4-6 halo (e.g., F, Cl, Br, I), preferably ethoxy, propyloxy and isopropyloxy; C₂₋₆ alkyl-S— optionally substituted with 1, 2, 3, and 4-6 halo (e.g., F, Cl, Br, I); —S(O)₂—(C₁₋₆ alkyl); —NO₂; or optionally substituted benzyloxy;
R10 is selected from the group consisting of —R^{L}—C(═O)R₄₂, —R^{L}—C(═S)R₄₂,

—R^{L}—C(═O)SR₄₃, —R^{L}—C(═O)N(R₅₂)(R₅₃), —R^{L}-phosphono, and —R^{L}-tetrazolyl;
R11 is a C₁₋₃ alkyl (e.g., methyl, ethyl, propyl, isopropyl), preferably methyl;
R^{L} is selected from C₁₋₆ alkyl, C₂₋₆ alkenyl and C₂₋₆ alkynyl, preferably C₁ alkyl;
R₄₂ is selected from H, —OH, C₁₋₆ alkyl, C₂₋₆ alkenyl, C₂₋₆ alkynyl, C₁₋₆ alkoxy, C₂₋₆ alkenyloxy, C₂₋₆ alkynyloxy, and C₂₋₆ alkylthiol, wherein R₄₂ is optionally substituted with from one to three substituents independently selected from halo, N₃, nitro, hydroxy, thiol, CN and C₁₋₆ alkyl;
R₄₃ is H, C₁₋₆ alkyl, C₂₋₆ alkenyl or C₂₋₆ alkynyl, wherein R₄₃ is optionally substituted with from one to three substituents independently selected from halo, N₃, nitro, hydroxy, thiol, CN and C₁₋₆ alkyl; and
R₅₂ and R₅₃ are independently H, OH(R₅₂ and R₅₃ are not both OH), C₁₋₁₀ alkyl, C₂₋₁₀ alkenyl, C₂₋₁₀ to alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, or R₅₂ and R₅₃ together with the nitrogen atom to which they are both linked form a 3, 4, 5 or 6-membered heterocycle (e.g., piperidinyl, pyrrolidinyl, and morpholinyl), wherein R₅₂ and R₅₃ each is optionally substituted with 1-3 substituents wherein each substituent is independently halo, N₃, nitro, hydroxy, thiol, CN, C₁₋₆ alkyl, C₁₋₆ haloalkyl, C₁₋₆ alkoxy, —C(═O)N(R₅₄)(R₅₅), R₄₄C(═O)— or —N(R₅₄)(R₅₅), wherein R₅₄ and R₅₅ are independently H, OH or C₁₋₄ alkyl, and wherein R₄₄ is H or C₁₋₄ alkyl.

In a preferred embodiment of the compounds according to Formula IIc:
L is —C(═O)— or —CH₂—;
R1, R2, R4, R5, R6, R7, and R9 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂;
R3 is selected from the group consisting of —CHF₂, —CF₃, —OCF₃, —OCHF₂, and preferably —CF₃ or —OCF₃;
R8 is H; halo (e.g., F, Cl, Br, I); C₁₋₆ alkyl (e.g., preferably methyl, ethyl, propyl, isopropyl, or —C(CH₃)₃); C₁₋₆ haloalkyl (e.g., CHF₂, CF₃); or C₂₋₆ alkoxy optionally substituted with 1, 2, 3, and 4-6 halo (e.g., F, Cl, Br, I), preferably ethoxy, propyloxy and isopropyloxy;
R10 is —R^{L}—COOH, wherein R^{L} is selected from C₁₋₆ alkyl, C₂₋₆ alkenyl and C₂₋₆ alkynyl, preferably —CH₂—; and
R11 is a C₁₋₃ alkyl (e.g., methyl, ethyl, propyl, isopropyl), preferably methyl.

In another preferred embodiment of the compounds according Formula IIc,
L is —C(═O)—;
R1, R2, R4, R5, R6, R7, and R9 are independently H; halo (e.g., F, Cl, Br, I); C₁₋₃ alkyl; C₁₋₃ haloalkyl (e.g., CHF₂, CF₃); or C₁₋₃ alkoxy optionally substituted with 1, 2, 3, or 4 halo (e.g., F, Cl, Br, I), preferably —OCF₃, —OCHF₂; Preferably, R1, R2, R4, R5, R6, R7, and R9 are independently H or halo or methyl;
R3 is —OCF₃;
R8 is H; F, Cl or Br; C₁₋₆ alkyl (e.g., preferably methyl, ethyl, propyl, isopropyl, or —C(CH₃)₃); C₁₋₆haloalkyl (e.g., CHF₂, CF₃); or C₂₋₆ alkoxy optionally substituted with 1, 2, 3, and 4-6 halo (e.g., F, Cl, Br, I), preferably ethoxy, propyloxy and isopropyloxy;
For example, R8 may be selected from fluoro, chloro, bromo, ethyl, ethoxy;
R10 is —CH₂COOH; and
R11 is a C₁₋₃ alkyl (e.g., methyl, ethyl, propyl, isopropyl), preferably methyl.

In yet another embodiment of the compounds according Formula IIc,
L is —CH₂—;
R1, R2, R4, R5, R6, R7, and R9 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂;
R3 is —CF₃;
R8 is H; halo (e.g., F, Cl, Br, I); C₁₋₆ alkyl (e.g., preferably methyl, ethyl, propyl, isopropyl, or —C(CH₃)₃); C₁₋₆ haloalkyl (e.g., CHF₂, CF₃); or C₂₋₆ alkoxy optionally substituted with 1, 2, 3, and 4-6 halo (e.g., F, Cl, Br, I), preferably ethoxy, propyloxy and isopropyloxy; Preferably R8 is C₁₋₄ alkyl (e.g., methyl, ethyl, propyl, isopropyl, or —C(CH₃)₃);
R10 is —CH₂COOH; and
R11 is a C₁₋₃ alkyl (e.g., methyl, ethyl, propyl, isopropyl), preferably methyl.

In accordance with other embodiments of the invention, the compounds are provided in accordance with the following Formula IId or pharmaceutically acceptable salts thereof:

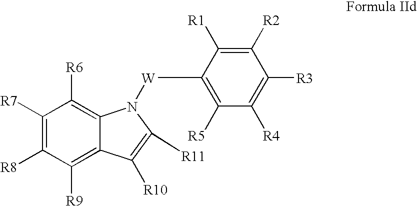

wherein W is —CH₂— or —CH(C₁₋₆ alkyl)-, and preferably —CH₂—;
R1, R2, R4, R5, R6, R7, R9 and R10 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂; or C₁₋₆ alkyl-S— optionally substituted with 1, 2, 3, and 4-6 halo (e.g., F, Cl, Br, I), preferably —SCF₃;
R3 is selected from the group consisting of C₁₋₃ haloalkyl (e.g., —CHF₂, —CF₃), —SCF₃, C₁₋₃ alkoxy, or C₁₋₃ haloalkoxy (e.g., —OCF₃, —OCHF₂), wherein optionally R₃ forms a 5 or 6-membered heterocycle with the adjacent R2 or R4 group;
R8 is H; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably ethoxy, propyloxy and isopropyloxy; C₁₋₆ alkyl-S— optionally substituted with 1, 2, 3, and 4-6 halo (e.g., F, Cl, Br, I); or —S(O)₂—(C₁₋₆ alkyl); —NO₂;
R11 is selected from the group consisting of —R^{L}—C(═O)R₄₂, —R^{L}—C(═S)R₄₂, —R^{L}—C(═O)S—R₄₃, —R^{L}—C(═O)N(R₅₂)(R₅₃), —S(O)₂—(C₁₋₆ alkyl); —R^{L}-phosphono, and —R^{L}-tetrazolyl;
R^{L} is selected from a bond, C₁₋₆ alkyl, C₂₋₆ alkenyl and C₂₋₆ alkynyl, preferably a bond or C₁ alkyl;
R₄₂ is selected from H, —OH, C₁₋₆ alkyl, C₂₋₆ alkenyl, C₂₋₆ alkynyl, C₁₋₆ alkoxy, C₂₋₆ alkenyloxy, C₂₋₆ alkynyloxy, and C₁₋₆ alkylthiol, wherein R₄₂ is optionally substituted with from one to three substituents independently selected from halo, N₃, nitro, hydroxy, thiol, CN and C₁₋₆ alkyl;
R₄₃ is H, C₁₋₆ alkyl, C₂₋₆ alkenyl or C₂₋₆ alkynyl, wherein R₄₃ is optionally substituted with from one to three substituents independently selected from halo, N₃, nitro, hydroxy, thiol, CN and C₁₋₆ alkyl; and
R₅₂ and R₅₃ are independently H, OH(R₅₂ and R₅₃ are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, or R₅₂ and R₅₃ together with the nitrogen atom to which they are both linked form a 3, 4, 5 or 6-membered heterocycle (e.g., piperidinyl, pyrrolidinyl, and morpholinyl), wherein R₅₂ and R₅₃ each is optionally substituted with 1-3 substituents wherein each substituent is independently halo, N₃, nitro, hydroxy, thiol, CN, C₁₋₆ alkyl, C₁₋₆ haloalkyl, C₁₋₆ alkoxy, —C(═O)N(R₅₄)(R₅₅), R₄₄C(═O)— or —N(R₅₄)(R₅₅), wherein R₅₄ and R₅₅ are independently H, OH or C₁₋₄ alkyl, and wherein R₄₄ is H or C₁₋₄ alkyl.
   Examples of R₁₁ moiety include —C(═O)OH, —CH═CHC(═O)OH, —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —C(═O)NHOH, —C(═O)CH₂NH₂, —C(═O)CH₂OH, —C(═O)CH₂SH, —C(═O)NHCN, -sulfone, -(2,6 difluorophenol), -phosphono, and -tetrazolyl.

In a preferred embodiment of the compounds of Formula IId,
W is —CH₂—;
R1, R2, R4, R5, R6, R7, R9 and R10 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂; or C₁₋₆ alkyl-S— optionally substituted with 1, 2, 3, and 4-6 halo (e.g., F, Cl, Br, I), preferably —SCF₃;
R3 is selected from the group consisting of —CHF₂, —CF₃, —OCF₃, or —OCHF₂;
R8 is H; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably ethoxy, propyloxy and isopropyloxy; C₁₋₆ alkyl-S— optionally substituted with 1, 2, 3, and 4-6 halo (e.g., F, Cl, Br, I); or —S(O)₂—(C₁₋₆ alkyl); —NO₂;
R11 is selected from the group consisting of —R^{L}—COOH; and
R^{L} is selected from a bond, C₁₋₆ alkyl, C₂₋₆ alkenyl and C₂₋₆ alkynyl, preferably a bond (i.e., R11 is —COOH).

In preferred embodiments, R1, R2, R4, R5, R6, R7, R9 and R10 are each H or halo, preferably H.

In preferred embodiments, R3 is selected from the group consisting of C₁₋₃ alkoxy, C₁₋₃ haloalkyl and C₁₋₃ haloalkoxy. In more preferred embodiments, R3 is —CF₃ or —OCF₃.

In preferred embodiments, R8 is selected from the group consisting of H, halo, C₁₋₃ alkoxy, C₁₋₄ alkyl, and C₁₋₃ haloalkoxy. In more preferred embodiments, R8 is F, Cl, —OCF₃, —CH₃, —OCH₃.

Compounds of the invention include those of Formula III below:

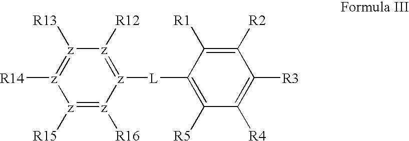

wherein one or more of R1-R5 is selected from the group consisting of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, L-C(═O)CH₂SH, -L-C(═O)NHCN, -L-sulfo, -L-(2,6 difluorophenol), -L-phosphono, and -L-tetrazolyl, and the others of R1-R5, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R12-R16, independent of one another, are selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

L can be saturated, partially saturated, or unsaturated, and is selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙO(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl; and

Each Z is independently selected from the group consisting of a carbon atom, and a nitrogen atom.

A preferred subset of compounds of Formula III for use in the invention include those where L represents a bond; Z1-Z6 are each C; R1 is hydro; R2 is selected from the group consisting of hydro, —C(═O)OH, —CH(CH₃)C(═O)OH; R3 is selected from the group consisting of hydro, —CH₂C(═O)OH, —CH(CH₃)C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, and —C(CH₂CH₃)₂C(═O)OH; R4 is selected from the group consisting of hydro, —CH₂C(═O)OH, —CH(CH₃)C(═O)OH, —C(CH₃)₂C(═O)OH, and —C(CH₂CH₃)₂C(═O)OH; R5 is selected from the group consisting of hydro or halo (if halo then preferably fluoro); R12 and R16 are each independently selected from the group consisting of halo and hydro; R13 and R15 are each independently selected from hydro and halo (if halo then preferably chloro); R14 is selected from the group consisting of hydro, halo, methoxy, and lower alkoxy; with the provision that the compound is not flurbiprofen, R-flurbiprofen, or S-flurbiprofen.

Another preferred subset of compounds of Formula III for use in the invention include those where L represents a bond; Z1-Z6 are each a carbon; R5 is selected from the group consisting of hydro or halo (if halo then preferably fluoro); R1 and R2 are each hydro; R3 is selected from the group consisting of hydro, —C(CH₃)₂C(═O)OH, and —C(CH₂CH₃)₂C(═O)OH; R4 is selected from the group consisting of hydro, —CH₂C(═O)OH, —CH(CH₃)C(═O)OH, —C(CH₃)₂C(═O)OH, and —C(CH₂CH₃)₂C(═O)OH; R12 and R16 are each hydro; R13 and R15 are selected from hydro and halo (if halo then preferably chloro); R14 is selected from the group consisting of hydro, methoxy, and lower alkoxy.

In another preferred subset of compounds of Formula III, L is selected from the group consisting of —O— and —NH—; R1 is selected from the group consisting of hydro, —CH₂C(═O)OH, —CH(CH₃)C(═O)OH, —C(CH₃)₂C(═O)OH, —C(CH₂CH₃)₂C(═O)OH, and —C(═O)OH;

R2 is selected from the group consisting of hydro, —CH₂C(═O)OH, —CH(CH₃)C(═O)OH, —C(CH₃)₂C(═O)OH, —C(CH₂CH₃)₂C(═O)OH, and —C(═O)OH; R3 is hydro; R4 is hydro; R5 is hydro; R12 is selected from hydro or halo (if halo then preferably chloro); R13 is selected from hydro, halo (if halo then preferably chloro), —CF₃, and —CH₃; R14 is hydro; R15 is hydro or halo (if halo then preferably chloro); and R16 is hydro or halo (if halo then preferably chloro).

In yet another preferred subset of compounds of Formula III for use in the invention, L is —NH—CH₂—; R1 is hydro; R2 is selected from halo, —CH₃, and —CF₃; R3 is hydro or halo (if halo then preferably chloro); R4 is selected from halo, —CH₃, and —CF₃; R5 is hydro; R12 is —C(═O)OH; R13 is hydro, R14 is —NO₂; R15 is hydro; and R16 is hydro.

In still another preferred subset of compounds of Formula III for use in the invention, L is selected from —NH—CH₂—, —NH—CH₂—CH₂—, —NH—CH₂—CH₂—CH₂—, —NH—CH(CH₃)—, —NH—CH(CH₃)—CH₂—, —NH—CH(CH₃)—CH₂—CH₂—, —NH—CH₂—CH(CH₃)—, and —NH—CH₂—CH₂—CH(CH₃)—; R1 is selected from the group consisting of hydro and halo (if halo then preferably chloro); R2 is selected from the group consisting of hydro, halo, haloalkyl (preferably trifluoromethyl), alkoxy (preferably methoxy), alkyl (preferably methyl); R3 is selected from the group consisting of hydro, halo, and phenyl; R4 is selected from the group consisting of hydro, halo, haloalkyl (preferably trifluoromethyl), alkoxy (preferably methoxy), alkyl (preferably methyl); R5 is selected from the group consisting of hydro and halo (if halo then preferably chloro); R12 is —C(═O)OH; R13 is hydro; R14 is —NO₂; R15 is hydro; and R16 is hydro. Additionally, in this subset of compounds any two of R1-R5 can be taken together to form an optionally substituted aryl or heteroaryl ring.

Another preferred subset of compounds of Formula III for use in the invention include those where L is a bond, each of R1-R5 is independently selected from the group consisting of hydro or —CH₂—C(═O)OH; each of Z1-Z6 is independently selected from the group consisting of C or N; R12 is selected from the group consisting of lower alkoxy, methoxy, ethoxy, halo, fluoro, and chloro; R13 is selected from the group consisting of lower alkoxy, methoxy, ethoxy, halo, fluoro, and chloro; R14 is selected from the group consisting of lower alkoxy, methoxy, ethoxy, halo, fluoro, and chloro; R15 is selected from the group consisting of lower alkoxy, methoxy, ethoxy, halo, fluoro, and chloro; R16 is selected from the group consisting of lower alkoxy, methoxy, ethoxy, halo, fluoro, and chloro.

In a preferred embodiment, the treatment methods of the present invention comprises administering a compound according to structure (IIa)

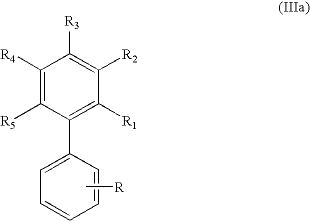

or pharmaceutically acceptable salt thereof, wherein

R and R₁-R₅ are selected from the group consisting of H, OH, halo, alkyl, and alkoxy provided that one of R₂-R₄ is C(Rₓ)(R_{y})COOH wherein Rₓ and R_{y} are independently H alkyl, or alkenyl.

In one embodiment of structure (IIIa), Rₓ and R_{y} are both H. In one embodiment of structure (III), R₃ is H. In a specific embodiment of structure (III), R₂ and R₄ are H. In specific embodiments of structure (III), R₁ and R₅ are H or F, and R is H, F, or alkoxy.

Compounds of the invention include those of Formula IV below:

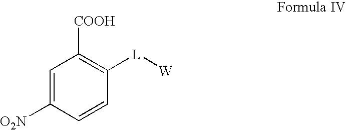

L can be saturated, partially saturated, or unsaturated, and is selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙO(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl; and

W is selected from the group consisting of optionally substituted cycloalkyl, optionally substituted aryl, optionally substituted heterocycle, and optionally substituted heteroaryl.

Optionally substituted, when used herein without reference to further definition, refers to a substituent selected from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In one embodiment, the compounds of Formula IV is according to structure IVa

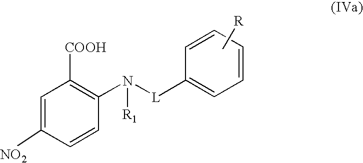

or pharmaceutically acceptable salt thereof are used in the treatment methods of the present invention, wherein

L is (CH₂)₁₋₄ optionally substituted by one or more C₁₋₆ alkyl moieties;

R represents one or moieties selected from the group consisting of halo, alkyl, haloalkyl, alkoxy, NO₂, amino optionally substituted by one or more alkyl moieties, and phenyl; and

R₁ is H or alkyl.

Preferably, R represents one or moieties selected from the group consisting of alkyl and haloalkyl, with the proviso that the compound is not 2-(3-trifluoromethylbenzyl amino)-5-nitrobenzoic acid.

In preferred embodiments, the compounds used in the treatment methods of present invention has the structure (IVb)

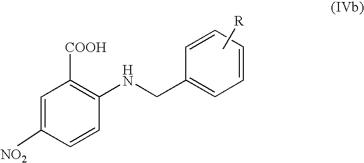

or pharmaceutically acceptable salt thereof, wherein

R represents one or moieties selected from the group consisting of halo, alkyl, and haloalkyl.

In more preferred embodiments, compounds are provided according to structure (IVb) wherein R represents one or moieties selected from the group consisting of alkyl and haloalkyl, with the proviso that the compound is not 2-(3-trifluoromethylbenzyl amino)-5-nitrobenzoic acid.

In embodiments of structures IVa and IVb, R is attached at the meta or para position. In specific embodiments of structures IVa and IVb, R is chloro, methyl, bromo, or trifluoromethyl.

Also provided are compounds having according to structure (V) useful in the methods of the present invention:

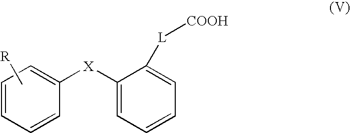

or a pharmaceutically acceptable salt thereof, wherein

L is (CH₂)₀₋₁ optionally substituted by C₁₋₃ alkyl;

X is O, OCH₂, or NR₁ wherein R₁ is H or C₁₋₃ alkyl; and

R represents one or moieties selected from the group consisting of H, halo, C₁₋₃ alkyl, C₁₋₃ alkoxy, NH₂, COOH, and phenyl.

In one embodiment of structure (V), the compound has a structure according to structure (Va)

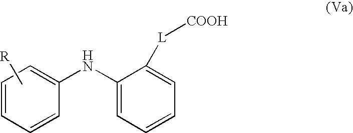

wherein L and R are as defined for structure (V).

In a specific embodiment of structures V and Va, L is CH₂. In specific embodiments of structures V and Va, R is halo.

### Methods of Treatment and Prevention

The invention provides methods for treating and/or preventing neurodegenerative disorders like AD and MCI, and lowering Aβ42 in an individual in need of such treatment. It is believed that by lowering the amounts of Aβ42 in an individual by administering an Aβ342 lowering effective amount of a composition described herein, that Alzheimer's disease and mild cognitive impairment can be treated or prevented. Generally, the invention relates to the idea that compounds of Formula I-Va can be used to lower Aβ42 levels. Thus, diseases characterized by increased levels of Aβ42, can be treated or prevented with the methods of the invention which are designed to lower Aβ42, prevent an increase in Aβ42, and/or reduce the rate of increase of Aβ42.

The invention is based on the fact that the inventors have discovered that compounds of the formulae above lower Aβ42 Levels in in vitro APP processing assays. Furthermore, the compounds, in general, have negligible levels of COX inhibition and therefore are thought to essentially be devoid of the deleterious side-effects associated with COX inhibition. Thus, a preferred embodiment of the invention is the use of a pharmaceutical composition having one or more compounds of the above formulae, where the compound lowers Aβ42 levels and does not substantial inhibit the cyclooxygenases. Preferred compounds of the formulae for use in the invention are those that have little or negligible COX1 and/or COX2 inhibition at 1 μM, more preferred are those that little or negligible COX1 and/or COX2 inhibition at 10 μM, and more preferred are those that little or negligible COX1 and/or COX2 inhibition at 100 μM compound. COX1 and COX2 inhibition can be determined with a COX inhibitor screening kit from e.g., Cayman Chemical, Ann Arbor, Mich. (Cat. #560131).

In one embodiment of the invention, a method for lowering Aβ₄₂ protein levels, in an individual in need of such treatment, is provided that includes the step of administering an effective amount of a compound of one of the above formulae, as described above.

While not wishing to be bound by theory, it is believed that the compounds of the above formulae act in vivo to lower cellular Aβ₄₂ production and/or secretion, and therefore can reduce Aβ₄₂ level (brain, CSF and/or plasma level) in mammals. Thus, it is useful in treating diseases and disorders amenable to reduction of cellular Aβ₄₂ production or secretion, such as neurodegenerative disorders (dementia, Alzheimer's disease, MCI, Parkinson's disease, Down's syndrome, etc.), inclusion body myositis, and tauopathies (corticobasal degeneration, and progressive supranuclear palsy). See Oddo et al., Neuron, 43:321-332 (2004). They are particularly useful in treating and/or preventing Alzheimer's disease and MCI by lowering the amount of Aβ₄₂ that is present or would be present in the absence of such treatment. Amyloid β polypeptides are derived from amyloid precursor proteins (APPs). A variety of amyloid β polypeptides are known including Aβ₃₄, Aβ₃₇, Aβ₃₈, Aβ₃₉, and Aβ₄₀. Increased Aβ₄₂ levels are associated with Alzheimer's disease and MCI. Thus, by lowering the amounts of Aβ₄₂, a treatment is provided for combating Alzheimer's disease and/or MCI.

In another embodiment, the invention relates to a method of delaying the onset of Alzheimer's disease or MCI, or one or more symptoms thereof, or slowing the progress of Alzheimer's disease or MCI, which comprises administering, to an individual in need of such treatment, a composition comprising a compound having one of the above formulae.

In another embodiment, the invention provides a method of treating a neurodegenerative disorder, by identifying a patient in need of such treatment, and administering to the patient a therapeutically effective amount of a pharmaceutical composition having one or more compounds of one of the above formulae. Administration of a compound of one of the above formulae for at least 4 weeks, preferably at least 4 months, and more desirably at least 8 months, can provide an improvement or lessening in decline of cognitive function as characterized by cognition tests, biochemical disease marker progression, and/or plaque pathology. It is preferred that the lessening in decline in cognitive function is at least 25% as compared to individuals treated with placebo, more preferably at least 40%, and even more desirably at least 60%. For example, an individual treated with placebo having probable mild-to-moderate Alzheimer's disease is expected to score approximately 5.5 points lower on the ADAS-cog test after a specified period of time of treatment (e.g., 1 year) whereas an individual treated with the composition of this aspect of the invention for the same period of time will score approximately 2.2 points lower on the ADAS-cog scale with a 60% decrease in decline or 3.3 points lower with a 40% decrease in decline in cognitive function when treated with the composition for the same specified period of time. The pharmaceutical composition for use in the invention is formulated with one or more pharmaceutically acceptable excipients, salts, or carriers. The pharmaceutical composition for use in the invention is delivered orally, preferably in a tablet or capsule dosage form.

In yet another embodiment, the invention provides a method for prophylaxis against a neurodegenerative disorder, by identifying a patient in need of or desiring such treatment, and administering to the patient a prophylactically effective amount of a pharmaceutical composition having one or more compounds of one of the above formulae. Preferred compounds for use in this embodiment of the invention include those in Tables 1-7. Administration of the compound for at least 4 weeks, preferably at least 4 months, and more desirably at least 8 months, can delay the onset of the neurodegenerative disorder or slow the rate of onset of symptoms of the disorder. Patients having a predisposition to a neurodegenerative disorder or suspected of needing prophylaxis can be identified by any method known to the skilled artisan for diagnosis of such neurodegenerative disorders.

In still another embodiment, the invention provides a method of treating a disease characterized by abnormal amyloid precursor protein processing by (1) identifying a patient in need of such treatment, and (2) administering to the patient a therapeutically effective amount of a pharmaceutical composition having one or more compounds of one of the above formulae. Examples of biochemical disease markers include, for example, amyloid beta peptide (Aβ), Aβ₄₂, and tau.

In another embodiment, the invention provides a method of prophylaxis or delaying the onset of a disease (or one or more symptoms thereof) characterized by abnormal amyloid precursor protein processing, by identifying a patient in need of such treatment and administering to the patient a prophylactically effective amount of a pharmaceutical composition having one or more compounds of one of the above formulae. Oral administration of the pharmaceutical composition for use in the method of this aspect the invention for at least 4 weeks, preferably at least 4 months, and more desirably at least 8 months, prevents or delays the onset of the disease (or symptoms thereof) characterized by abnormal amyloid precursor protein processing.

In another embodiment, the invention provides a method of treating Alzheimer's disease comprising administering to a patient in need of such treatment, a pharmaceutical composition having one or more compounds of one of the above formulae. Oral administration of the pharmaceutical composition for use in the method of this aspect of the invention for at least 4 weeks, preferably at least 4 months, and more desirably at least 8 months, provides an improvement or lessening in decline of cognitive function as characterized by cognition tests, biochemical disease marker progression, and/or plaque pathology. Desirably, the oral dose is provided in capsule or tablet form. According to this aspect of the invention, a patient in need of treatment is administered an Alzheimer's disease treating effective amount of a pharmaceutical composition having one or more compounds of one of the above formulae and one or more pharmaceutically acceptable salts, excipients and carriers. The method of this aspect of the invention involves identifying an individual likely to have mild-to-moderate Alzheimer's disease. An individual having probable mild-to-moderate Alzheimer's disease can be diagnosed by any method available to the ordinary artisan skilled in such diagnoses. For example, diagnosis can be according to DSM IV (TR) and/or meets NINCDS-ADRDA criteria for probable AD. According to this aspect of the invention, individuals with probable mild-to-moderate AD take an oral dose of a pharmaceutical composition for a specified period of time. Individuals undergoing such treatment are likely to see an improvement or lessening in decline of cognitive function, an improvement or lessening in decline in biochemical disease marker progression, and/or an improvement or lessening of decline in plaque pathology. A lessening in decline in cognitive function can be assessed using tests of cognitive function like the ADAS-cog. For example, an individual treated with placebo having probable mild-to-moderate Alzheimer's disease is expected to score approximately 5.5 points lower on the ADAS-cog test after a specified period of time of treatment (e.g., 1 year) whereas an individual treated with the composition of this aspect of the invention for the same period of time will score approximately 2.2 points lower on the ADAS-cog scale with a 60% decrease in decline or 3.3 points lower with a 40% decrease in decline in cognitive function when treated with the composition for the same specified period of time. In a related aspect, the method involves identifying a patient having moderate-to-severe AD and administering to the patient an Alzheimer's disease treating effective amount of a compound of one of the above formulae.

In another embodiment, the invention provides a method of preventing the onset of Alzheimer's disease comprising administering to a patient in need of or desiring such treatment, a pharmaceutical composition having one or more compounds of one of the above formulae. Administration of the pharmaceutical composition for use in the method of this aspect of the invention for at least 4 weeks, preferably at least 4 months, and more desirably at least 8 months, delays the onset of decline of cognitive function, biochemical disease marker progression, and/or plaque pathology. According to this embodiment, an individual desiring or needing preventative treatment against the onset of AD is administered a pharmaceutical composition having one or more compounds of one of the above formulae. The preventative treatment is preferably maintained as long as the individual continues to desire or need the treatment. Individuals needing or desiring preventative treatment against AD can be those having risk factors for developing AD. For example, risk factors for developing AD can be genetic factors or environmental factors. In one embodiment, the risk factor is age. Genetic risk factors can be assessed in a variety of ways, such as ascertaining the family medical history of the individual, or performing a genetic test to identify genes that confer a predisposition for developing AD. Additionally, risk factors can be assessed by monitoring genetic and biochemical markers. The method of this embodiment involves evaluating risk factors for cognitive decline. Evaluation of risk factors can include genetic testing for predisposing genes, alleles, and polymorphisms. Risk factors also refer to environmental factors like stroke, brain injury, age, and diet. Depending on the risk factor or factors associated with a particular patient a particular treatment regimen is selected for treating cognitive decline. For example, mutations in a Familial Alzheimer's disease gene are a risk factor. Another risk factor for cognitive decline is age. Head trauma is another risk factor for cognitive decline. Based on the patient's risk factors, a physician will prescribe a particular therapeutic treatment or prophylactic treatment suitable for the patient.

In still another embodiment, the invention provides a method of lowering Aβ42 levels to a greater extent than inhibiting COX-1, COX-2, or a combination thereof. In particular, the method of this embodiment comprises administering to a patient in need of treatment an effective amount of one or more compounds of one of the above formulae. The method of this embodiment involves the lowering of Aβ42 levels while not substantial affecting the activity of COX-1, COX-2, or both COX-1, and COX-2. Thus, the amount of the composition administered is effective for lowering Aβ₄₂ levels and does not substantially inhibit COX-1, COX-2, or both COX-1 and COX-2. For example, the effective amount can be above the ED50 (the dose therapeutically effective in 50% of the population) for Aβ₄₂ lowering, and below the ED50 for COX inhibition. Another example is a sufficiently small amount of compound so that inhibition of at least one COX activity is negligible and Aβ₄₂ levels are reduced. The method of this embodiment can be used to treat and/or prevent Alzheimer's disease. The method of this embodiment can also be used to treat and/or prevent MCI and other neurodegenerative disorders.

According to a preferred embodiment, the invention provides a method of lowering Aβ₄₂ levels to a greater extent than inhibiting COX-1, COX-2, or a combination thereof. In particular, the method of this embodiment comprises administering, to a patient in need of treatment, an effective amount of one or more compounds of one of the above formulae, wherein the effective amount of compound is capable of lowering Aβ₄₂, while not substantially affecting or inhibiting the activity of at least one isoform of COX. Thus, the method of this embodiment involves the lowering of Aβ₄₂ levels while not substantially inhibiting the activity of COX-1, COX-2, or both COX-1 and COX-2. The method of this embodiment can be used to treat and/or prevent Alzheimer's disease, MCI, and/or other neurodegenerative disorders. In one aspect of this embodiment, the effective amount of a compound of one of the above formulae reduces Aβ₄₂ levels or production of Aβ₄₂ by at least 1, 2, 5, 10, 15, 20, 25, 30, 40, or 50 or more percent while inhibiting COX-1, COX-2, or both COX-1 and COX-2 by less than 1, 2, 5, 10, 15, 20, 25, 30, 40, 50, 60, 70, 80, or 90 percent. In a preferred aspect of this embodiment, the effective amount of compound according to one of the above formulae lower Aβ₄₂ by at least 5 percent while not substantially inhibiting COX-1, COX-2, or both COX-1 and COX-2 activity or levels. In another preferred aspect of this embodiment, the effective amount of a compound of one of the above formulae that is administered to an individual is such that it lowers Aβ₄₂ levels, and does not inhibit COX activity to a significant extent, e.g., the amount administered is below the in vivo IC50 value for COX-1, COX-2 or both COX-1 and COX-2 and above the in vivo IC50 value for Aβ₄₂ lowering activity. As used in this context, IC50 refers to the amount of compound sufficient to inhibit COX activity by 50% (COX-1, COX-2, or both COX-1 and COX-2) or reduce Aβ₄₂ levels by 50%. An “effective amount” according to this preferred aspect of this embodiment, can also be viewed in terms of ED50 parameters, binding constants, dissociation constants, and other pharmacological parameters, e.g., the amount administered is below the ED50 value for COX-1, COX-2 or both COX-1 and COX-2 and above the ED50 value for Aβ₄₂. It is noted that the effective amount of the compound does not necessarily have to be above an IC50 or ED50 for Aβ₄₂ lowering and below the IC50 or ED50 for COX inhibition. That is, the “effective amount” can be at some intermediate value such that Aβ₄₂ levels are lowered to a greater extent than inhibition of COX-1, COX-2 or both COX-1 and COX-2.

### Patient Population

Any individual having, or suspected of having, a neurodegenerative disorder, such as Alzheimer's disease, may be treated using the compositions and methods of the present invention. Individuals who would particularly benefit from the compositions and methods of the invention include those individuals diagnosed as having mild to moderate Alzheimer's disease according to a medically-accepted diagnosis, such as, for example the NINCDS-ADRDA criteria. Progression of the disease may be followed by medically accepted measure of cognitive function, such as, for example, the Mini-Mental State Exam (MMSE; see Mohs et al. Int. Psychogeriatr. 8:195-203 (1996)); ADAS-Cog (Alzheimer Disease Assessment Scale-Cognitive; see Galasko et al. Alzheimer Dis Assoc Disord, 11 suppl 2:S33-9 (1997)); Behavioral Pathology in Alzheimer's Disease Rating Scale (BEHAVE-AD); Blessed Test; CANTAB—Cambridge Neuropsychological Test Automated Battery; CERAD (The Consortium to Establish a Registry for Alzheimer's Disease) Clinical and Neuropsychological Tests (includes MMSE); Clock Draw Test; Cornell Scale for Depression in Dementia (CSDD); Geriatric Depression Scale (GDS); Neuropsychiatric Inventory (NPI); the 7 Minute Screen; the Alzheimer's Disease Cooperative Study Activities of Daily Living scale (ADCS-ADL; see McKhann et al. Neurology 34:939-944 (1984)); the DSM-IV (Diagnostic and Statistical Manual of Mental Disorders—Fourth Edition (DSM-IV), published by the American Psychiatric Association, Washington D.C., 1994); or the NINCDS-ADRDA criteria (see Folstein et al. J. Psychiatr. Res. 12:189-198 (1975)). Individuals diagnosed as having probable AD can be identified as having a mild-to-moderate form of the disease by an accepted measure of cognitive function such as the MMSE. In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993). The severity of AD is generally determined by one of the initial tests provided above. For example, MMSE scores of 26-19 indicate mild AD, while scores from 18-10 indicate moderate AD.

Diagnoses of Alzheimer's disease based on these tests are recorded as presumptive or probable, and may optionally be supported by one or more additional criteria. For example, a diagnosis of Alzheimer's disease may be supported by evidence of a family history of AD; non-specific changes in EEG, such as increased slow-wave activity; evidence of cerebral atrophy on CT with progression documented by serial observation; associated symptoms such as depression, insomnia, incontinence, delusions, illusions, hallucinations, catastrophic verbal, emotional or physical outbursts, sexual disorders, weight loss, and/or attendant neurologic abnormalities, such as increased muscle tone, myoclonus or gait disorder, etc.

Additionally, amyloid deposits, generally associated with AD, may be detected through the use of positron emission tomography (PET) using an amyloid-specific tracer such as Pittsburgh Compound-B (PIB). See Klunk et al., Ann. Neurol. 55(3):306-309 (2004). Increased amyloid deposits in the frontal, parietal, temporal and occipital cortices, and in the striatum, relative to normal brain tissue, as visualized, for example by PIB, support a diagnosis of AD. Generally, a greater number and density of amyloid deposits indicates more advanced AD.

The invention encompasses the treatment of an individual preferably having mild to moderate AD, to the extent that individual has AD, whether or not one or more non-AD neurodegenerative diseases or conditions are previously, concurrently or subsequently diagnosed.

The compounds and methods of the present invention are useful for individuals who have received prior medication for AD, as well as individuals who have received no prior medication for AD, and is useful for individuals currently receiving medication for AD other than a compound of Formula I-Va, and for individuals not receiving medication for AD other than a compound of Formula I-Va.

Individuals of any age may be treated by the methods of the invention, with the pharmaceutical compositions of the invention; however, the invention encompasses a preferred embodiment for treating or preventing Alzheimer's disease in individuals between the ages of 55 and 80. In various embodiments, individuals treated by the therapeutic or prophylactic methods of the invention may be from 55 to 70 years of age, 60 to 80 years of age, 55 to 65 years of age, 60 to 75 years of age, 65 to 80 years of age, 55 to 60 years of age, 60 to 65 years of age, 65 to 70 years of age, 70 to 75 years of age, 75 to 80 years of age, or 80 years old and older.

In yet another embodiment, the invention provides a method of slowing cognitive decline in an individual suspected of having mild cognitive impairment (MCI) comprising administering to the individual an effective amount of a compound of Formula I-Va. Mild cognitive impairment is a clinical condition between normal aging and Alzheimer's disease characterized by memory loss greater than expected for the particular age of the individual yet the individual does not meet the currently accepted definition for probable Alzheimer's disease. See, e.g., Petersen, et al. Arch. Neurol. 58:1985-1992 (2001); Petersen, Nature Rev. 2:646-653 (2003); and Morris et al. J. Mol. Neuro. 17:101-118 (2001). Thus, according to this embodiment an individual suspected of having or diagnosed with MCI is treated twice daily with a composition having a compound of Formula I-Va per dose for at least 4 weeks, at least 4 months, preferably at least 8 months, and more desirably at least 1 year. Typically, patients having MCI first complain of or have a loss of memory. Preferably an individual associated with the patient can corroborate the memory deficit. Furthermore, general cognition is not sufficiently impaired to cause concern about more widespread cognitive disorder and although daily living activities may be affected that are not significantly impaired and the patients are not demented. Individuals having or suspected of having MCI that are treated according to this embodiment can expect to slow cognitive decline and/or progression to probable AD.

Thus, in one embodiment, the invention provides a method of treating an individual known or suspected of having Alzheimer's disease comprising administering an effective amount of a compound of Formula I-Va. In a specific embodiment, said individual is diagnosed as having mild to moderate Alzheimer's disease. In a more specific embodiment, said individual is diagnosed by a cognitive test as having mild to moderate AD. In a more specific embodiment, said cognitive test is the Mini-Mental State Exam (MMSE). In an even more specific embodiment, said individual has a score in said MMSE of from 26 to 19, inclusive. In another more specific embodiment, said individual has a score in said MMSE of from 18 to 10, inclusive. In another specific embodiment, said individual has a score in said MMSE of 26 to 10, inclusive.

In other embodiments, the invention provides a method of treating an individual known or suspected of having Alzheimer's disease comprising administering an effective amount of a compound of Formula I-Va, wherein said individual is concurrently taking a second drug for the treatment of Alzheimer's disease. In a further embodiment, said individual has been diagnosed as having mild to moderate Alzheimer's disease. In a specific embodiment, said second drug is an acetylcholinesterase (AChE) inhibitor. In a more specific embodiment, said AChE inhibitor is Galanthamine (galantamine, Reminyl); E2020 (Donepezil, Aricept); Physostigmine; Tacrine (tetrahydroaminoacridine, THA); Rivastigmine; Phenserine; Metrifonate (Promem); or Huperazine, or a combination of any of the foregoing. In another embodiment, said second drug is a drug other than an acetylcholinesterase inhibitor. In a preferred embodiment, the method or compositions of the invention are used in patients or individuals undergoing therapy with Aricept. The invention also encompasses methods of treating patients refractory to, or who no longer show improvement with, conventional AD therapy.

In another embodiment, said individual is concurrently taking a non-drug substance for the treatment of Alzheimer's disease. In a specific embodiment, said non-drug substance is an anti-oxidant. In a more specific example, said anti-oxidant is vitamin C or vitamin E. In an even more specific embodiment, said vitamin C is taken in a dose of 500-1000 mg per dose of a compound of Formula I-Va. In another even more specific embodiment, said vitamin E is taken in a dose of 400-800 IU per dose of a compound of Formula I-Va. In this regard, the invention encompasses the use of one or more such anti-oxidants as an adjunct to therapy for Alzheimer's disease, and not primarily as a nutritional supplement.

In another embodiment, the invention provides a method of treating an individual diagnosed as having mild to moderate Alzheimer's disease comprising administering an effective amount of a compound of Formula I-Va, wherein said individual has, prior to taking a compound of Formula I-Va, taken a second drug for the treatment of Alzheimer's disease. In a specific embodiment, said second drug is an acetylcholinesterase (AChE) inhibitor. In a more specific embodiment, said ACE inhibitor is Galanthamine (galantamine, Reminyl); E2020 (Donepezil, Aricept); Physostigmine; Tacrine (tetrahydroaminoacridine, THA); Rivastigmine; Phenserine; Metrifonate (Promem); or Huperazine, or a combination of any of the foregoing. In another embodiment, said second drug is a drug other than an acetylcholinesterase inhibitor.

In another embodiment, said individual has, prior to taking a compound of Formula I-Va, taken a non-drug substance for the treatment of Alzheimer's disease. In a specific embodiment, said non-drug substance is an anti-oxidant. In a more specific example, said anti-oxidant is vitamin C or vitamin E. In an even more specific embodiment, said vitamin C is taken in a dose of 500-1000 mg per dose. In another even more specific embodiment, said vitamin E is taken in a dose of 400-800 IU per dose. In this regard, the invention encompasses the use of one or more such anti-oxidants as an adjunct to therapy for Alzheimer's disease, and not primarily as a nutritional supplement.

The invention further provides a combination therapy strategy for preventing Alzheimer's disease and MCI. According to this aspect of the invention, an individual in need of treatment is administered a compound of Formula I-Va, and a compound selected from the group consisting of NSAIDs (non-steroidal anti-inflammatory drugs), COX-2 inhibitors (cyclooxygenase-2), β-secretase inhibitors, R-flurbiprofen, γ-secretase inhibitors, acetylcholine esterase inhibitors, and NMDA antagonists. Preferably the combination therapy involves treating the individual in need of treatment with a compound of Formula I-Va in combination with an acetylcholine esterase inhibitor or an NMDA receptor antagonist. Preferred acetylcholine esterase inhibitors for combination therapy are tacrine, donepezil, rivastigmine, and galantamine. Preferred NMDA receptor antagonists for combination therapy are memantine, adamantane, amantadine, an adamantane derivative, dextromethorphan, dextrorphan, dizocilpine, ibogaine, ketamine, and remacemide. The acetylcholine esterase inhibitor or NMDA receptor antagonists is preferably formulated in a combination dosage form with a compound of Formula I-Va.

The treatment regime used in the combination therapy can involve administration of a composition comprising the combination of active ingredients, the concomitant administration of separate compositions, each comprising at least one active ingredient. Furthermore, the administration of the active ingredients can be performed at different times and/or different routes. For example, a composition comprising at least one active ingredient can be administered in the morning, and a composition comprising at least one different active ingredient can be administered in the evening. Another example would involve the administration of a composition having at least one active ingredient orally while the second composition is administered intravenously.

While not wishing to be bound by theory, it is believed that the compounds of Formula I-Va are capable of slowing the rate of death of neurons. Accordingly, it is also believed that the compounds of Formula I-Va acts in vivo to treat and/or prevent Alzheimer's disease and MCI by slowing the rate of death of neurons that is present or would be present in the absence of such treatment.

The skilled artisan readily recognizes that the invention includes the use of compounds of Formula I-Va, pharmaceutically acceptable salts, metabolites and prodrugs thereof in each of the described embodiments.

### DEFINITIONS

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

As used herein, the term “alkyl” refers to a saturated aliphatic hydrocarbon including straight chain and branched chain groups. Preferably, the alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). More preferably, it is a medium size alkyl having 1 to 10 carbon atoms. Even more preferably, it is a lower alkyl having 1 to 6 carbon atoms, and even more preferably 1 to 4 carbon atoms. The alkyl group may be substituted or unsubstituted. When substituted, the substituent group(s) is preferably one or more individually selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, cyanato, isocyanato, thiocyanato, isothiocyanato, nitro, silyl, and amino.

As used herein, the term “halo” refers to chloro, fluoro, bromo, and iodo.

As used herein, the term “hydro” refers to a hydrogen atom (—H group).

As used herein, the term “hydroxy” refers to an —OH group.

As used herein, the term “alkoxy” refers to both an —O-alkyl and an —O-cycloalkyl group, as defined herein. Lower alkoxy refers to —O-lower alkyl groups.

As used herein, the term “aryloxy” refers to both an —O-aryl and an —O-heteroaryl group, as defined herein.

As used herein, the term “mercapto” group refers to an —SH group.

As used herein, the term “alkylthio” group refers to both an S-alkyl and an —S-cycloalkyl group, as defined herein.

As used herein, the term “arylthio” group refers to both an —S-aryl and an —S-heteroaryl group, as defined herein.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)-group.

As used herein, the term “thiocarbonyl” group refers to a —C(═S)R″ group, with R″ as defined herein.

As used herein, the term “O-carboxy” group refers to a R″C(═O)O-group, with R″ as defined herein.

As used herein, the term “C-carboxy” group refers to a —C(═O)OR″ groups with R″ as defined herein.

As used herein, the term “ester” is a C-carboxy group, as defined herein, wherein R″ is any of the listed groups other than hydro.

As used herein, the term “C-carboxy salt” refers to a —C(═O)O⁻ M⁺ group wherein M⁺ is selected from the group consisting of lithium, sodium, magnesium, calcium, potassium, barium, iron, zinc and quaternary ammonium.

As used herein, the term “acetyl” group refers to a —C(═O)CH₃ group.

As used herein, the term “carboxyalkyl” refers to —(CH₂)ᵣC(═O)OR″ wherein r is 1-6 and R″ is as defined above.

As used herein, the term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

As used herein, the term “carboxylic acid” refers to a C-carboxy group in which R″ is hydro.

As used herein, the term “haloalkyl” refers to an alkyl group substituted with 1 to 6 halo groups; preferably haloalkyl is a —CX₃ group wherein X is a halo group. The halo groups can be independently selected.

As used herein, the term “trihalomethanesulfonyl” refers to a X₃ CS(═O)₂— group with X as defined above.

As used herein, the term “cyano” refers to a —C≡N group.

As used herein, the term “cyanato” refers to a —CNO group.

As used herein, the term “isocyanato” refers to a —NCO group.

As used herein, the term “thiocyanato” refers to a —CNS group.

As used herein, the term “isothiocyanato” refers to a —NCS group.

As used herein, the term “sulfinyl” refers to a —S(═O)R″ group, with R″ as defined herein.

As used herein, the term “sulfonyl” refers to a —S(═O)₂ R″ group, with R″ as defined herein.

As used herein, the term “sulfonamido” refers to a —S(═O)₂ NR¹⁷R¹⁸, with R¹⁷ and R¹⁸ as defined herein.

As used herein, the term “trihalomethanesulfonamido” refers to a X₃CS(═O)₂—NR¹⁷-group with X and R¹⁷ as defined herein.

As used herein, the term “O-carbamyl” refers to a —OC(═O)NR¹⁷R¹⁸ group with R¹⁷ and R¹⁸ as defined herein.

As used herein, the term “N-carbamyl” refers to a R¹⁸ OC(═O)NR¹⁷-group, with R¹⁷ and R¹⁸ as defined herein.

As used herein, the term “O-thiocarbamyl” refers to a —OC(═S)NR¹⁷ R¹⁸ group with R¹⁷ and R¹⁸ as defined herein.

As used herein, the term “N-thiocarbamyl” refers to a R¹⁷OC(═S)NR¹⁸-group, with R¹⁷ and R¹⁸ as defined herein.

As used herein, the term “amino” refers to an —NR¹⁷ R¹⁸ group, with R¹⁷ and R¹⁸ both being hydro.

As used herein, the term “C-amido” refers to a —C(═O)NR¹⁷ R¹⁸ group with R¹⁷ and R¹⁸ as defined herein. An “N-amido” refers to a R¹⁷ C(═O)NR¹⁸-group with R¹⁷ and R¹⁸ as defined herein.

As used herein, the term “nitro” refers to a —NO₂ group.

As used herein, the term “quaternary ammonium” refers to a —⁺NR¹⁷ R¹⁸ R¹⁹ group wherein R¹⁷, R¹⁸, and R¹⁹ are independently selected from the group consisting of hydro and unsubstituted lower alkyl.

As used herein, the term “methylenedioxy” refers to a —OCH₂O— group wherein the oxygen atoms are bonded to adjacent ring carbon atoms.

As used herein, the term “ethylenedioxy” refers to a —OCH₂CH₂O-group wherein the oxygen atoms are bonded to adjacent ring carbon atoms.

As used herein, the term “cycloalkyl” refers to an all-carbon monocyclic or fused ring (i.e., rings which share an adjacent pair of carbon atoms) group wherein one or more of the rings does not have a completely conjugated pi-electron system. Examples, without limitation, of cycloalkyl groups are cyclopropane, cyclobutane, cyclopentane, cyclopentene, cyclohexane, adamantane, cyclohexadiene, cycloheptane and, cycloheptatriene. A cycloalkyl group may be substituted or unsubstituted. When substituted, the substituent group(s) is preferably one or more individually selected from alkyl, aryl, heteroaryl, heterocyclic, hydroxy, alkoxy, aryloxy, mercapto, alkylthio, arylthio, cyano, halo, carbonyl, thiocarbonyl, carboxy, O-carbamyl, N-carbamyl, C-amido, N-amido, nitro, and amino.

As used herein, the term “heterocycle” refers to a mono or bicyclic ring that contains 4-12 atoms, at least one of which is selected from nitrogen, sulfur or oxygen, wherein a —CH₂— group can optionally be replaced by a —C(═O)—, and a ring sulfur atom may be optionally oxidized to form S-oxide(s). Suitably “heterocycle” is a monocyclic ring containing 5 or 6 atoms or a bicyclic ring containing 9 or 10 atoms. “Heterocycle” may be nitrogen or carbon linked. Example of “heterocycles” or “heterocyclic” rings include, but are not limited to, morpholino, piperidyl, piperazinyl, pyrrolidinyl, thiomorpholino, homopiperazinyl, imidazolyl, imidazolidinyl, pyrazolidinyl, dioxanyl and dioxolanyl. “Heterocycle” can include heteroaryls when the pi-electron system of a heterocycle is completely conjugated.

As used herein, the term “aryl” refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituted group(s) is preferably one or more selected from halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, S-sulfonamido, N-sulfonamido, trihalo-methanesulfonamido, and amino.

As used herein, the term “heteroaryl” refers to a monocyclic or fused ring (i.e., rings which share an adjacent pair of atoms) group having in the ring(s) one or more atoms selected from the group consisting of nitrogen, oxygen and sulfur and, in addition, having a completely conjugated pi-electron system. Examples, without limitation, of heteroaryl groups are pyrrole, furan, thiophene, imidazole, oxazole, thiazole, pyrazole, pyridine, pyrimidine, quinoline, isoquinoline, quinazoline, purine and carbazole. The heteroaryl group may be substituted or unsubstituted. When substituted, the substituted group(s) is preferably one or more selected from alkyl, cycloalkyl, halo, trihalomethyl, hydroxy, alkoxy, aryloxy, mercapto, alkylthio, arylthio, cyano, nitro, carbonyl, thiocarbonyl, sulfonamido, carboxy, sulfinyl, sulfonyl, O-carbamyl, N-carbamyl, O-thiocarbamyl, N-thiocarbamyl, C-amido, N-amido, and amino.

As used herein, the phrase “treating . . . with . . . a compound (or a composition containing a compound)” or paraphrases thereof means either administering the compound to cells or an animal, or administering to cells or an animal the compound or another agent to cause the presence or formation of the compound inside the cells or the animal. Preferably, the methods of the present invention comprise administering to cells in vitro or to a warm-blood animal, particularly mammal, more particularly a human a pharmaceutical composition comprising an effective amount of a compound according to the present invention.

As used herein, the term “preventing an increase in a symptom” refers to both not allowing a symptom to increase or worsen, as well as reducing the rate of increase in the symptom. For example, a symptom can be measured as the amount of particular disease marker, i.e., a protein. In another example the symptom can be cognitive decline. Preventing an increase, according to the definition provided herein, means that the amount of symptom (e.g., protein or cognitive decline) does not increase or that the rate at which it increases is reduced.

As used herein, the term “treating Alzheimer's disease” refers to a slowing of or a reversal of the progress of the disease. Treating Alzheimer's disease includes treating a symptom and/or reducing the symptoms of the disease.

As used herein, the term “preventing Alzheimer's disease” refers to a slowing of the disease or of the onset of the disease or the symptoms thereof. Preventing Alzheimer's disease can include stopping the onset of the disease or symptoms thereof.

As used herein, the term “Aβ₄₂ lowering” refers to the capability to reduce the amount of Aβ₄₂ present and/or being produced. Levels of Aβ₄₂ can be determined with an ELISA assay configured to detect Aβ₄₂. Methods of determining Aβ₄₂ levels are described in the examples and references cited therein.

As used herein, the term “unit dosage form” refers to a physically discrete unit, such as a capsule or tablet suitable as a unitary dosage for a human patient. Each unit contains a predetermined quantity of a compound of Formula I-Va, which was discovered or believed to produce the desired pharmacokinetic profile which yields the desired therapeutic effect. The dosage unit is composed of a compound of Formula I-Va in association with at least one pharmaceutically acceptable carrier, salt, excipient, or combination thereof.

As used herein, the term “dose” or “dosage” refers the amount of active ingredient that an individual takes or is administered at one time. For example, an 800 mg dose of a compound of Formula I-Va refers to, in the case of a twice-daily dosage regimen, a situation where the individual takes 800 mg of a compound of Formula I-Va twice a day, e.g., 800 mg in the morning and 800 mg in the evening. The 800 mg of a compound of Formula I-Va dose can be divided into two or more dosage units, e.g., two 400 mg dosage units of a compound of Formula I-Va in tablet form or two 400 mg dosage units of a compound of Formula I-Va in capsule form.

“A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

“A pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

### Dosages, Formulations, and Routes of Administration

The active compounds of this invention are typically administered in combination with a pharmaceutically acceptable carrier through any appropriate routes such as parenteral, oral, or topical administration, in a therapeutically (or prophylactically) effective amount according to the methods set forth above. A preferred route of administration for use in the invention is oral administration.

Generally, the toxicity profile and therapeutic efficacy of the therapeutic agents can be determined by standard pharmaceutical procedures in suitable cell models or animal models. As is known in the art, the LD50 represents the dose lethal to about 50% of a tested population. The ED50 is a parameter indicating the dose therapeutically effective in about 50% of a tested population. Both LD50 and ED50 can be determined in cell models and animal models. In addition, the IC50 may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED50 and/or IC50, but remains significantly below the LD50 dosage level, as determined from cell or animal models.

Typically, the compounds and compositions for use in the invention can be effective at an amount of from about 0.05 mg to about 4000 mg per day, preferably from about 0.1 mg to about 2000 mg per day. However, the amount can vary with the body weight of the patient treated and the state of disease conditions. The active ingredient may be administered at once, or may be divided into a number of smaller doses to be administered at predetermined intervals of time. The EC50 values discussed previously can desirably be used to identify specific pro-apoptotic compounds and compositions that can be used within predetermined, desirable dosage ranges.

In the case of combination therapy, a therapeutically effective amount of another therapeutic compound can be administered in a separate pharmaceutical composition, or alternatively included in the pharmaceutical composition according to the present invention. The pharmacology and toxicology of other therapeutic compositions are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in the art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

If the compound for use in the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound for use in the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These substituents may optionally be further substituted with a substituent selected from such groups.

### EXAMPLES

### Example 2

### Synthesis of Compounds

General: Chemicals were purchased from standard commercial vendors and used as received unless otherwise noted. “Degassed” means reduced pressure then nitrogen gas for three cycles. Abbreviations are consistent with those in the ACS Style Guide., plus: satd (saturated), DCM (dichloromethane), pRPLC (preparative reverse phase HPLC), “dry” glassware means oven/desiccator dried. Solvents were ACS grade unless otherwise noted. Analytical TLC plates (Silica Gel 60 F254, EM Science, Gibbstown, N.J., or Merck #5715) were used to follow the course of reactions, and the MPLC system used for purifications was from Isco (Foxy Jr fraction collector, UA-6 detector), using Isco silica gel flash columns (10 or 40 g). ¹H NMR spectra in CDCl₃, CD₃OD, and/or d6-DMSO were recorded on either a Varian Mercury 400 MHz or Brucker ARX-300 MHz instrument and chemical shifts are expressed in parts per million (ppm, δ) relative to TMS as the internal standard. Mass spectra were obtained on a Thermo Finnigan LCQ-Deca (injection volume 5 uL, XTerra MS-C₁₈ 3.5 μm 2.1×50 mm column, XTerra MS-C₁₈ 5 μm 2.1×20 mm guard column), ESI source, analytical HPLC was performed on an HP1050 (injection volume 5 μl, XTerra RP-C₁₈ 5 μm 4.6×250 mm column, with an XTerra MS-C₁₈ 5 μm 2.1×20 mm guard column), and preparative HPLC was performed on an Agilent 1100 Prep-LC with various columns and conditions depending on the compound. GCMS was performed on either an Agilent Technology 6890N or Shimadzu QP5000/17A instrument. Yields are unoptimized.

Synthetic Scheme for Compound 20

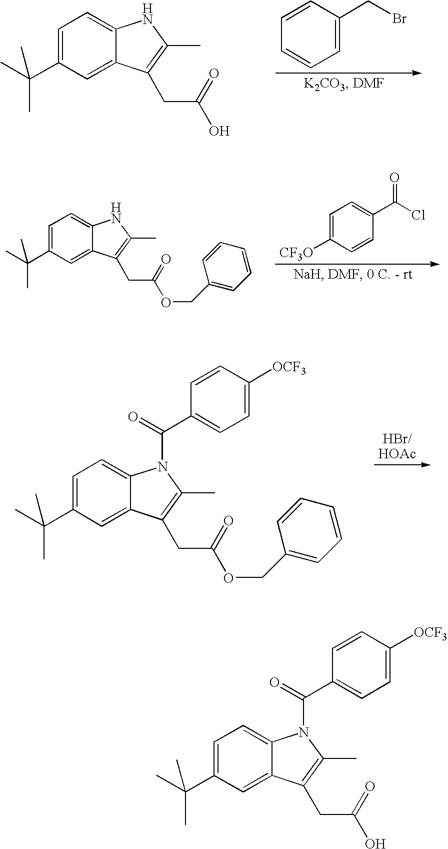

Experimental Section for the Synthesis of Compound 20

5-t-butyl-2-methylindole-3-benzylacetate: A mixture of 0.5 g (2 mmol) of 5-t-butyl-2-methylindole-3-acetic acid, 0.28 g (2 mmol) of potassium carbonate and 0.24 g (2 mmol) of benzyl bromide in 20 mL of DMF was stirred overnight at RT. The reaction mixture was diluted with 30 ml of water and extracted with CH₂Cl₂ (2×30 mL). The combined organic solutions are washed with water (2×20 mL), dried (Na₂SO₄) filtered, and the solvent removed in vacuo. The crude product was purified by MPLC (5%-10% EtOAc/hexanes as eluent) and obtained as a yellow solid. ¹H NMR (400 MHz, CDCl₃) δ 7.8 (s, 1H), 7.2-7.5 (8H), 5.1 (s, 2H), 3.7 (s, 2H), 2.4 (s, 3H), 1.4 (s, 9H); GCMS: 9.1 min RT, 335 mass.

1-(p-trifluoromethoxybenzoyl)-5-t-butyl-2-methyl-3-benzylacetate: To a solution of 0.67 g (1.99 mmol) of 5-t-butyl-2-methylindole-3-benzylacetate in dry DMF (20 mL) was added 0.095 g of NaH (2.39 mmol; 60% dispersion in mineral oil) at 0° C., under nitrogen. The reaction mixture was stirred at 0° C. for 20 min, and then 0.49 g (2.19 mmol) of 4-trifluoromethoxybenzoyl chloride in 2 mL DMF was added dropwise. The reaction mixture was then stirred at ambient temperature for 20 h, diluted with water (30 mL) and extracted with EtOAc (2×30 mL). The combined organic solutions were washed with water (2×25 mL), dried (Na₂SO₄), and filtered, and the solvent removed in vacuo. The crude product was purified by MPLC (5%-20% EtOAc/hexanes as eluent) and obtained as an oil. ¹H NMR (400 MHz, CDCl₃) δ 6.9-7.8 (12H), 5.1 (s, 2H), 3.7 (s, 2H), 2.4 (s, 3H), 1.4 (s, 9H); GCMS: 11 min RT, 523 mass.

1-(p-trifluoromethoxybenzoyl)-5-t-butyl-2-methyl-3-acetic acid: A mixture of 0.22 g (0.42 mmol) of 1-(p-trifluoromethoxybenzoyl)-5-t-butyl-2-methyl-3-benzylacetate and 12 mL of 33 wt % HBr/HOAc was stirred at 45-50° C. for 5 h. After cooling, the reaction mixture was poured into a beaker with 70 mL of water. A white precipitate appeared, and was allowed to sit for 2 h, then the precipitate was filtered off and washed with water, and then dried in vacuo. The purification of the crude product was done by preparative HPLC, and the product was obtained as white crystals. ¹H NMR (400 MHz, CDCl₃) δ 6.8-7.9 (7H), 3.7 (s, 2H), 2.4 (s, 3H), 1.4 (s, 9H); ESI (positive mode) 479 (M+2Na), ESI (negative mode) 432 (M-H).

Synthetic Scheme for Compound 40

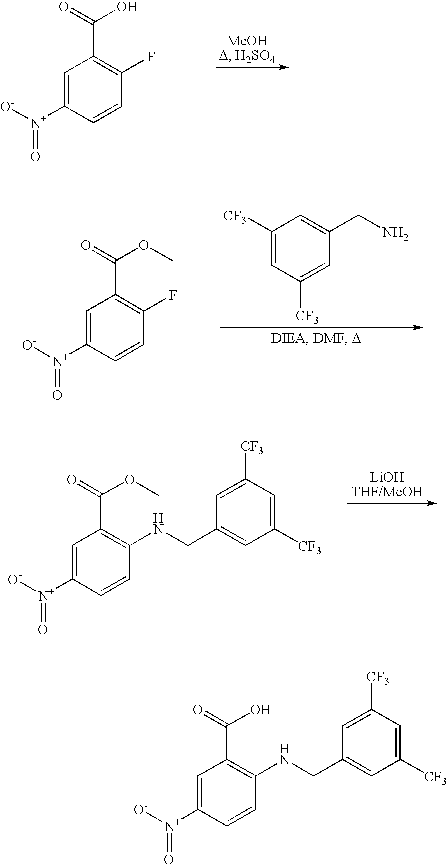

Experimental Section for Synthesis of Compound 40

2-fluoro-5-nitrobenzoic acid methyl ester: To a solution of 3.3 g (17.8 mmol) 2-fluoro-5-nitrobenzoic acid in 10 mL (246 mmol) MeOH in a 100 mL round-bottom flask with a magnetic stir bar, was added 0.25 mL (catalytic) concentrated sulfuric acid. The flask was fitted with a reflux condenser and heating mantle, and the clear yellow solution stirred at 80° C. for 7 h. After cooling, the solution was extracted from water 2×EtOAc, the organic layers combined and washed once each with 1M HCl, saturated NaHCO₃, and brine, dried over sodium sulfate, filtered and concentrated in vacuo to a pale yellow oil that solidified upon standing. ¹H (300 MHz, CDCl₃) δ 8.9 (m, 1H), 8.5 (m, 1H), 7.4 (t, 1H), 4.0 (s, 3H). GCMS: RT=4.36 min, MW=199.

2-(3,5-bis-trifluoromethylbenzylamino)-5-nitrobenzoic acid methyl ester: To a solution of 0.198 g (1.01 mmol) of 2-fluoro-5-nitrobenzoic acid methyl ester in 8.0 mL anhydrous DMF in a 25 mL round-bottomed flask with a magnetic stir bar, was added 0.695 g (2.86 mmol) 3,5-bis(trifluoromethyl)benzylamine and 0.27 mL (1.55 mmol) DIEA. The flask was fitted with a reflux condenser and heating mantle, and the yellow suspension was stirred at 80° C. for 4 h. The yellow suspension turned clear within 15 min. After cooling to room temperature, the solution was extracted from water 2×EtOAc, the organic layers combined and washed once each with water, dilute HCl, saturated NaHCO₃, and brine, dried over sodium sulfate, filtered and concentrated in vacuo to a pale yellow solid. This material was purified by MPLC using EtOAc/hexanes (10%-50% gradient), the main product eluted as a single peak on GCMS: RT=9.8 min, MW=422 MW. ¹H NMR (300 MHz, CDCl₃ δ 9.1 (s, 1H), 8.3 (s, 1H), 8.2 (d, 1H), 7.8 (s, 1H), 7.7 (s, 2H), 6.5 (d, 1H), 4.7 (d, 2H), 4.0 (s, 3H).

2-(3,5-bis-trifluoromethylbenzylamino)-5-nitrobenzoic acid: To a solution of 0.360 g (0.90 mmol) of 2-(3,5-bis-trifluoromethylbenzylamino)-5-nitrobenzoic acid methyl ester in 10.0 mL of a 3:1 mixture of THF/MeOH in a 100 mL round-bottom flask with a magnetic stir bar, was added 2.7 mL (2.7 mmol) 1.0M LiOH, to give a clear yellow solution that darkened over time. The flask was loosely capped with a rubber septum, and the solution stirred at room temperature for 8 h. The solution was extracted from 1M HCl with 2×EtOAc, the organic layers combined and washed once each with 1M HCl and brine, dried over sodium sulfate, filtered and concentrated in vacuo to a yellow solid. HPLC RT=16.9 min; LCMS (negative mode), 407 MW (M-H); ¹H NMR (400 MHz, CDCl₃/CD₃OD) δ 8.9 (s, 1H), 8.2 (dd, 1H), 7.8 (s, 1H), 7.7 (s, 2H), 6.5 (d, 1H), 4.7 (s, 2H).

Synthetic Scheme for Compound 53

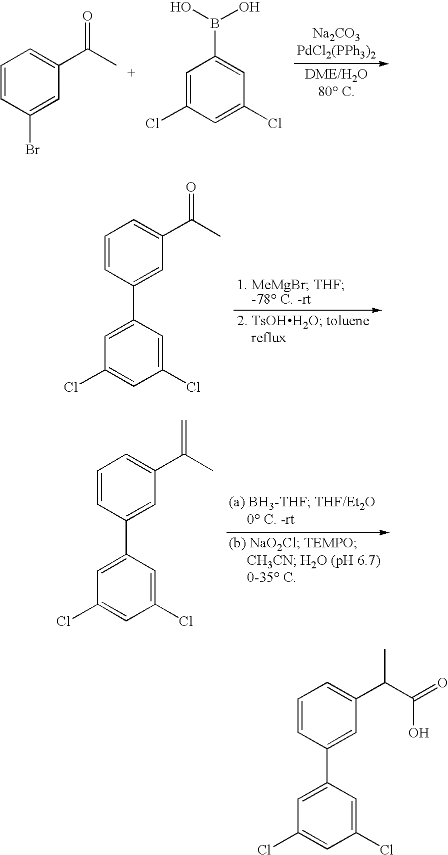

Experimental Section for Synthesis of Compound 53

Water (20 mL) and DME (100 mL) were added to a flask containing m-bromoacetophenone (3.995 g; 20.1 mmol), 3,5-dichlorobenzeneboronic acid (4.215 g; 22.1 mmol), sodium carbonate (3.195 g; 30.1 mmol) and bis(triphenylphosphine)palladium(II) chloride (423 mg; 0.603 mmol). The mixture was degassed then heated under a nitrogen atmosphere for 45 h; whereupon the organic volatiles were removed on a rotary evaporator. Water (20 mL) was added and the crude product extracted into a mixture of EtOAc (40 mL) and ether (50 mL). The organic portion was washed with 1 M NaOH (2×20 mL), 1 M HCl (2×20 mL) and satd NaCl (2×25 mL); then dried over MgSO₄, filtered and concentrated to 5.82 g of a white solid. This crude material was recrystallized from hot hexanes (300 mL) yielding 2.92 g (55%) of 1-(3′,5′-dichloro-biphenyl-3-yl)-ethanone as white crystals. R_{f} 0.22 (10:1 hexanes:EtOAc); ¹H NMR (300 MHz, CDCl₃) δ 8.12 (m, 1H), 7.98 (m, 1H), 7.74 (m, 1H), 7.57 (m, 1H), 7.49 (m, 2H), 7.38 (m, 1H).

Using dry glassware, methylmagnesium bromide (3.0 M in ether; 1.50 mL; 4.5 mmol) was added dropwise via syringe to a soln at −78° C. of the above ketone (1.000 g; 3.77 mmol) in anhydrous THF (20 mL). After 2.6 h at −78° C. and brief ambient warming, the flask was put into a rt water bath then quenched after 10 min with 1 M HCl (10 mL). The organic volatiles were removed on a rotary evaporator and the crude product extracted into toluene (15 mL). The organic portion was washed with 1 M HCl (1×10 mL) and satd NaCl (2×10 mL), then dried over MgSO₄ and filtered into a round-bottomed flask yielding crude 2-(3′,5′-dichloro-biphenyl-3-yl)-propan-2-ol. TsOH.H₂O (36 mg; 0.19 mmol) was added and the rxn was heated at reflux overnight, then concentrated on a rotary evaporator and purified by MPLC (10 g SiO₂ with hexanes as eluant) yielding 600 mg of 3,5-dichloro-3′-isopropenyl-biphenyl as a clear, colorless liquid (60% over two steps). R_{f} 0.52 (hexanes); GC-MS (t_{R}=7.0 min; m/z 262 [M]⁺).

Using dry glassware, BH₃.THF (1.5 M in THF/ether) was added dropwise to a 0° C. soln of the above styrene (600 mg; 2.28 mmol) in anhydrous THF. After 1.2 h at 0° C., potassium phosphate buffer (0.67 M; pH 6.7) was added (cautiously at first). The organic volatiles were removed on a rotary evaporator then acetonitrile (15 mL), TEMPO (25 mg; 0.16 mmol) and sodium chlorite (tech=80 wt %; 1.097 g; 9.7 mmol) were added. The rxn was heated at 35° C. for 40 h with vigorous stirring, then cooled in an ice-water bath and carefully quenched with sodium sulfite (428 mg; 3.4 mmol) and the pH adjusted to ca. 9, stirred for a short while then acidified with concentrated HCl. Water was added and the crude product extracted into DCM. The soln was dried over MgSO₄, filtered and concentrated. 2-(3′,5′-Dichloro-biphenyl-3-yl)-propionic acid was partially purified by MPLC (SiO₂/0-50% EtOAc in hexanes) and further purified by pRPLC (149 mg; 22%). ¹H NMR (300 MHz, CDCl₃) δ 7.5-7.3 (m, 7H), 3.82 (q, J=7.2 Hz, 1H), 1.57 (d, J=7.2 Hz, 3H); HPLC (t_{R}=16.9 min); LC-MS (t_{R}=8.9 min; m/z 293([M−1]⁻; ESI⁻).

Synthetic Scheme for Compound 55

Experimental Section for Synthesis of Compound 55

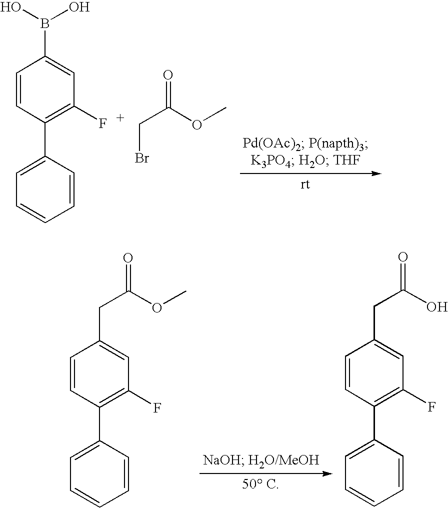

THF (40 mL), water (0.38 mL; 21.1 mmol) and methyl bromoacetate (1.0 mL; 10.5 mmol) were added to a mixture of Pd(OAc)₂ (67.2 mg; 0.299 mmol), tri(1-naphthyl)phosphine (369 mg; 0.895 mmol), potassium phosphate (10.614 g; 50.0 mmol) and 2-fluoro-biphenyl-4-boronic acid (2.593 g; 12.0 mmol). The rxn was degassed then vigorously stirred at rt. After 24 h, EtOAc (125 mL) was added and the mixture washed with water (3×25 mL) and satd NaCl (3×25 mL); then dried over MgSO₄, filtered, adsorbed onto silica then purified by MPLC (120 g SiO₂/0-20% EtOAc in hexanes yielding 1.527 g of impure (2-fluoro-biphenyl-4-yl)-acetic acid methyl ester (ca. 82 wt % by GC-MS; ca. 49% yield). ¹H NMR (300 MHz, CDCl₃) δ 7.6-7.3 (m, 6H), 7.2-7.0 (m, 2H), 3.73 (s, 3H), 3.67 (s, 2H); GC-MS (t_{R}=6.3 min; m/z 244([M⁺]).

The above methyl ester (127 mg; 0.520 mmol), 1 M NaOH (1 mL) and MeOH (1 mL) were heated at 50° C. After 16.5 h the reaction was acidified with 1 M HCl (5 mL), the organic volatiles removed on a rotary evaporator then the product extracted into EtOAc (5 mL). The organic portion was washed with 1 M HCl (3×2 mL) and satd NaCl (2×2 mL), dried over MgSO₄, filtered then purified by MPLC (12 g SiO₂/EtOAc in hexanes gradient) yielding (2-fluoro-biphenyl-4-yl)-acetic acid as a white solid. ¹H NMR (300 MHz, CDCl₃) δ 7.6-7.5 (m, 2H), 7.5-7.3 (m, 4H), 7.2-7.1 (m, 2H), 3.70 (s, 2H); HPLC (t_{R}=11.7 min); LC-MS (t_{R}=5.3 min; m/z 229 ([M−1]⁻; ESI−)); GC-MS (t_{R}=6.9 min; m/z 230 ([M⁺]).

Synthetic Scheme for Compound 56

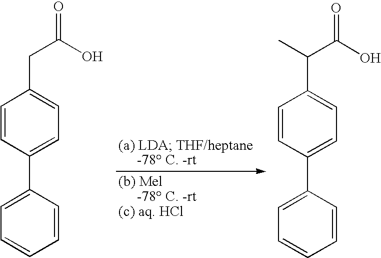

Experimental Section for Synthesis of Compound 56

Using dry glassware, LDA (2 M in THF/heptane; 2.0 mL; 4.0 mmol) was added to a −78° C. soln of biphenyl-4-yl-acetic acid (387 mg; 1.82 mmol) in anhydrous THF (4 mL). THF (15 mL) was added to the resulting ppt and the rxn warmed to rt to try to dissolve the ppt. The rxn was cooled to −78° C., neat CH₃I (227 μL; 3.64 mmol) was added then the rxn stirred at rt. After 16 h the rxn was quenched with 1 M HCl (5 mL), the organic volatiles removed on a rotary evaporator then the product extracted into EtOAc (5 mL). The org. soln was washed with 1 M HCl (3×2 mL) and satd NaCl (2×2 mL), dried over MgSO₄, filtered then purified by MPLC (12 g SiO₂/EtOAc in hexanes gradient) yielding 95 mg of 2-biphenyl-4-yl-propionic acid as a solid (23%). ¹H NMR (300 MHz, CDCl₃) δ 7.6-7.5 (m, 4H), 7.5-7.3 (m, 5H), 3.80 (q, J=7.2 Hz, 1H), 1.56 (d, J=7.2 Hz, 3H); HPLC (t_{R}-=12.4 min); LC-MS (t_{R}=5.45 min; m/z 225 ([M−1]⁻; (ESI−)).

Synthetic Scheme for Compound 57

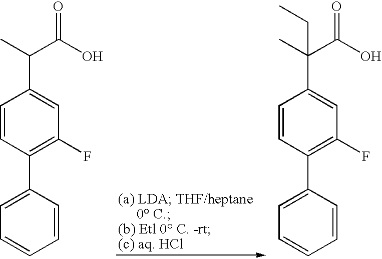

Experimental Section for Synthesis of Compound 57

LDA (2 M in THF/heptane; 0.75 mL; 1.5 mmol) was added to a 0° C. soln of 2-(2-fluoro-biphenyl-4-yl)-propionic acid (150 mg; 0.614 mmol) in anhydrous THF (5 mL). Neat iodoethane (99 μL; 1.2 mmol) was added after 10 min and the rxn was allowed to warm to rt. After 20 h, the rxn was concentrated on a rotary evaporator, 1 M HCl (3 mL) was added then the product extracted into EtOAc (5 mL). The organic portion was washed with 1 M HCl (2 mL) and hexanes (2 mL) was added to facilitate separation of the layers. The soln was further washed with satd NaCl (3 mL), filtered through a plug of silica then purified by MPLC (12 g SiO2/0-30% EtOAc in hexanes) yielding 137 mg of 2-(2-fluoro-biphenyl-4-yl)-2-methyl-butyric acid as a tan, crystalline solid (82%). Rf 0.35 (2:1 hexanes:EtOAc); ¹H NMR (300 MHz, CDCl₃) δ 7.6-7.1 (m, 8H), 2.12 (m, 1H), 2.03 (m, 1H), 1.60 (s, 3H), 0.90 (app t, J=7.4 Hz, 3H); HPLC (tR=14.4 min); LC-MS (tR=6.1 min; m/z 272 ([M−1).

Synthetic Scheme for Compound 58 and Compound 63

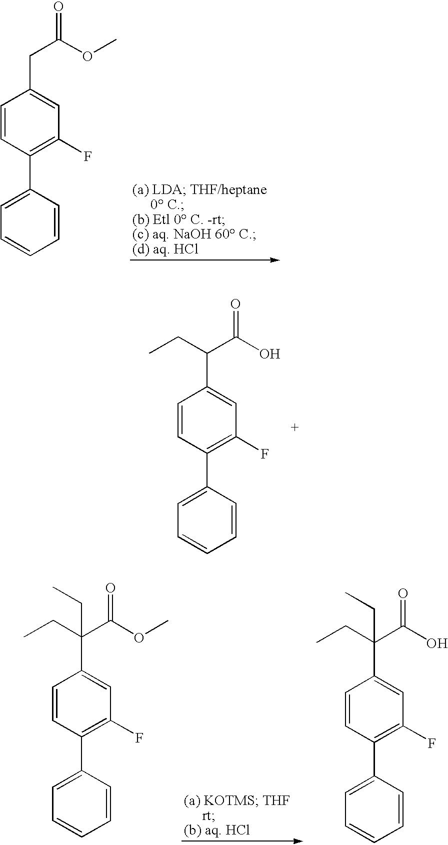

Experimental Section for Synthesis of Compound 58 and Compound 63

(2-Fluoro-biphenyl-4-yl)-acetic acid methyl ester (ca. 82 wt %; 150 mg; 0.504 mmol) was alkylated as for 2-(2-fluoro-biphenyl-4-yl)-2-methyl-butyric acid (compound 57) using THF (5 mL), LDA (2 M in THF/heptane; 0.75 mL; 1.5 mmol) and iodoethane (99 μL; 1.2 mmol). After 19 h, 1 M NaOH (2.0 mL; 2.0 mmol) was added and the rxn heated at 60° C. for 7.5 h; whereupon the organic volatiles were removed on a rotary evaporator, 2 M HCl (3 mL) was added and the products extracted into EtOAc (5 mL). This was washed with 1 M HCl (2×2 mL) and satd NaCl (2×2 mL), dried over MgSO₄, filtered through a plug of silica then purified by MPLC (12 g SiO₂/0-30% EtOAc in hexanes) yielding 60 mg (46%) of 2-(2-fluoro-biphenyl-4-yl)-butyric acid as a light orange waxy solid and 64 mg (42%) of 2-ethyl-2-(2-fluoro-biphenyl-4-yl)-butyric acid methyl ester as a pale yellow viscous liquid. 2-(2-Fluoro-biphenyl-4-yl)-butyric acid: ¹H NMR (300 MHz, CDCl₃) δ 7.6-7.5 (m, 2H), 7.5-7.3 (m, 4H), 7.2-7.1 (m, 2H), 3.51 (app t, J=7.7 Hz, 1H), 2.12 (m, 1H), 1.88 (m, 1H), 0.96 (app t, J=7.4 Hz, 3H); GC-MS (t_{R}=7.3 min; m/z 258 ([M⁺]); HPLC (t_{R}=13.7 min); LC-MS (t_{R}=6.9 min; m/z 214 ([M-CO₂H]⁻). 2-Ethyl-2-(2-fluoro-biphenyl-4-yl)-butyric acid methyl ester: ¹H NMR (300 MHz, CDCl₃) δ 7.6-7.5 (m, 2H), 7.5-7.3 (m, 4H), 7.2-7.0 (m, 2H), 3.69 (s, 3H), 2.07 (m, 4H), 0.77 (app t, J=7.4 Hz, 6H).

Potassium silanolate (90% tech; 588 mg; 4.1 mmol) was added to a soln of 2-ethyl-2-(2-fluoro-biphenyl-4-yl)-butyric acid methyl ester (62 mg; 0.21 mmol) in anhydrous THF (4.2 mL). After 2 days at rt the rxn was determined to be incomplete by TLC and the temperature was increased to 60° C. After 15 days at 60° C. the rxn was cooled to rt, quenched with 2 M HCl (2.5 mL) then the organic volatiles were removed on a rotary evaporator. The product was extracted into EtOAc (5 mL), washed with satd NaCl (1×4 mL), dried over MgSO₄, filtered through a plug of silica then conc on a rotary evaporator. Pure 2-ethyl-2-(2-fluoro-biphenyl-4-yl)-butyric acid (47 mg; 80%) was obtained as a white crystalline solid after MPLC (12 g SiO₂/0-40% EtOAc in hexanes). ¹H NMR (300 MHz, CDCl₃) δ 7.6-7.5 (m, 2H), 7.5-7.3 (m, 4H), 7.2-7.0 (m, 2H), 2.10 (m, 4H), 0.82 (app t, J=7.4 Hz, 6H); GC-MS (t_{R}=8.2 min; m/z 286 ([M⁺]); HPLC (t_{R}=16.7 min); LC-MS (t_{R}=8.7 min; m/z 285 (M−1).

Synthetic Scheme for Compound 59

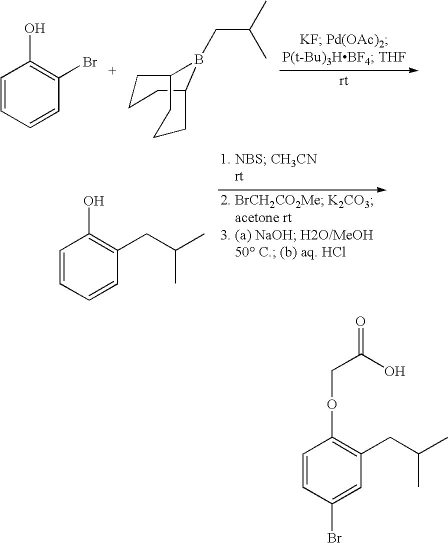

Experimental Section for Synthesis of Compound 59

In dry glassware, isobutylene gas was bubbled for 10 min into a soln of 9-BBN-H (0.5 M in THF; 30.0 mL; 15.0 mmol). The rxn was degassed. 2-Bromophenol (1.50 mL; 12.9 mmol), KF (2.256 g; 38.8 mmol), Pd(OAc)₂ (87.1 mg; 0.388 mmol) and P(t-Bu)₃H.BF₄ (113 mg; 0.388 mmol) were added and the rxn again degassed. After 23 h the rxn was concentrated on a rotary evaporator. Ether (50 mL) was added and washed with water (1×15 mL), 1 M HCl (2×15 mL), 1 M NaOH (3×15 mL) and satd NaCl (2×15 mL). The soln was dried over MgSO₄, filtered, adsorbed onto silica then purified by MPLC (40 g SiO₂/1-10% EtOAc in hexanes) yielding 1.836 g of 2-isobutyl-phenol as a pale yellow liquid that was 81 wt % pure by GC-MS (77%). ¹H NMR (300 MHz, CDCl₃) δ 7.1-7.0 (m, 2H), 6.86 (m, 1H), 6.76 (m, 1H), 4.58 (s, 1H), 2.48 (d, J=7.2 Hz, 2H), 1.93 (m, 1H), 0.93 (d, J=6.6 Hz, 6H); GC-MS (t_{R}=2.8 min; m/z 150 ([M⁺]).

Solid NBS (1.771 g; 9.95 mmol) was added in one portion to a soln of the above phenol (1.83 g; 9.89 mmol) in acetonitrile at rt. After 50 min the rxn mixture was adsorbed onto silica then purified by MPLC (40 g SiO₂/0-10% EtOAc in hexanes) yielding 2.21 g of 4-bromo-2-isobutyl-phenol as a tan liquid (92 wt % pure by GC-MS; 90%). ¹H NMR (300 MHz, CDCl₃) δ 7.2-7.1 (m, 2H), 6.64 (d, J=8.2 Hz, 1H), 4.60 (s, 1H), 2.44 (d, J=7.2 Hz, 2H), 1.92 (m, 1H), 0.93 (d, J=6.6 Hz, 6H).

Methyl bromoacetate (1.25 mL; 13.2 mmol) was added to a suspension of potassium carbonate (1.84 g; 13.3 mmol) and the above bromophenol (2.03 g; 8.2 mmol) in acetone (15 mL). After 44 h at rt the rxn was conc on a rotary evaporator. Ether (20 mL) was added and washed with water (1×8 mL then 1×5 mL), 1 M HCl (1×5 mL) and satd NaCl (2×5 mL). After drying over MgSO₄ and filtration, the crude product was adsorbed onto silica then purified by MPLC (40 g SiO₂/0-100% EtOAc in hexanes) yielding pure (4-bromo-2-isobutyl-phenoxy)-acetic acid methyl ester as a light tan liquid (2.444 g; 100%). GC-MS (t_{R}=5.9 min; m/z 300/302 ([M⁺]).

The above methyl ester (155 mg; 0.515 mmol) was saponified in an analogous manner as for (2-fluoro-biphenyl-4-yl)-acetic acid methyl ester (compound 55) and purified by pRPLC yielding (4-bromo-2-isobutyl-phenoxy)-acetic acid as a white solid. ¹H NMR (300 MHz, CDCl₃) δ 7.3-7.2 (m, 2H), 6.61 (m, 1H), 4.66 (s, 2H), 2.50 (d, J=7.1 Hz, 2H), 1.92 (m, 1H), 0.91 (d, J=6.6 Hz, 6H); HPLC (t_{R}=14.3 min); LC-MS (t_{R}=7.2 min; m/z 287 ([M−1]⁻).

Synthetic Scheme for Compound 60

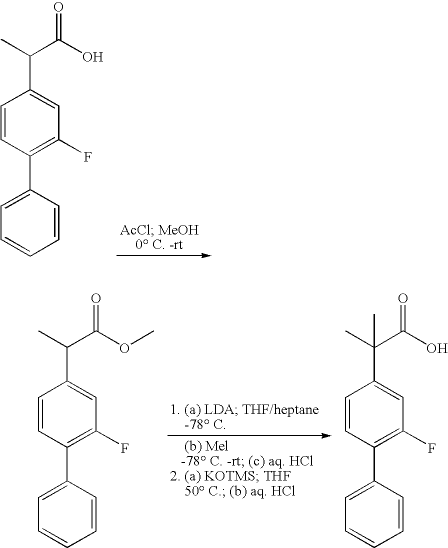

Experimental Section for Synthesis of Compound 60

Neat acetyl chloride (0.90 mL; 12.7 mmol) was added to dry methanol (25 mL) at 0° C. After warming to rt over 10 min, solid (R)-flurbiprofen (6.109 g; 25.0 mmol) was added. The reaction was concentrated on a rotary evaporator after 26 h. The resulting oil was dissolved in ethyl acetate (40 mL) then washed with 1 M NaOH (1×10 mL), 1 M HCl (1×10 mL) and saturated NaCl (1×10 mL). The organic portion was dried (MgSO₄), filtered and concentrated to give 6.3 g of (R)-2-(2-fluoro-biphenyl-4-yl)-propionic acid methyl ester as a clear, colorless liquid (98%). ¹H NMR (300 MHz, CDCl₃) δ 7.55-7.48 (m, 2H), 7.48-7.30 (m, 4H), 7.18-7.05 (m, 2H), 3.76 (q, J=7.1 Hz, 1H), 3.70 (s, 3H), 1.54 (d, J=7.1 Hz, 3H); GCMS (t_{R}=6.4 min, m/z 258 (M⁺)).

A solution of (R)-2-(2-fluoro-biphenyl-4-yl)-propionic acid methyl ester (1.943 g; 7.52 mmol) in dry THF (8 mL) was added over ca. 4 min via syringe to a solution at −78° C. of LDA (4.5 mL of 2.0M; 9.0 mmol) in heptane/THF. THF (2 mL) was used to quantitatively transfer the ester and THF (20 mL) was added to the resulting precipitate. The reaction was warmed in a rt water bath to dissolve the precipitate, then neat iodomethane (0.50 mL; 8.0 mmol) was added. After 3.0 b the reaction was quenched with 1M HCl (10 mL) then the organic volatiles were removed on a rotary evaporator. The product was extracted into ethyl acetate (25 mL) and the organic portion was washed with 1M HCl (3×10 mL), saturated NaHCO₃ (2×10 mL), saturated NaCl (2×10 mL), then dried (MgSO₄), filtered and concentrated. 2-(2-Fluoro-biphenyl-4-yl)-2-methyl-propionic acid methyl ester was purified by MPLC (40 g SiO₂ column, 0-10% EtOAc/hexanes) to a clear, colorless oil which solidified to a waxy solid. This material was 12.2:1 product:starting material (93 wt % pure) by GC-MS and was used as is for the following reaction. ¹H NMR (300 MHz, CDCl₃) δ 7.58-7.49 (m, 2H), 7.49-7.31 (m, 4H), 7.20-7.08 (m, 2H), 3.70 (s, 3H), 1.61 (s, 6H); GCMS (t_{R}=6.6 min. m/z 272 (M⁺)).

Solid KOTMS (6.34 g; 44.5 mmol) was added to a solution of the above methyl ester (1.211 g; 4.13 mmol) in dry THF (25 mL). The reaction was put under an atmosphere of nitrogen then heated at 50° C. for 20 h, then cooled to 0° C. and acidified with concentrated HCl (5 mL). After concentration on a rotary evaporator, EtOAc (25 mL) was added and washed with water (1×15 mL then 2×5 mL) and saturated NaCl (2×8 mL). The solution was dried (MgSO₄), filtered, concentrated then purified by pRPLC yielding 153 mg of 2-(2-fluoro-biphenyl-4-yl)-2-methyl-propionic acid as a white solid (14% yield). ¹H NMR (400 MHz, CDCl₃) δ 7.56-7.51 (m, 2H), 7.47-7.34 (m, 4H), 7.28-7.19 (m, 2H), 1.64 (s, 6H); HPLC (t_{R}=13.5 min); LC-MS (t_{R}=6.9 min; m/z 214 ([M-CO₂H]⁻).

Synthetic Scheme for Compound 61

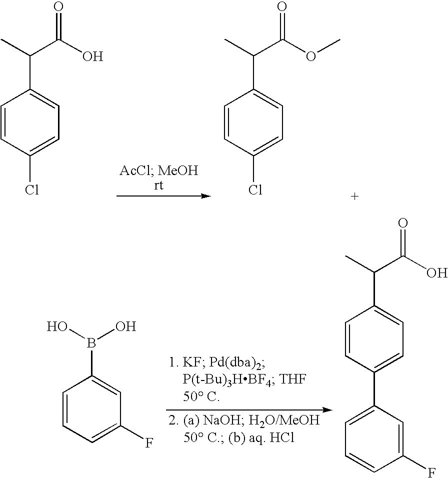

Experimental Section for Synthesis of Compound 61

2-(4-Chloro-phenyl)-propionic acid methyl ester was synthesized in an analogous manner as for (R)-2-(2-fluoro-biphenyl-4-yl)-propionic acid methyl ester (compound 60) from 2-(4-chloro-phenyl)-propionic acid (4.000 g; 21.7 mmol), acetyl chloride (1.5 mL; 21.1 mmol) and methanol (35 mL) yielding 3.986 g of a light yellow liquid after MPLC purification (120 g SiO₂/EtOAc in hexanes gradient). GC-MS (t_{R}=3.5 min; m/z 198 ([M⁺]).

Anhydrous THF (2.0 mL) was added to a vial containing the above ester (149 mg; 0.735 mmol), 3-fluorophenylboronic acid (115 mg; 0.822 mmol), potassium fluoride (141 mg; 2.43 mmol), Pd(dba)₂ (14.5 mg; 0.0252 mmol) and P(t-Bu)₃H.BF₄ (8.9 mg; 0.031 mmol). The rxn was degassed then heated at 50° C. for 44 h. Hexanes (2 mL) was added and the rxn filtered through a plug of silica and washed through with EtOAc. Concentration on a rotary evaporator yielded crude 2-(3′-fluoro-biphenyl-4-yl)-propionic acid methyl ester, which was used as is in the next rxn. R_{f} 0.26 (10:1 hexanes:EtOAc).

The above methyl ester was saponified in an analogous manner as for (2-fluoro-biphenyl-4-yl)-acetic acid methyl ester (compound 55) and purified by MPLC (12 g SiO₂/0-50% EtOAc in hexanes) yielding 2-(3′-fluoro-biphenyl-4-yl)-propionic acid as a solid. ¹H NMR (300 MHz, CDCl₃) δ 7.54 (m, 2H), 7.5-7.2 (m, 5H), 7.03 (m, 1H), 3.80 (q, J=7.1 Hz, 1H), 1.56 (d, J=7.2 Hz, 3H); HPLC (t_{R}=14.6 min); LC-MS (t_{R}=6.9 min; m/z 200 ([M-CO₂H]⁻; ESI−)).

Synthetic Scheme for Compound 62

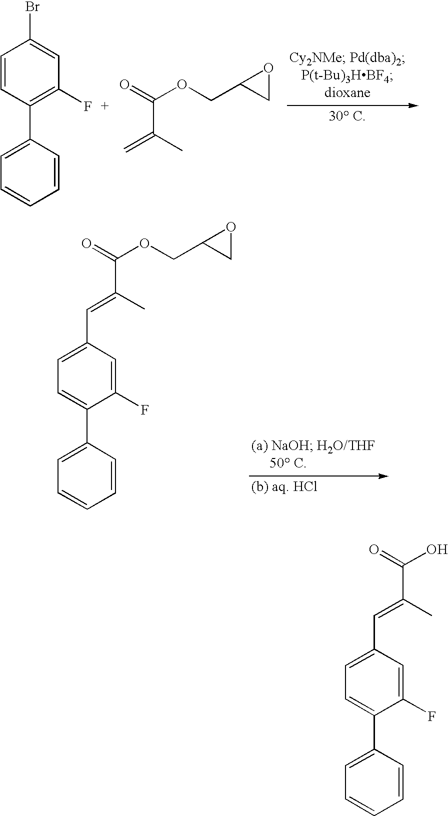

Experimental Section for Synthesis of Compound 62

Anhydrous dioxane (2.0 mL) then dicyclohexyl-methyl-amine (0.48 mL; 2.2 mmol) then 2-methyl-acrylic acid oxiranylmethyl ester (0.55 mL; 4.0 mmol) were added to the solid reagents 4-bromo-2-fluoro-biphenyl (504 mg; 2.01 mmol), Pd(dba)₂ (35 mg; 0.061 mmol) and P(t-Bu)₃H.BF₄ (17.2 mg; 0.0593 mmol). The rxn was degassed then heated at 30° C. After 94 h, EtOAc (6 mL) was added, the rxn filtered through a plug of silica, concentrated on a rotary evaporator then purified by MPLC (40 g SiO₂/0-20% EtOAc in hexanes) yielding 605 mg of (E)-3-(2-fluoro-biphenyl-4-yl)-2-methyl-acrylic acid oxiranylmethyl ester as a white solid (97%). R_{f} 0.23 (4:1 hexanes:EtOAc); ¹H NMR (300 MHz, CDCl₃) δ 7.71 (m, 1H), 7.58 (m, 2H), 7.6-7.2 (m, 6H), 4.59 (dd, J=3.0, 12.3 Hz, 1H), 4.07 (dd, J=6.4, 12.3 Hz, 1H), 3.33 (m, 1H), 2.91 (m, 1H), 2.72 (dd, J=2.6, 4.8 Hz, 1H), 2.20 (d, J=1.4 Hz, 3H); GC-MS (t_{R}=9.2 min; m/z 312 ([M⁺]).

The above ester (413 mg; 1.32 mmol), 1 M NaOH (3.0 mL) and THF (3.0 mL) were reacted at 50° C. for 70 h; whereupon the rxn was concentrated on a rotary evaporator, acidified with 1 M HCl (4 mL) then extracted with EtOAc. The organic portion was washed with satd NaCl, dried over MgSO₄, filtered through a plug of silica then purified by MPLC (40 g SiO₂/0-50% EtOAc in hexanes) yielding 83 mg of (E)-3-(2-fluoro-biphenyl-4-yl)-2-methyl-acrylic acid as a white crystalline solid (24%). R_{f} 0.23 (1:1 hexanes:EtOAc); ¹H NMR (300 MHz, CDCl₃) δ 7.80 (m, 1H), 7.59 (m, 2H), 7.6-7.2 (m, 6H), 2.21 (d, J=1.3 Hz, 3H); GC-MS (t_{R}=8.1 min; m/z 256 ([M⁺])); HPLC (t_{R}=15.8 min); LC-MS (t_{R}=7.5 min; m/z 255 ([M−1]⁻).

Synthetic Scheme for Compound 89 and Compound 90

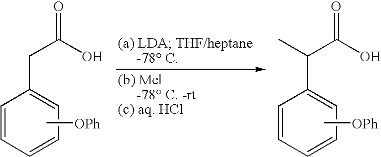

Experimental Section for Synthesis of Compound 89

2-(2-Phenoxy-phenyl)-propionic acid was synthesized in an analogous manner as for 2-biphenyl-4-yl-propionic acid (compound 55) from (2-phenoxy-phenyl)-acetic acid (327 mg; 1.43 mmol), LDA (2.0 M in heptane/THF/ethylbenzene; 1.50 mL; 3.0 mmol) and iodomethane (0.9 mL; 14.5 mmol) yielding 97 mg of pure product after purification by pRPLC (28%). ¹H NMR (300 MHz, CDCl₃) δ 7.4-6.8 (m, 9H), 4.11 (q, J=7.2 Hz, 1H), 1.50 (d, J=7.2 Hz, 3H); HPLC (t_{R}=12.2 min); LC-MS (t_{R}=5.7 min; m/z 241 ([M−1]⁻).

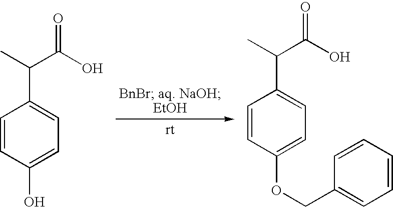

Experimental Section for Synthesis of Compound 90

2-(4-Phenoxy-phenyl)-propionic acid was synthesized in an analogous manner as for 2-biphenyl-4-yl-propionic acid (compound 56) from (4-phenoxy-phenyl)-acetic acid (327 mg; 1.43 mmol), LDA (2.0 M in heptane/THF/ethylbenzene; 1.50 mL; 3.0 mmol) and iodomethane (0.9 mL; 14.5 mmol) yielding 19 mg of pure product after purification by pRPLC (5%). ¹H NMR (300 MHz, CDCl₃) δ 7.4-6.9 (m, 9H), 3.73 (q, J=7.2 Hz, 1H), 1.52 (d, J=7.2 Hz, 3H); HPLC (t_{R}=12.5 min); LC-MS (t_{R}=5.8 min; m/z 241 ([M−1]⁻).

Synthetic Scheme for Compound 91

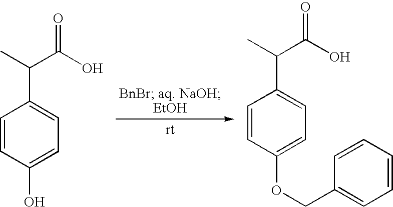

Experimental Section for Synthesis of Compound 91

A soln of 2-(4-hydroxy-phenyl)-propionic acid (335 mg; 2.02 mmol), benzyl bromide (0.26 mL; 2.2 mmol), 1 M NaOH (6 mL; 6 mmol) and 95% ethanol (20 mL) was stirred at rt. After 20 h, the organic volatiles were removed on a rotary evaporator. The rxn was acidified with 1 M HCl (10 mL) then extracted with EtOAc (15 mL). The organic portion was washed with water (1×5 mL) and satd NaCl (2×5 mL), then dried over MgSO₄, filtered and concentrated to a white solid. The material was purified by flash chromatography (50 mL SiO₂/2:1 hexanes:EtOAc) yielding 308 mg (60%) of 2-(4-benzyloxy-phenyl)-propionic acid as a white solid. Rf 0.20 (2:1 hexanes:EtOAc); ¹H NMR (300 MHz, CDCl₃) δ 7.5-7.2 (m, 7H), 6.94 (m, 2H), 5.05 (s, 2H), 3.70 (q, J=7.2 Hz, 1H), 1.49 (d, J=7.2 Hz, 3H); HPLC (t_{R}=12.5 min); LC-MS (t_{R}=5.6 min; m/z 255 ([M−1]⁻; (ESI−)).

Synthetic Scheme for Compound 92

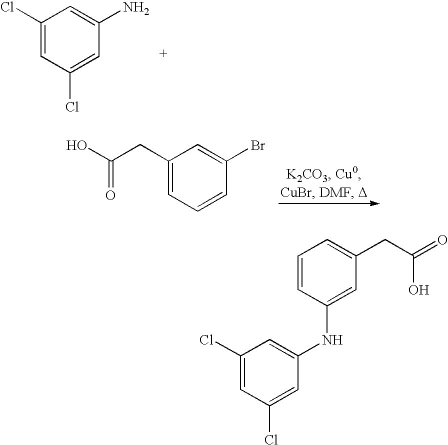

Experimental Section for Synthesis of Compound 92

[3-(3,5-dichloro-phenylamino)-phenyl]acetic acid: In a 100 mL round-bottomed flask, 3,5-dichloroaniline (3.84 g, 23.72 mmol), 3-bromophenyl acetic acid (3.00 g, 13.95 mmol), K₂CO₃ (granular, anhydrous; 3.29 g, 23.72 mmol), copper powder (50 mg, catalytic amount), and DMF (20 mL) were added and refluxed for 15 min. At this point copper bromide (3×50 mg, catalytic amount) was added over 30 min. Finally the reaction was refluxed for 4 h, then cooled to room temperature and poured into water (50 mL) and made acidic (pH 3) with HCl (12N) and extracted with EtOAc (2×25 mL). The organic layer was evaporated under vacuum to yield 928 mg (23% yield) of crude product. This material was further purified by preparatory HPLC to yield 150 mg (4% yield) of a light gray solid final product. TLC (10% MeOH in DCM) Rf=0.23. HPLC RT=6.39; MS, 295 (M+1) 250, 252, 294, 296. ¹H NMR (400 MHz, CDCl₃) δ 3.62 (s, 2H), 6.83-7.32 (m, 7H).

Generic Synthetic Schemes for Compounds 96-111:

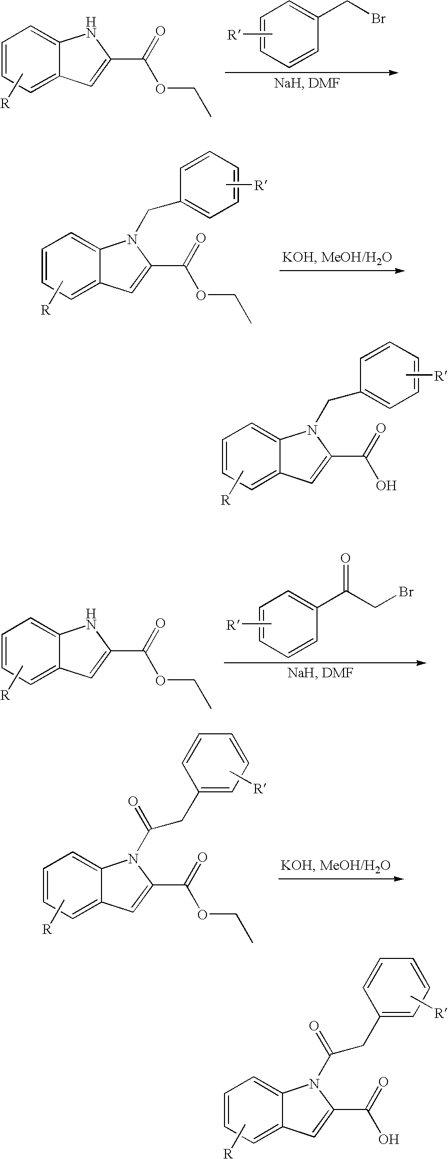

General Experimental for Compound 97:

1-(4-trifluoromethylbenzyl)-1H-indole-2-carboxylic acid ethyl ester: To a solution of 1.5 g (7.92 mmol) of ethyl indole-2-carboxylate in dry DMF (20 mL) was added 0.38 g of NaH (9.50 mmol, 60% dispersion in mineral oil) at 0° C., under nitrogen. The reaction mixture was stirred at 0° C. for 20 min, and then 2.08 g (8.70 mmol) of 4-trifluoromethylbenzyl bromide in 3 mL DMF was added dropwise. The reaction mixture was stirred at ambient temperature for 20 h, diluted with water (30 mL) and extracted with EtOAc (2×30 mL). The combined organic solutions were washed with water (2×25 mL), dried (Na₂SO₄), and filtered, and the solvent removed in vacuo. The crude product was purified by MPLC (5%-20% EtOAc/hexanes as eluent) and obtained as white crystals. ¹H NMR (400 MHz, CDCl₃); δ 7.9-7.1 (9H, ArH), 5.9 (2H, CH₂), 4.3 (2H, CH₂), 1.4 (3H, CH₃).

1-(4-trifluoromethylbenzyl)-1H-indole-2-carboxylic acid: A mixture of 1.5 g (1.43 mmol) of 1-(4-trifluoromethylbenzyl)-1H-indole-2-carboxylic acid ethyl ester and 0.081 g (1.44 mmol) of KOH in 12 mL MeOH with 4 mL H₂O, was refluxed for 4 h. After cooling, the reaction mixture was acidified with 1M HCl to a pH of 3-4, followed by extraction with EtOAc (2×30 mL). The organic layers were washed with water (2×20 mL), then brine, dried over Na₂SO₄, and concentrated in vacuo. Purification was performed by preparative TLC (5% MeOH/CH₂Cl₂ as eluant). The product was obtained as white crystals. ¹H NMR (400 MHz, DMSO-d₆); δ 7.8-7.1 (9H, ArH), 5.9 (2H, CH₂). LC/MS: neg. mode 318.05 (M-H).

Other compounds in the following Table 1 can be synthesized in a similar manner as will be apparent to skilled artisans, particularly in view of the starting reagents provided in Table 1.

TABLE 1

| Compound | | Starting Reagent 1 | Starting Reagent 2 |
|---|---|---|---|
| Number | Structure | (1 equivalent used) | (1.1 equivalent used) |
| 97 | 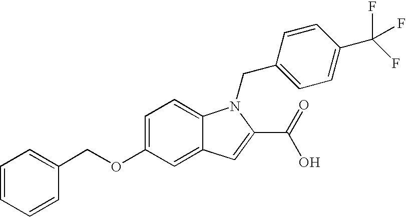 | 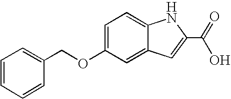 | 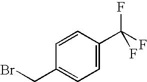 |
| 98 | 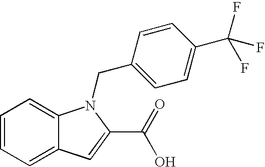 | 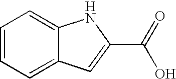 | 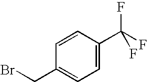 |
| 99 | 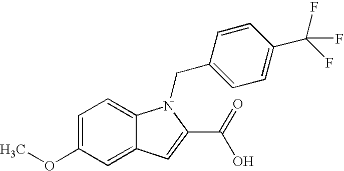 | 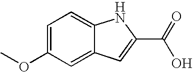 | 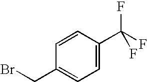 |
| 100 | 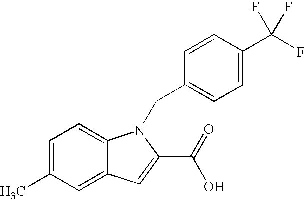 | 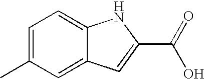 | 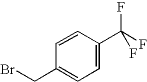 |
| 101 | 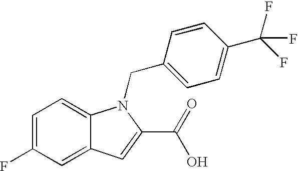 | 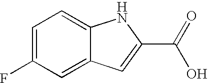 | 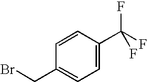 |
| 102 | 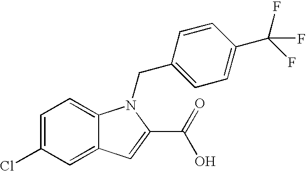 | 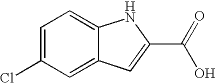 | 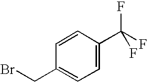 |
| 103 | 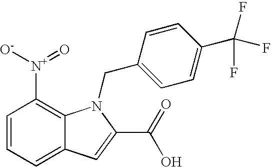 | 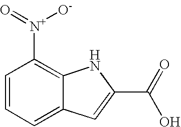 | 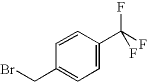 |
| 104 | 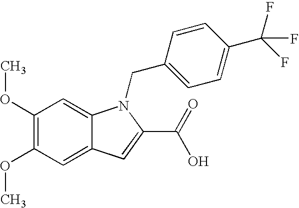 | 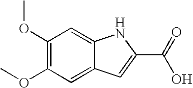 | 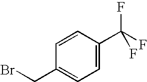 |
| 105 | 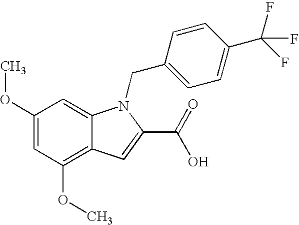 | 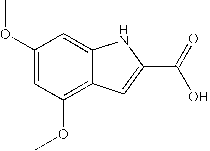 | 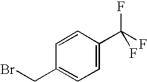 |
| 106 | 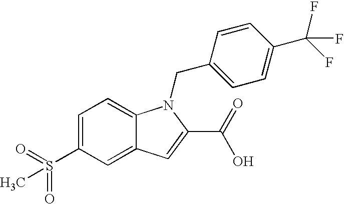 | 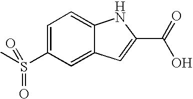 | 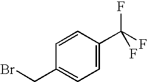 |
| 107 | 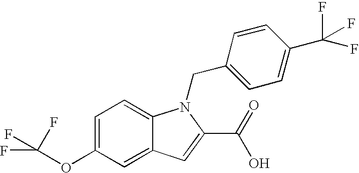 | 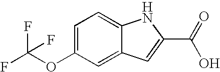 | 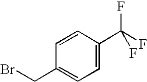 |
| 108 | 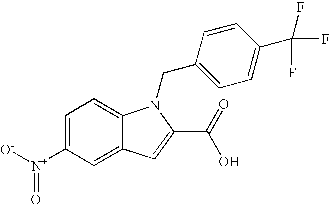 | 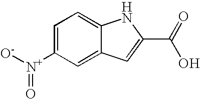 | 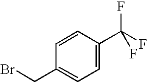 |
| 109 | 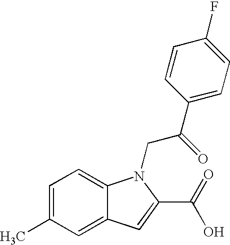 | 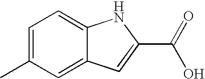 | 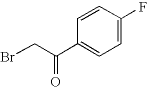 |
| 110 | 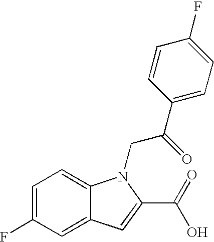 | 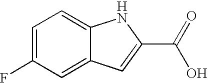 | 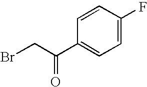 |
| 111 | 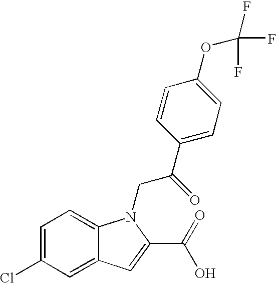 | 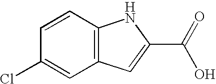 | 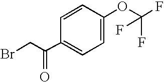 |
| 112 | 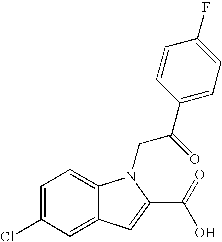 | 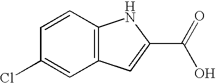 | 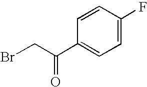 |

### Example 2

### Exemplary Compounds of the Invention

The invention is related to the inventors' discovery that compounds of Formula I-Va lower Aβ₄₂ levels in APP processing assays. Furthermore, compounds of Formula I-Va, in general, have negligible levels of COX inhibition and therefore are thought to essentially be devoid of the deleterious side-effects associated with COX inhibition. Thus, a preferred embodiment of the invention is the use of a pharmaceutical composition having one or more compounds of Formula I-Va, where the compound lowers Aβ₄₂ levels and does not substantial inhibit the cyclooxygenases. Preferred compounds of Formula I-Va for use in the invention are those that have little or negligible COX1 and/or COX2 inhibition at 1 μM, more preferred are those that have little or negligible COX1 and/or COX2 inhibition at 10 μM, and more preferred are those that have little or negligible COX1 and/or COX2 inhibition at 100 μM compound. COX1 and COX2 inhibition can be determined with a COX inhibitor screening kit from e.g., Cayman Chemical, Ann Arbor, Mich. (Cat. #560131). Using the Cayman chemical kit compounds 10, 19, 40, 43, 64, 72 and 97 were found to not significantly inhibit COX1 or COX2 at 100 μM. Representative compounds found to lower Aβ₄₂ levels using the previously described assay by at least 50% of DMSO control at concentrations ranging from 30-80 μM include compounds 1, 11, 21, 22, 24, 25, 26, 38, 43, 64, 67, 98, 100, and 102. Particularly preferred compounds of Formula I-Va for use in the methods and embodiments of the invention include those in Tables 2-7 below.

TABLE 2
Aβ₄₂ Lowering Compounds*

| cmpd | | | | |
|---|---|---|---|---|
| # | STRUCTURE | 1H NMR, δ | MS DATA | NAME |
| 1 | 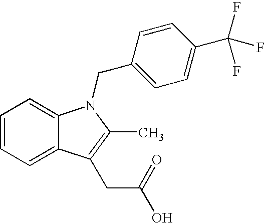 | δ 7-7.5 (8H, ArH), 5.3(2H, CH2), 2.3(3H, CH3), 3.7(2H, CH2) | neg.mode 346(M − 1), Pos.mode 348(M + 1) | 1-(4- trifluoromethylbenzyl)- 2-methylindole-3- acetic acid |
| 2 | 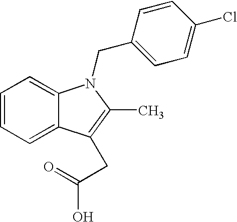 | δ 6.8-7.5 (8H, ArH), 5.26(2H, CH2), 3.7(2H, CH2), 2.3(3H, CH3) | neg.mode 312(M − 1), pos.mode 314(M + 1) | 1-(4-chlorobenzyl)-2- methylindole-3-acetic acid |
| 3 | 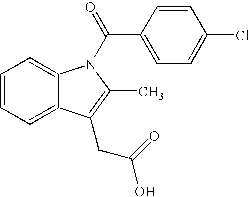 | δ 7.6-6.9 (8H, ArH), 3.7(2H, CH2), 2.39(3H, CH3) | neg.mode 325.99(M − 1) | 1-(4-chlorobenzoyl)-2- methylindole-3-acetic acid |
| 4 | 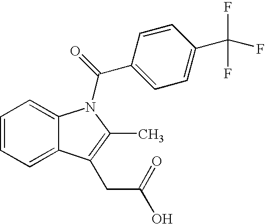 | δ 6.9-7.8 (8H, ArH), 3.7(2H, CH2), 2.3(3H, CH3) | neg.mode 360(M − 1) | 1-(4- trifluoromethylbenzoyl)- 2-methylindole-3- acetic acid |
| 5 | 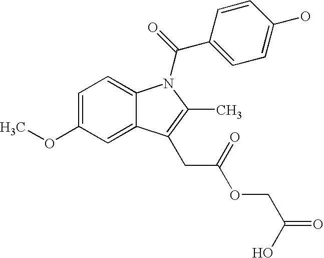 | Commercially available | | |
| 6 | 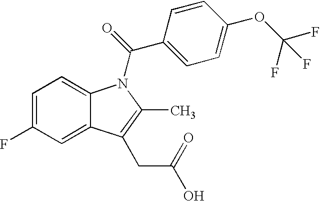 | δ 6.8-7.8 (7H, ArH); 3.7(2H, CH2); 2.39(3H, CH3); | neg.mode 393.95(M − 1) | [5-fluoro-2- methyl-1-(4- trifluoromethoxybenzoyl)- 1H-indol-3-yl] acetic acid |
| 7 | 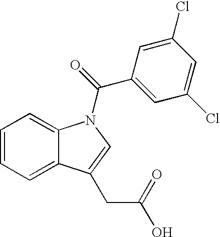 | δ 8.3(1H), 7.5-7.2(7H, ArH), 3.7(2H, CH2) | neg.mode 347(M − 1) | 1-(3,5- dichlorobenzoyl)-3- indoleacetic acid |
| 8 | 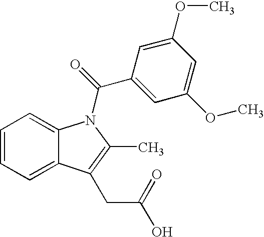 | δ 7.2-6.8 (7H, ArH), 3.8(6H, 2CH3), 3.7(2H, CH2), 2.4(3H, CH3) | neg.mode 352(M − 1) | 1-(3,5- dimethoxybenzoyl)-2- methylindole-3-acetic acid |
| 9 | 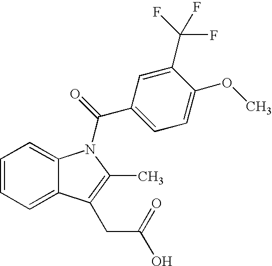 | δ 6.9-8.0 (7H, ArH), 4.03(3H, CH3), 3.6(2H, CH2), 2.39(3H, CH3) | neg.mode 390(M − 1) | 1-(4-methoxy-3- trifluoromethylbenzoyl)- 2-methylindole-3- acetic acid |
| 10 | 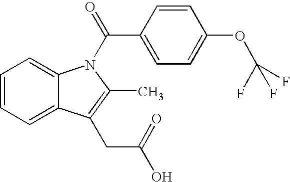 | δ 7.8-6.9 (8H, ArH), 3.7(2H, CH2), 2.4(3H, CH3) | neg.mode 377(M − 1) | 1-(4- trifluoromethoxybenzoyl)- 2-methylindole-3- acetic acid |
| 11 | 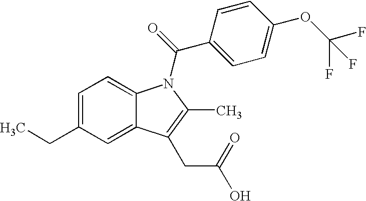 | δ 6.8-7.8 (7H, ArH); 3.7(2H, CH2); 2.7(2H, CH2); 2.4(3H, CH3); 2.21(3H, CH3) | neg.mode 404.20(M − 1) | [5-ethyl-2- methyl-1-(4- trifluoromethoxybenzoyl)- 1H-indol-3-yl] acetic acid |
| 12 | 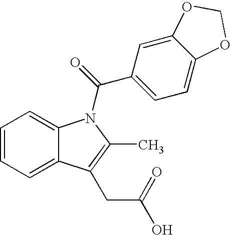 | δ 6.8-7.5 (7H, ArH), 6.08(2H, CH2), 3.7(2H, CH2), 2.4(3H, CH3) | neg.mode 336(M − 1), pos.mode 382(M + 2Na) | 1- (diperonyloylbenzoyl)- 2-methylindole-3- acetic acid |
| 13 | 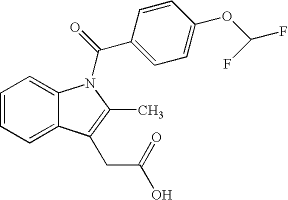 | δ 6.6-7.7 (9H, ArH), 3.7(2H, CH2), 2.4(3H, CH3) | neg.mode 358(M − 1), pos.mode 404(M + 1) | 1-(4- difluoromethoxybenzoyl)- 2-methylindole-3- acetic acid |
| 14 | 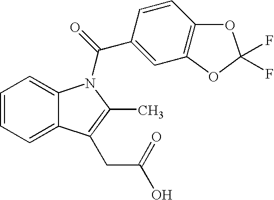 | δ 6.9-7.5 (7H, ArH), 3.7(2H, CH2), 2.4(3H, CH3) | neg.mode 372(M − 1) | 1-(2,2-difluoro-3,4- benzodioxolebenzoyl)- 2-methylindole-3- acetic acid |
| 15 | 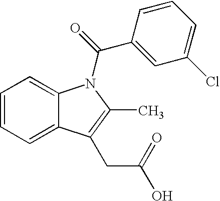 | δ 6.9-7.7 (8H, ArH), 3.7(2H, CH2), 2.4(3H, CH3) | neg.mode 327(M − 1) | 1-(5-chlorobenzoyl)-2- methylindole-3- acetic acid |
| 16 | 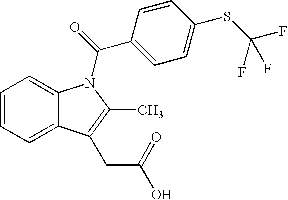 | δ 6.9-7.7 (8H, ArH), 3.7(2H, CH2), 2.39(3H, CH3) | neg.mode 326(M − 1) | 1-(4- (trifluoromethylthio) benzoyl)-2- methylindole- 3-acetic acid |
| 17 | 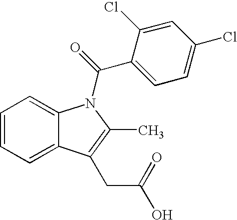 | δ 7.1-7.5 (7H, ArH), 3.7(2H, CH2), 2.28(3H, CH3) | neg.mode 361(M − 1), pos.mode 408(M + 1) | 1-(2,4- dichlorobenzoyl)-2- methylindole-3- acetic acid |
| 18 | 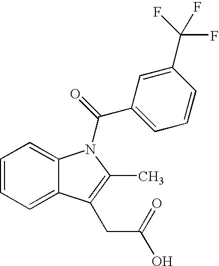 | δ 6.9-8 (8H, ArH), 3.7(2H, CH2), 2.4(3H, CH3) | neg.mode 360(M − 1), pos.mode 362(M + 1) | 1-(3- trifluoromethylbenzyl)- 2-methylindole-3- acetic acid |
| 19 | 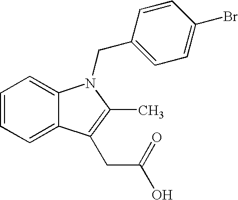 | (DMSO), δ 7.9-6.9 (8H, ArH), 5.4(2H, CH2), 3.62(2H, CH2), 2.3(3H, CH3) | neg.mode 358(M − 1), pos.mode 359(M + 1) | 1-(4-bromobenzyl)-2- methylindole-3- acetic acid |
| 20 | 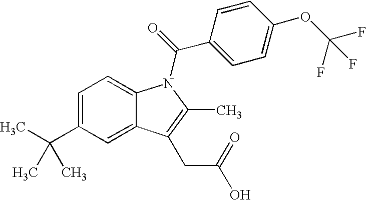 | δ 6.8-7.8 (7H, ArH), 3.7(2H, CH2), 2.4(3H, CH3), 1.4(9H, 3CH3) | neg.mode 432(M − 1) | 1-(4- trifluoromethoxy) benzoyl-5-tertbutyl-2- methylindole-3- acetic acid |
| 21 | 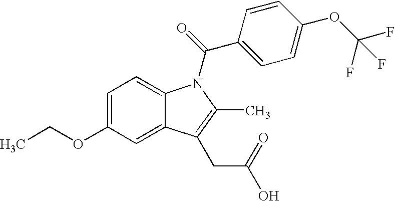 | δ 6.8-7.8 (7H, ArH); 4.1(2H, CH2); 3.7(2H, CH2); 2.39(3H, CH3); 1.41(3H, CH3) | | [5-ethoxy-2- methyl-1-(4- trifluoromethoxybenzoyl)- 1H-indol-3-yl] acetic acid |
| 22 | 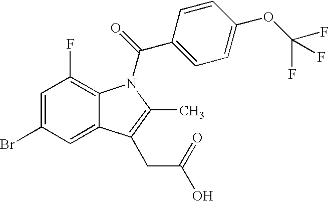 | δ 6.8-8.15 (6H, ArH); 3.7(2H, CH2); 2.41(3H, CH3); | neg.mode 473(M − 1) | [5-bromo-7-fluoro-2- methyl-1-(4- trifluoromethoxybenzoyl)- 1H-indol-3-yl] acetic acid |
| 23 | 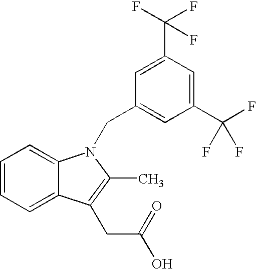 | δ 7.1-7.8 (7H, ArH); 5.41(2H, CH2); 3.8(2H, CH2); 2.38(3H, CH3). | pos.mode 416(M + 1), neg.mode 414(M − 1) | [1-(3,5- bistrifluoromethylbenzyl)- 2-methyl-1H-indol-3-yl] acetic acid |
| 24 | 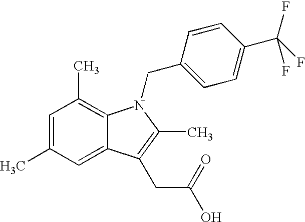 | δ 6.7-7.5 (6H, ArH); 5.59(2H, CH2); 3.78(2H, CH2); 2.42(3H, CH3); 2.4(3H, CH3), 2.22(3H, CH3). | pos.mode 376(M + 1), neg.mode 374(M − 1) | [2,5,7-trimethyl-1-(4- trifluoromethoxybenzyl)- 1H-indol-3-yl] acetic acid |
| 25 | 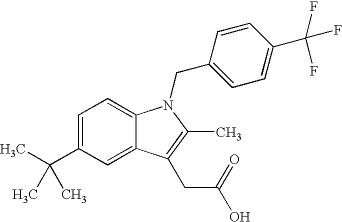 | δ 7.1-7.75 (7H, ArH); 5.5(2H, CH2); 3.61(2H, CH2); 2.22(3H, CH3); 1.35(9H, 3CH3). | pos.mode 404(M + 1), neg.mode 402(M − 1) | [5-tertbutyl-2- methyl-1-(4- trifluoromethoxybenzyl)- 1H-indol-3-yl] acetic acid |
| 26 | 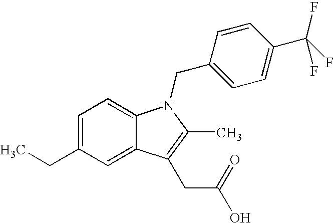 | δ 6.9-7.5 (7H, ArH); 5.5(2H, CH2); 3.61(2H, CH2); 2.62(2H, CH2); 2.22(3H, CH3), 1.2(3H, CH3). | neg.mode 374(M − 1) | [5-ethyl-2-methyl-1-(4- trifluoromethoxybenzyl)- 1H-indol-3-yl] acetic acid |
| 27 | 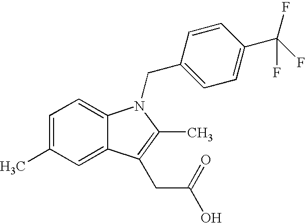 | δ 6.9-7.5 (7H, ArH); 5.3(2H, CH2); 3.7(2H, CH2); 2.45(3H, CH3); 2.25(3H, CH3), CH3). | pos.mode 362(M + 1), neg.mode 360(M − 1) | [2,5-dimethyl-1-(4- trifluoromethoxybenzyl)- 1H-indol-3-yl] acetic acid |

*The NMR data is reported for major peaks identified in the spectra, and as the skilled artisan recognizes, the NMR data is consistent with the indicated compounds.

TABLE 3
Aβ₄₂ Lowering Compounds*

| CMPD | | | MS | |
|---|---|---|---|---|
| # | STRUCTURE | 1H NMR DATA (δ, ppm) | DATA | NAME |
| 28 | 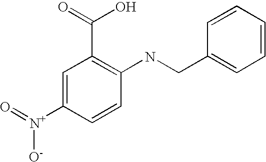 | 8.8 (d, 1H); 8.1 (dd, 1H); 7.1-7.3 (m, 5H); 6.6 (d, 1H); 4.5 (s, 2H, CH2). | 271 (M − H) | 2- benzylamino- 5-nitrobenzoic acid |
| 29 | 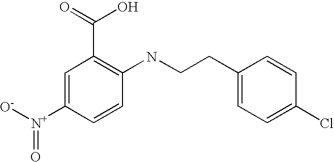 | 8.9 (d, 1H); 8.2 (dd, 1H); 7.2-7.3 (m, 4H); 6.7 (d, 1H); 3.5 (t, 2H, CH2); 3.0 (t, 2H, CH2). | 319 (M − H) | 2-[2-(4- chlorophenyl)- ethylamino]- 5-nitrobenzoic acid |
| 30 | 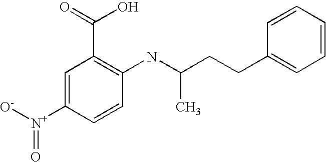 | 8.9 (d, 1H); 8.4 (d, 1NH); 8.2 (dd, 1H); 7.1-7.3 (m, 5H); 6.5 (d, 1H); 3.6 (m, 1H, CH); 2.7 (t, 2H, CH2); 2.0 (m, 2H, CH2); 1.3 (d, 3H, CH3). | 313 (M − H) | 2-(1-methyl-3- phenylpropylamino)- 5-nitrobenzoic acid |
| 31 | 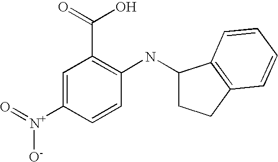 | 8.9 (d, 1H); 8.2 (dd, 1H); 7.2-7.3 (m, 4H); 6.9 (d, 1H); 5.1 (t, 1H, CH); 2.9- 3.1 (m, 2H, CH2); 2.6-2.7 (m, 1H, CH2); 1.9-2.1 (m, 1H, CH2). | 297 (M − H) | 2-(indan-1- ylamino)-5-nitrobenzoic acid |
| 32 | 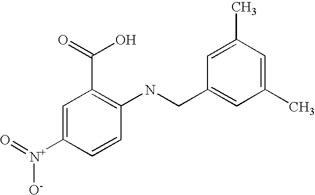 | 8.9 (d, 1H); 8.1 (dd, 1H); 7.4 (s, 1H); 6.9 (s, 2H); 6.7 (d, 1H); 4.5 (s, 2H, CH2); 2.3 (s, 6H, 2 × CH3). | 299 (M − H) | 2-(3,5- dimethylbenzylamino)- 5-nitrobenzoic acid |
| 33 | 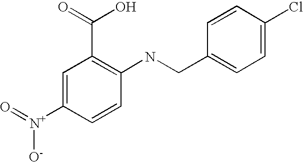 | 9.0 (d, 1H); 8.2 (dd, 1H); 7.3-7.4 (m, 4H); 6.6 (d, 1H); 4.6 (s, 2H, CH2). | 306 (M − H) | 2-(4- chlorobenzylamino)- 5-nitrobenzoic acid |
| 34 | 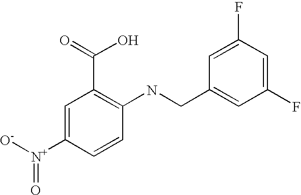 | 8.8 (d, 1H); 8.1 (dd, 1H); 6.8 (d, 2H); 6.7 (t, 1H); 6.5 (d, 1H); 4.5 (s, 2H, CH2). | 307 (M − H) | 2-(3,5- difluorobenzylamino)- 5-nitrobenzoic acid |
| 35 | 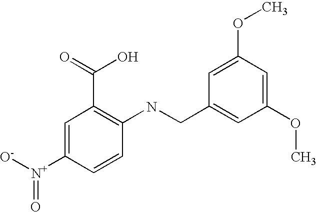 | 8.9 (d, 1H); 8.1 (dd, 1H); 6.6 (d, 1H); 6.4 (s, 2H); 6.3 (s, 1H); 4.4 (s, 2H, CH2); 3.7 (s, 6H, 2 × OCH3). | 331 (M − H) | 2-(3,5- dimethoxybenzylamino)- 5-nitrobenzoic acid |
| 36 | 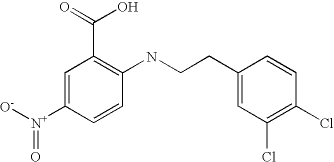 | 8.9 (m, 1H); 8.2 (m, 1H); 7.2-7.4 (m, 2H); 7.1 (m, 1H); 6.7 (m, 1H); 3.5 (m, 1H, CH2); 3.0 (m, 1H, CH2). | 3553/355 (M − H) | 2-[2-(3,4- dichlorophenyl)- ethylamino]- 5-nitrobenzoic acid |
| 37 | 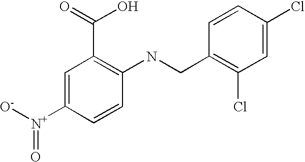 | 8.9 (d, 1H); 8.1 (dd, 1H); 7.4 (s, 1H); 7.2 (s, 2H); 6.5 (d, 1H); 4.6 (s, 2H, CH2). | 339/341 (M − H) | 2-(2,4- dichlorobenzylamino)- 5-nitrobenzoic acid |
| 38 | 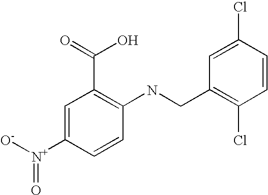 | 8.9 (d, 1H); 8.1 (dd, 1H); 7.2-7.3 (m, 3H); 6.5 (d, 1H); 4.6 (s, 2H, CH2). | 339/341 (M − H) | 2-(2,5- dichlorobenzylamino)- 5-nitrobenzoic acid |
| 39 | 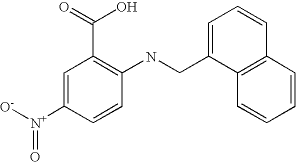 | 8.9 (d, 1H); 8.2 (dd, 1H); 7.8-7.9 (m, 2H); 7.7 (m, 1H); 7.5 (m, 2H); 7.4 (m, 2H); 6.7 (d, 1H); 4.9 (s, 2H, CH2). | 321 (M − H) | 2- [(naphthalen- 1-ylmethyl)- amino]-5-nitrobenzoic acid |
| 40 | 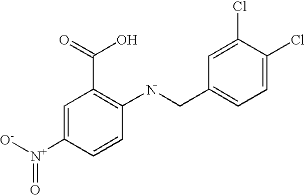 | 8.9 (d, 1H); 8.1 (dd, 1H); 7.4 (m, 2H); 7.1 (m, 1H); 6.5 (d, 1H); 4.4 (s, 2H, CH2). | | 2-(3,4- dichlorobenzylamino)- 5-nitrobenzoic acid |
| 41 | 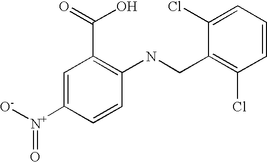 | 8.8 (d, 1H); 8.2 (dd, 1H); 7.2-7.3 (m, 3H); 6.9 (d, 1H); 4.8 (s, 2H, CH2). | 339/341 (M − H) | 2-(2,6- dichlorobenzylamino)- 5-nitrobenzoic acid |
| 42 | 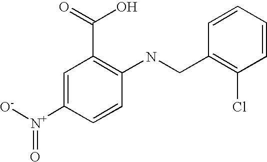 | 8.9 (d, 1H); 8.1 (dd, 1H); 7.3 (m, 1H); 7.1-7.2 (m, 3H); 6.5 (d, 1H); 4.6 (s, 2H, CH2). | 305 (M − H) | 2-(2- chlorobenzylamino)- 5-nitrobenzoic acid |
| 43 | 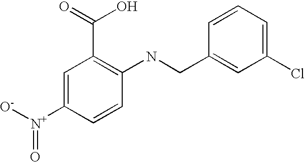 | 8.9 (d, 1H); 8.1 (dd, 1H); 7.1-7.3 (m, 4H); 6.5 (d, 1H); 4.5 (s, 2H, CH2). | 305 (M − H) | 2-(3- chlorobenzylamino)- 5-nitrobenzoic acid |
| 44 | 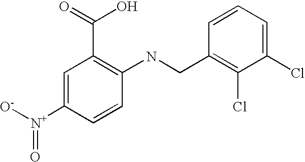 | 8.9 (m, 1H); 8.2 (m, 1H); 7.1-7.5 (m, 3H); 6.5 (m, 1H); 4.6 (s, 2H, CH2). | 339/341 (M − H) | 2-(2,3- dichlorobenzylamino)- 5-nitrobenzoic acid |
| 45 | 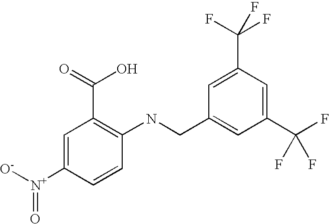 | 9.0 (m, 1H); 8.2 (m, 1H); 7.8 (m, 3H); 6.6 (m, 1H); 4.7 (m, 2H, CH2). | 407 (M − H) | 2-(3,5-bis- trifluoromethyl benzylamino)- 5-nitrobenzoic acid |
| 46 | 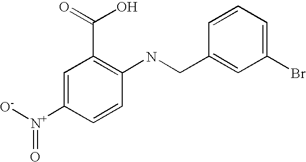 | 8.9 (d, 1H); 8.1 (dd, 1H); 7.4 (m, 2H); 7.1-7.3 (m, 2H); 6.5 (d, 1H); 4.5 (s, 2H, CH2). | 350 (M − H) | 2-(3- bromobenzylamino)- 5-nitrobenzoic acid |
| 47 | 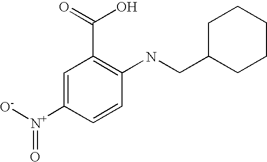 | 8.9 (d, 1H); 8.5 (br. m, 1H); 8.2 (dd, 1H); 6.7 (d, 1H); 3.2 (t, 2H, CH2); 1.7-1.9 (m, 6H); 1.0-1.3 (m, 5H). | 277 (M − H) | 2- (cyclohexylmethylamino)- 5-nitrobenzoic acid |
| 48 | 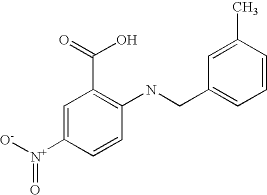 | 8.9 (d, 1H); 8.1 (dd, 1H); 7.0-7.2 (m, 4H); 6.6 (d, 1H); 4.5 (s, 2H, CH2). | 285 (M − H) | 2-(3- methylbenzylamino)- 5-nitrobenzoic acid |
| 49 | 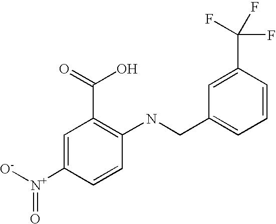 | 8.9 (d, 1H); 8.1 (dd, 1H); 7.5-7.6 (m, 4H); 6.6 (d, 1H); 4.6 (s, 2H, CH2). | 339 (M − H) | 2-(3- trifluoromethyl benzylamino)- 5-nitrobenzoic acid |
| 50 | 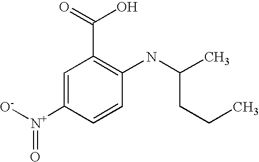 | 8.9 (d, 1H); 8.4 (d, 1H); 8.2 (dd, 1H); 6.7 (d, 1H); 3.7 (m, 1H, CH); 1.3-1.7 (m, 7H); 1.0 (t, 3H, CH3). | 251 (M − H) | 2-(1-methyl- butylamino)- 5-nitrobenzoic acid |
| 51 | 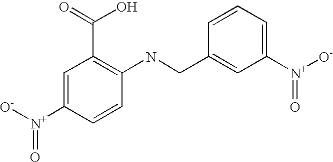 | 8.9 (d, 1H); 8.1 (m, 3H); 7.5-7.6 (m, 2H); 6.5 (d, 1H); 4.6 (s, 2H, CH2). | 316 (M − H) | 2-(3- nitrobenzylamino)- 5-nitrobenzoic acid |
| 52 | 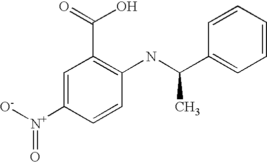 | 8.8 (d, 1H); 7.9 (dd, 1H); 7.1-7.3 (m, 5H); 6.4 (d, 1H); 4.6 (m, 1H, CH); 1.5 (d, 3H, CH3). | 285 (M − H) | 2-[(R)-1- phenylethylamino)- 5-nitrobenzoic acid |
| 53 | 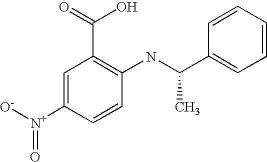 | 8.9 (d, 1H); 8.0 (dd, 1H); 7.2-7.3 (m, 5H); 6.4 (d, 1H); 4.6 (m, 1H, CH); 1.6 (d, 3H, CH3). | 285 (M − H) | 2-[(S)-1- phenylethylamino)- 5-nitrobenzoic acid |
| 54 | 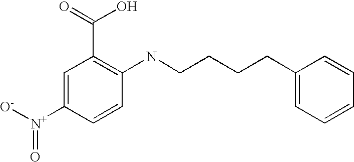 | 8.8 (d, 1H); 8.1 (dd, 1H); 7.1-7.3 (m, 5H); 6.6 (d, 1H); 3.3 (t, 2H, CH2); 2.6 (t, 2H, CH2); 1.7 (m, 4H, 2 × CH2). | 313 (M − H) | 5-nitro-2-(4- phenylbutylamino) benzoic acid |
| 55 | 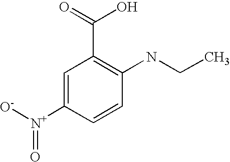 | 8.8 (d, 1H); 8.2 (dd, 1H); 6.6 (d, 1H); 3.2 (m, 2H, CH2); 1.3 (t, 3H, CH3). | 209 (M − H) | 2-ethylamino- 5-nitrobenzoic acid |
| 56 | 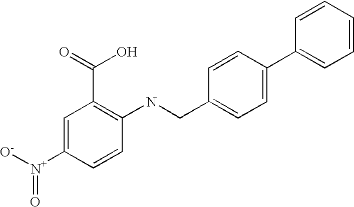 | 8.9 (d, 1H); 8.1 (dd, 1H); 7.5-7.6 (m, 4H); 7.3-7.4 (m, 5H); 6.5 (d, 1H); 4.5 (s, 2H, CH2). | 347 (M − H) | 2-[(biphenyl- 4-ylmethyl)- amino]-5- nitrobenzoic acid |

*The skilled artisan understands that the compounds in table 2 that have an —N— group have the valences completed with a hydrogen; that is they are —NH— groups; the negative mode MS data reported; the NMR data is reported for major peaks identified in the spectra, and as the skilled artisan recognizes, the NMR data is consistent with the indicated compounds.

TABLE 4
Aβ₄₂ Lowering Compounds*

| CMPD | | | MS | |
|---|---|---|---|---|
| # | STRUCTURE | 1H NMR DATA | DATA | NAME |
| 57 | 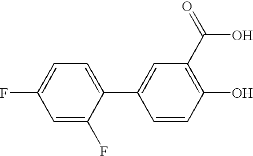 | Commercially Available | | 2′,4′-Difluoro-4- hydroxy-biphenyl-3- carboxylic acid |
| 58 | 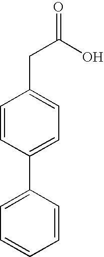 | Commercially Available | | Biphenyl-4-yl-acetic acid |
| 59 | 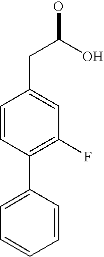 | δ 7.6-7.5 (m, 2H), 7.5- 7.3 (m, 4H), 7.2-7.1 (m, 2H), 3.70 (s, 2H) | 229 (M − 1) 230 (M + 1) | (2-Fluoro-biphenyl- 4-yl)-acetic acid |
| 60 | 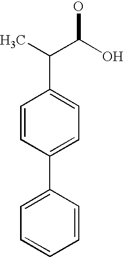 | δ 7.6-7.5 (m, 4H), 7.5- 7.3 (m, 5H), 3.80 (q, J = 7.2Hz, 1H), 1.56 (d, J = 7.2Hz, 3H) | 225 (M − 1) | 2-Biphenyl-4-yl- propionic acid |
| 61 | 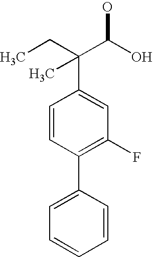 | δ 7.6-7.1 (m, 8H), 2.12 (m, 1H), 2.03 (m, 1H), 1.60 (s, 3H), 0.90 (app t, J = 7.4 Hz, 3H) | 272 (M − 1) | 2-(2-Fluoro- biphenyl-4-yl)-2- methyl-butyric acid |
| 62 | 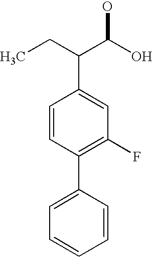 | δ 7.6-7.5 (m, 2H), 7.5- 7.3 (m, 4H), 7.2-7.1 (m, 2H), 3.51 (app t, J = 7.7 Hz, 1H), 2.12 (m, 1H), 1.88 (m, 1H), 0.96 (app t, J = 7.4 Hz, 3H) | 258 ([M + 1]); 214 (M − CO₂H) | 2-(2-Fluoro- biphenyl-4-yl)- butyric acid |
| 63 | 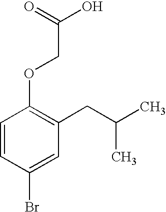 | δ 7.3-7.2 (m, 2H), 6.61 (m, 1H), 4.66 (s, 2H), 2.50 (d, J = 7.1 Hz, 2H), 1.92 (m, 1H), 0.91 (d, J = 6.6 Hz, 6H) | 287 (M − 1) | (4-Bromo-2- isobutyl-phenoxy)- acetic acid |
| 64 | 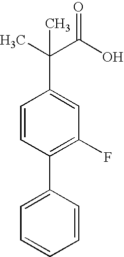 | (400 MHz) δ 7.56- 7.51 (m, 2H), 7.47-7.34 (m, 4H), 7.28-7.19 (m, 2H), 1.64 (s, 6H) | 214 (M − CO₂H) | 2-(2-Fluoro- biphenyl-4-yl)-2- methyl-propionic acid |
| 65 | 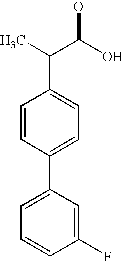 | δ 7.54 (m, 2H), 7.5-7.2 (m, 5H), 7.03 (m, 1H), 3.80 (q, J = 7.1 Hz, 1H), 1.56 (d, J = 7.2 Hz, 3H) | 200 (M − CO2H) | 2-(3′-Fluoro- biphenyl-4-yl)- propionic acid |
| 66 | 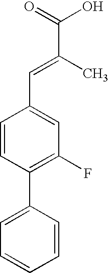 | δ 7.80 (m, 1H), 7.59 (m, 2H), 7.6-7.2 (m, 6H), 2.21 (d, J = 1.3 Hz, 3H) | 256 (M + 1) 255 (M − 1) | (E)-3-(2-Fluoro- biphenyl-4-yl)-2- methyl-acrylic acid |
| 67 | 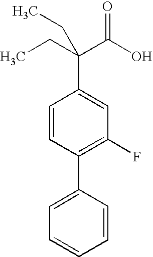 | δ 7.6-7.5 (m, 2H), 7.5- 7.3 (m, 4H), 7.2-7.0 (m, 2H), 2.10 (m, 4H), 0.82 (app t, J = 7.4 Hz, 6H) | 286 (M + 1) 285 (M − 1) | 2-Ethyl-2-(2-fluoro- biphenyl-4-yl)- butyric acid |

*The NMR data is reported for major peaks identified in the spectra, and as the skilled artisan recognizes, the NMR data is consistent with the indicated compounds.

TABLE 5
Aβ₄₂ Lowering Compounds

| CMPD | | |
|---|---|---|
| # | STRUCTURE | NAME |
| 68 | 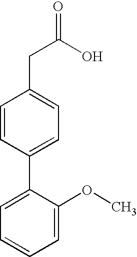 | (2′-Methoxy-biphenyl-4- yl)-acetic acid |
| 69 | 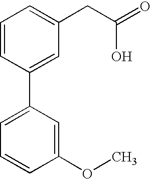 | (3′-Methoxy-biphenyl-3- yl)-acetic acid |
| 70 | 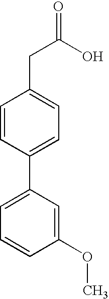 | (3′-Methoxy-biphenyl-4- yl)-acetic acid |
| 71 | 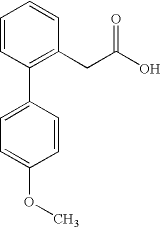 | (4′-Methoxy-biphenyl-2- yl)-acetic acid |
| 72 | 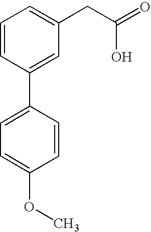 | (4′-Methoxy-biphenyl-3- yl)-acetic acid |
| 73 | 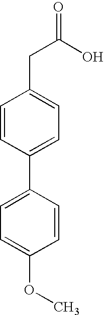 | (4′-Methoxy-biphenyl-4- yl)-acetic acid |
| 74 | 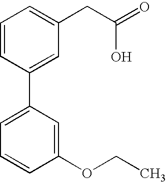 | (3′-Ethoxy-biphenyl-3-yl)- acetic acid |
| 75 | 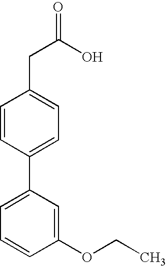 | (3′-Ethoxy-biphenyl-4-yl)- acetic acid |
| 76 | 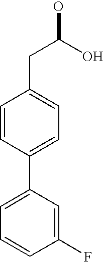 | (3′-Fluoro-biphenyl-4-yl)- acetic acid |
| 77 | 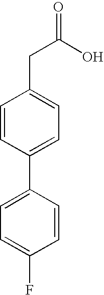 | (4′-Fluoro-biphenyl-4-yl)- acetic acid |
| 78 | 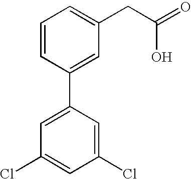 | (3′,5′-Dichloro-biphenyl-3- yl)-acetic acid |
| 79 | 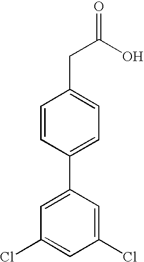 | (3′,5′-Dichloro-biphenyl-4- yl)-acetic acid |
| 80 | 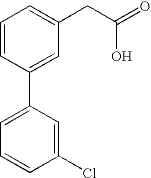 | (3′-Chloro-biphenyl-3-yl)- acetic acid |
| 81 | 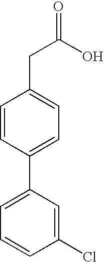 | (3′-Chloro-biphenyl-4-yl)- acetic acid |
| 82 | 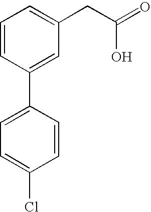 | (4′-Chloro-biphenyl-3-yl)- acetic acid |
| 83 | 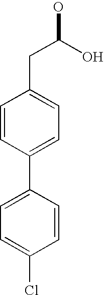 | (4′-Chloro-biphenyl-4-yl)- acetic acid |
| 84 | 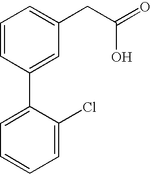 | (2′-Chloro-biphenyl-3-yl)- acetic acid |
| 85 | 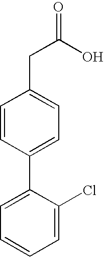 | (2′-Chloro-biphenyl-4-yl)- acetic acid |
| 86 | 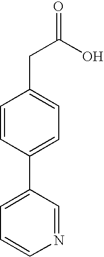 | (4-Pyridin-3-yl-phenyl)- acetic acid |
| 87 | 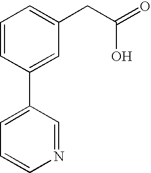 | (3-Pyridin-3-yl-phenyl)- acetic acid |
| 88 | 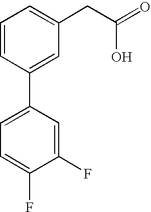 | (3′,4′-Difluoro-biphenyl-3- yl)-acetic acid |
| 89 | 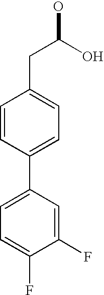 | (3′,4′-Difluoro-biphenyl-4- yl)-acetic acid |
| 90 | 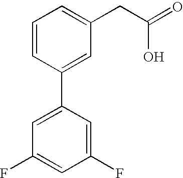 | (3′,5′-Difluoro-biphenyl-3- yl)-acetic acid |
| 91 | 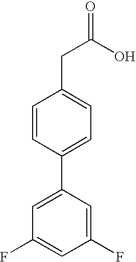 | (3′,5′-Difluoro-biphenyl-4- yl)-acetic acid |
| 92 | 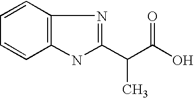 | 2-(1H-Benzoimidazol-2- yl)-propionic acid |

TABLE 6
Aβ₄₂ Lowering Compounds*

| CMPD | | | | |
|---|---|---|---|---|
| # | STRUCTURE | 1H NMR DATA | MS DATA | NAME |
| 93 | 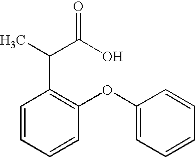 | δ 7.4-6.8 (m, 9H), 4.11 (q, J = 7.2 Hz, 1H), 1.50 (d, J = 7.2 Hz, 3H) | 241 (M − 1) | 2-(2-Phenoxy- phenyl)-propionic acid |
| 94 | 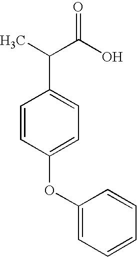 | δ 7.4-6.9 (m, 9H), 3.37 (q, J = 7.2 Hz, 1H), 1.52 (d, J = 7.2 Hz, 3H) | 241 (M − 1) | 2-(4-Phenoxy- phenyl)-propionic acid |
| 95 | 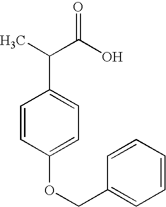 | δ 7.5-7.2 (m, 7H), 6.94 (m, 2H), 5.05 (s, 2H), 3.70 (q, J = 7.2 Hz, 1H), 1.49 (d, J = 7.2 Hz, 3H) | 255 (M − 1) | 2-(4-Benzyloxy- phenyl)-propionic acid |
| 96 | 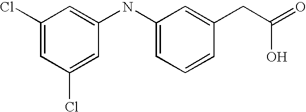 | δ 3.62 (s, 2H), 6.83-7.32 (m, 7H) | 295 (M + 1) | [3-(3,5-Dichloro- phenylamino)- phenyl]-acetic acid |

*The skilled artisan understands that the compounds in Table 5 that have an —N— group have their valences completed with a hydrogen; that is they are —NH— groups; the NMR data is reported for major peaks identified in the spectra, and as the skilled artisan recognizes, the NMR data is consistent with the indicated compounds.

TABLE 7
Aβ₄₂ Lowering Compounds*

| Compound | | | | |
|---|---|---|---|---|
| Number | Structure | 1H NMR, δ | MS | Name |
| 97 | 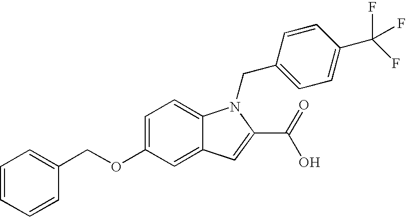 | 7.7-6.9 (12H, ArH); 6 (2H, CH2), 5.1 (2H, CH2 | neg. mode 424 (M − H) | 5-benzyloxy- 1-(4- trifluoromethylbenzyl)- 1H-indole-2- carboxylic acid |
| 98 | 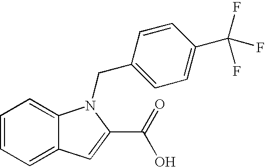 | 7.8-7.1 (9H, ArH), 5.9 (2H, CH2) | neg. mode 318.05 (M − H) | 1-(4- trifluoromethylbenzyl)- 1H-indole-2- carboxylic acid |
| 99 | 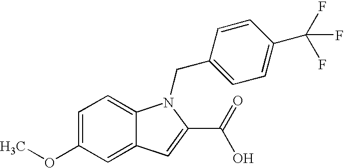 | 7.7-6.9 (8H, ArH), 5.9 (2H, CH2), 3.8 (3H, CH3) | neg. mode 348 (M − H), pos. mode 350 (M + H) | 5-methoxy-1-(4- trifluoromethylbenzyl)- 1H-indole-2- carboxylic acid |
| 100 | 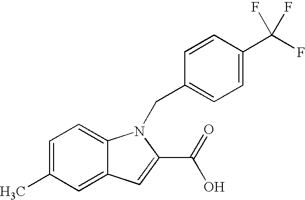 | 7.7-7.1 (8H, ArH), 5.9 (2H, CH2), 2.4 (3H, CH3) | neg. mode 332.04 (M − H), pos. mode 334 (M + H) | 5-methyl-1-(4- trifluoromethylbenzyl)- 1H-indole-2- carboxylic acid |
| 101 | 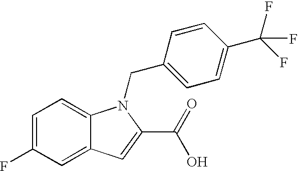 | 7.7-7.1 (8H, ArH), 5.9 (2H, CH2) | neg. mode 336.01 (M − H) | 5-fluoro-1-(4- trifluoromethylbenzyl)- 1H-indole-2- carboxylic acid |
| 102 | 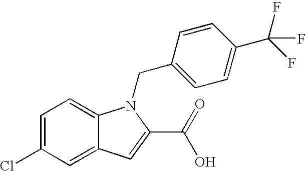 | 7.8-7.0 (8H, ArH), 5.9 (2H, CH2) | neg. mode 352 (M − H), pos. mode 399 (M + 2Na) | 5-chloro-1-(4- trifluoromethylbenzyl)- 1H-indole-2- carboxylic acid |
| 103 | 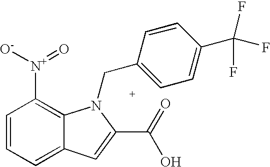 | 8.2-6.9 (8H-ArH), 6 (2H, CH2) | neg. mode 363 (M − H) | 7-nitro-1-(4- trifluoromethylbenzyl)- 1H-indole-2- carboxylic acid |
| 104 | 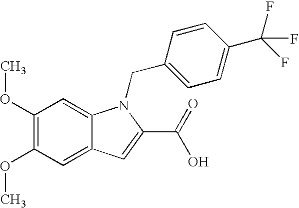 | 7.7-7.1 (7H, ArH), 5.9 (2H, CH2) | neg. mode 378 (M − H), pos. mode 380 (M + H) | 5,6-dimethoxy-1-(4- trifluoromethylbenzyl)- 1H-indole-2- carboxylic acid |
| 105 | 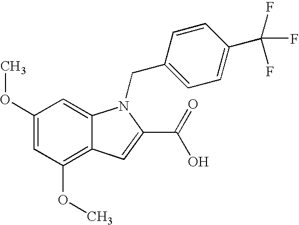 | 7.7-6.2 (7H, ArH), 5.9 (2H, CH2) | neg. mode 396.9 (M − H), pos. mode 380 (M + H) | 4,6-dimethoxy-1-(4- trifluoromethylbenzyl)- 1H-indole-2- carboxylic acid |
| 106 | 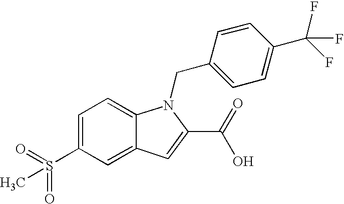 | 8.4-7.2 (8H-ArH), 5.9 (2H-CH2), 3.1 (3H-CH3) | neg. mode 396.9 (M − H), pos. mode 414 (M + H2O) | 5-methanesulfonyl- 1-(4- trifluoromethylbenzyl)- 1H-lindole-2- carboxylic acid |
| 107 | 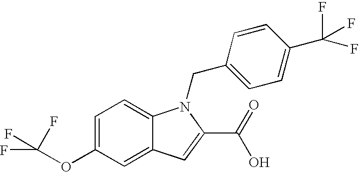 | 7.8-7.2 (8H, ArH), 6 (2H, CH2) | neg. mode 402 (M − H) | 5- trifluoromethoxy- 1-(4- trifluoromethylbenzyl)- 1H-indole-2- carboxylic acid |
| 108 | 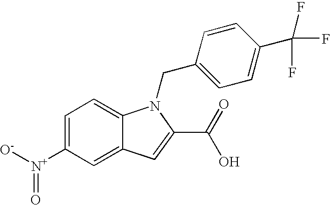 | 8.9-7.1 (8H-ArH), 6 (2H, CH2) | neg. mode 363 (M − H) | 5-nitro-1-(4- trifluoromethylbenzyl- 1H-indole-2- carboxylic acid |
| 109 | 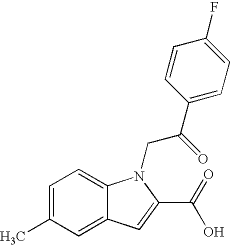 | 8.2-7.0 (8H, ArH), 6.2 (2H, CH2) | neg. mode 310 (M − H) | 1-[2-(4- fluorophenyl)- 2-oxoethyl]-5- methyl-1H- indole-2- carboxylic acid |
| 110 | 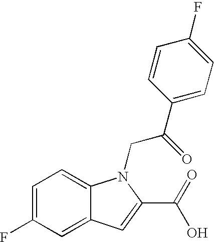 | 8.2-7.0 (8H, ArH), 6.3 (2H, CH2) | neg. mode 314 (M − H) | 5-fluoro-1-[2-(4- fluorophenyl)- 2-oxoethyl]- 1H-indole-2- carboxylic acid |
| 111 | 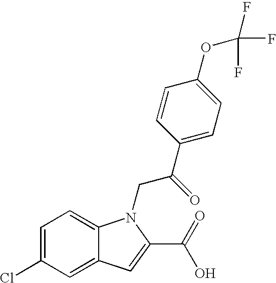 | 8.3-6.9 (8H, ArH), 6.3 (2H, CH2) | neg. mode 396 (M − H) | 5-chloro-1-[2-oxo-2-(4- trifluoromethoxyphenyl)- ethyl]-1H-indole-2- carboxylic acid |
| 112 | 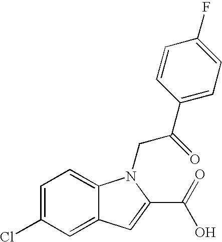 | 8.2-6.8 (8H, ArH), 6.3 (2H, CH2) | neg. mode 330 (M − H) | 5-chloro-1-[2-(4- fluorophenyl)- 2-oxoethyl]- 1H-indole-2- carboxylic acid |

*The NMR data is reported for major peaks identified in the spectra, and as the skilled artisan recognizes, the NMR data is consistent with the indicated compounds.

The present invention also encompasses the following compounds that were found to be capable of lowering Aβ₄₂ level in the assays described herein.

Compounds that were synthesized in an analogous manner to that described for compound 20 include:

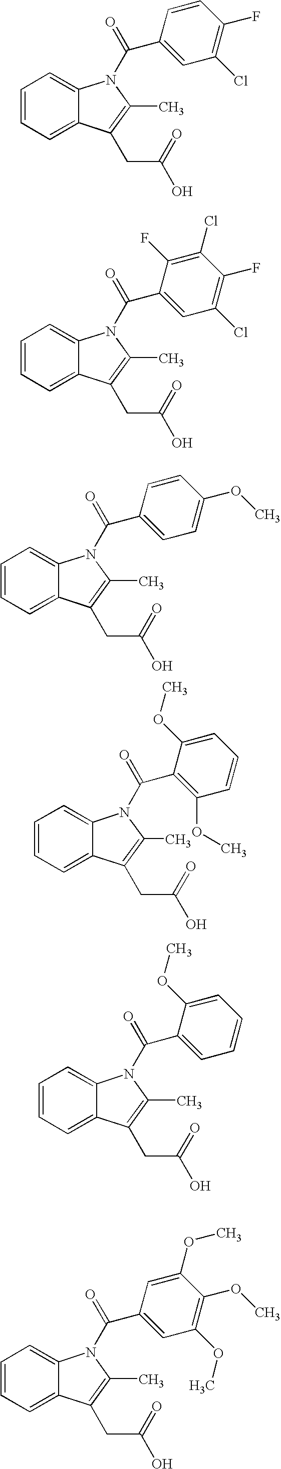

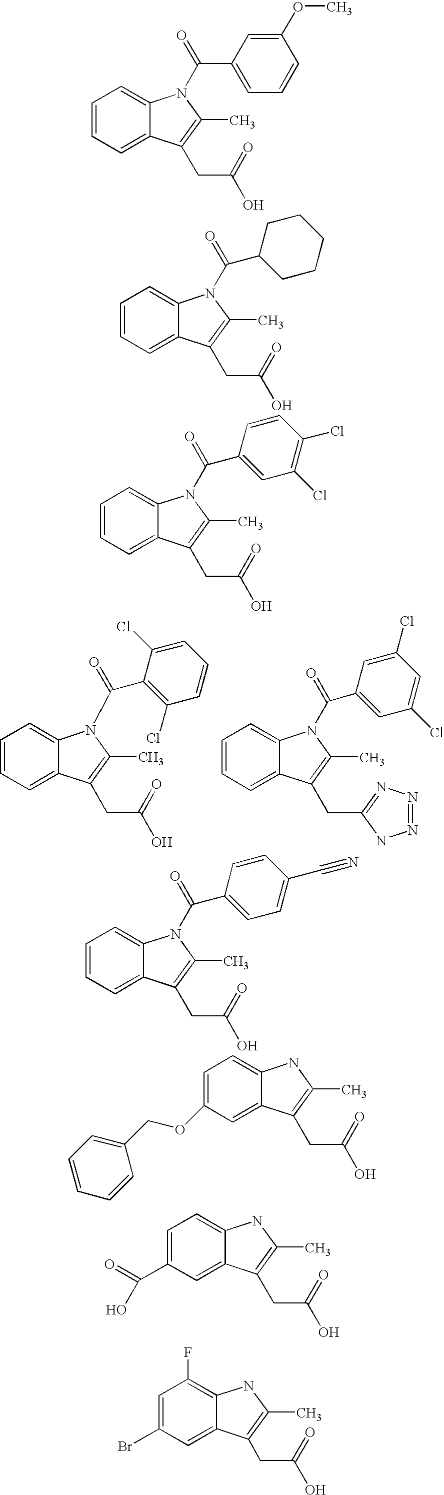

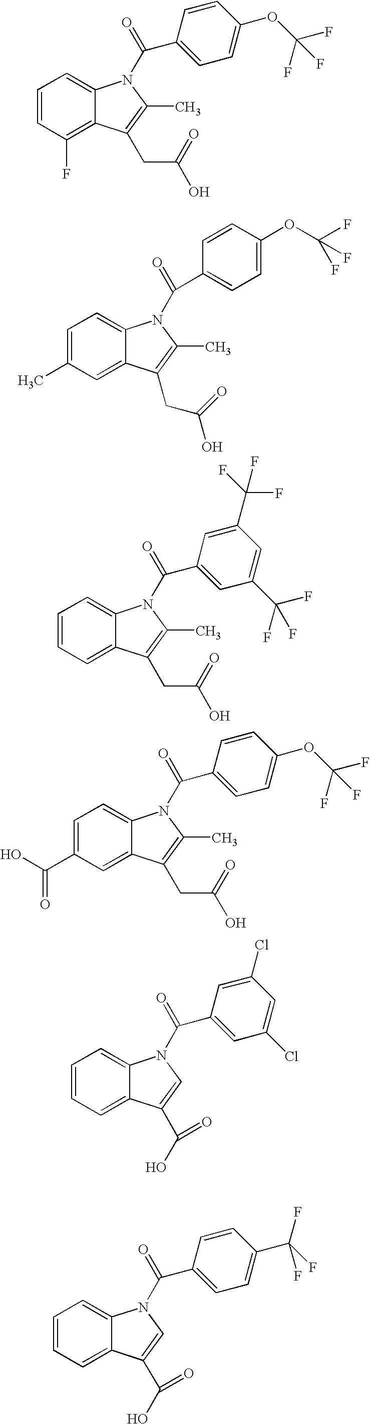

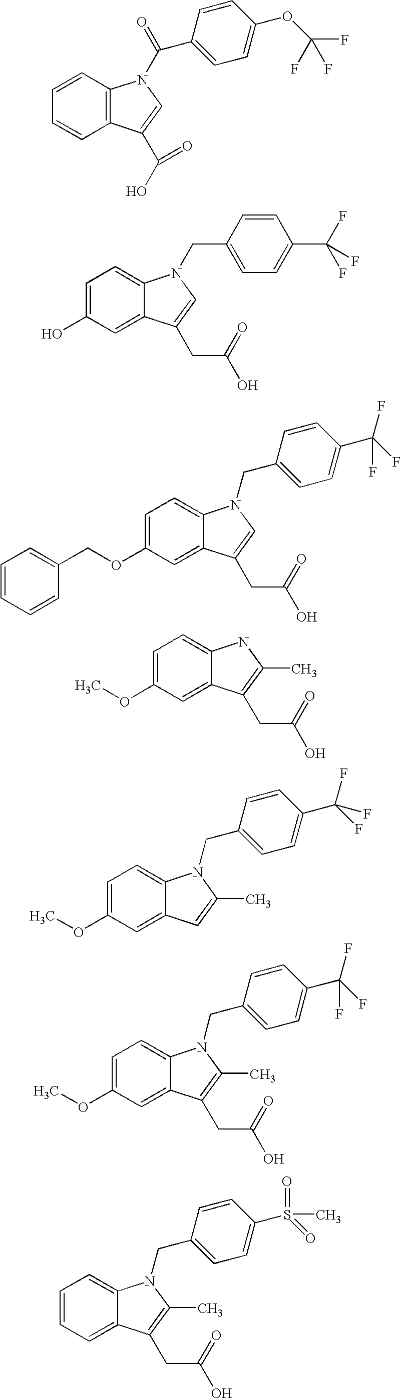

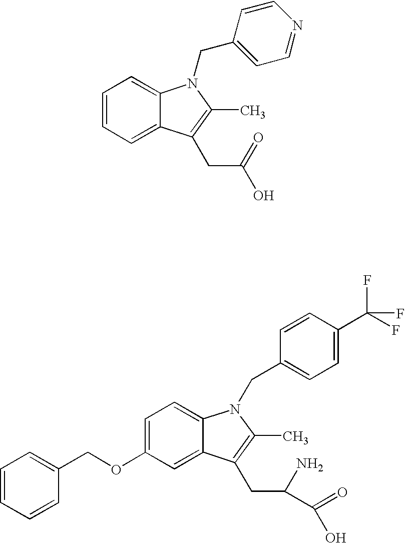

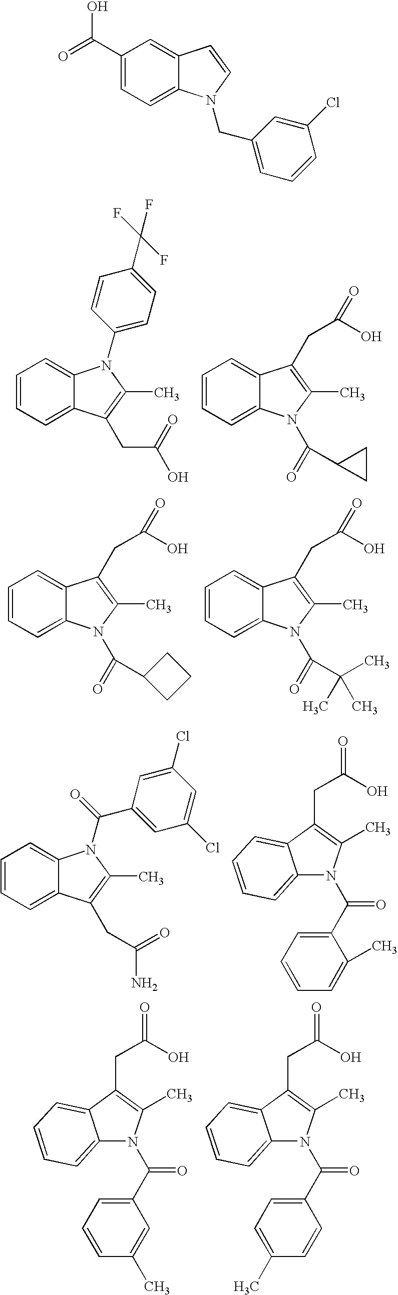

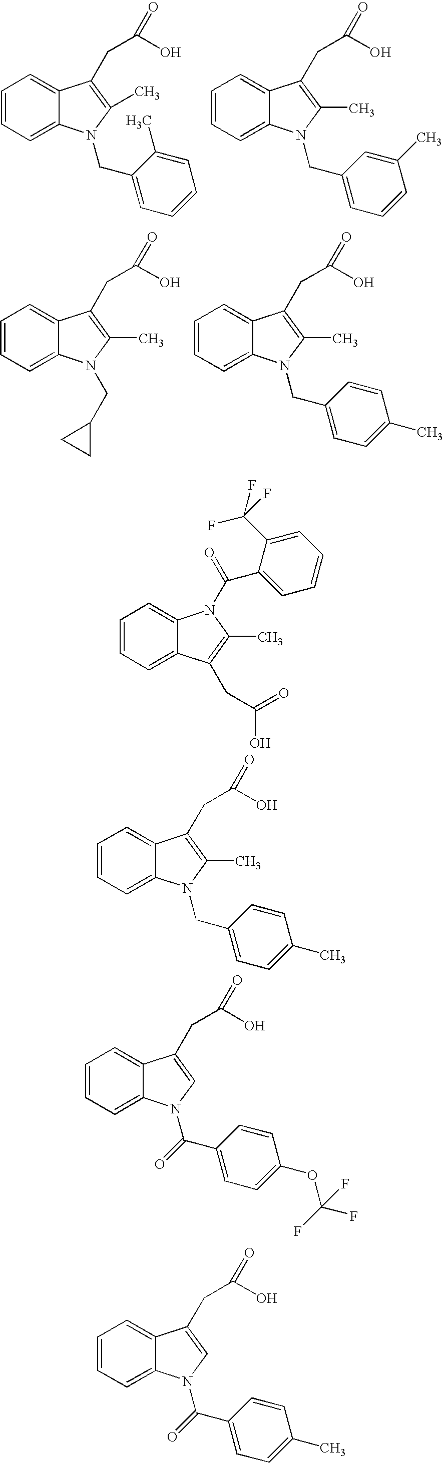

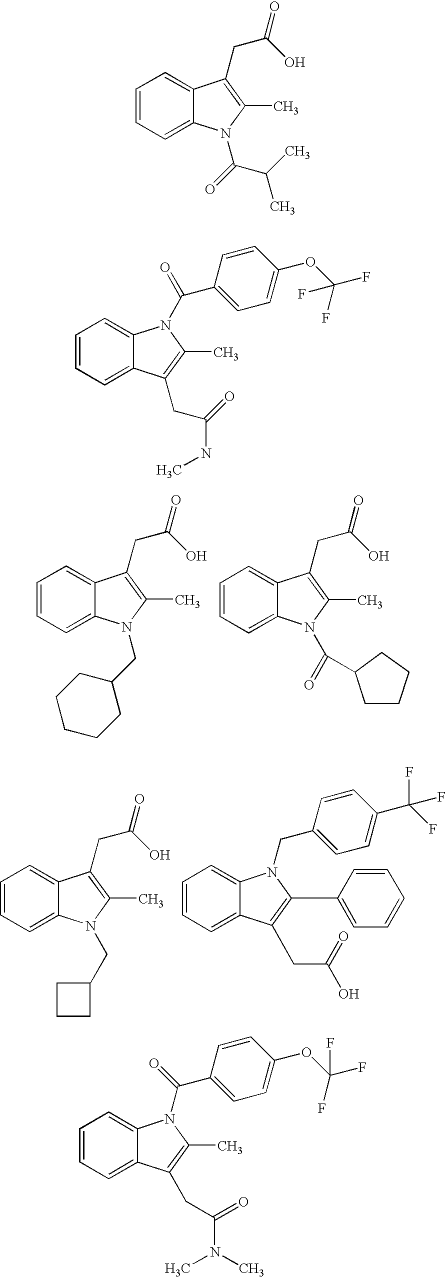

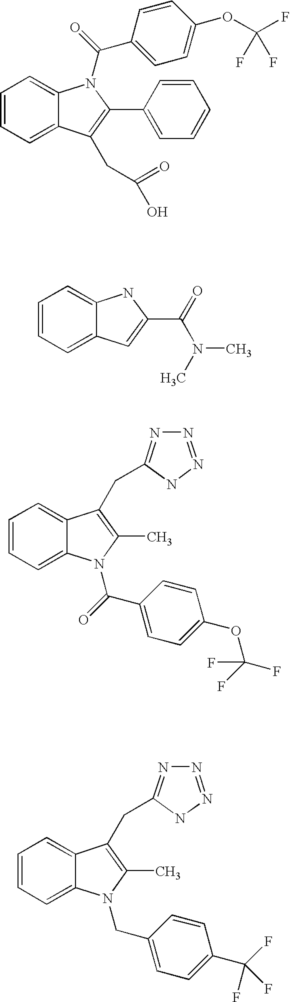

The following compounds were synthesized in an analogous manner to that described for compound 40:

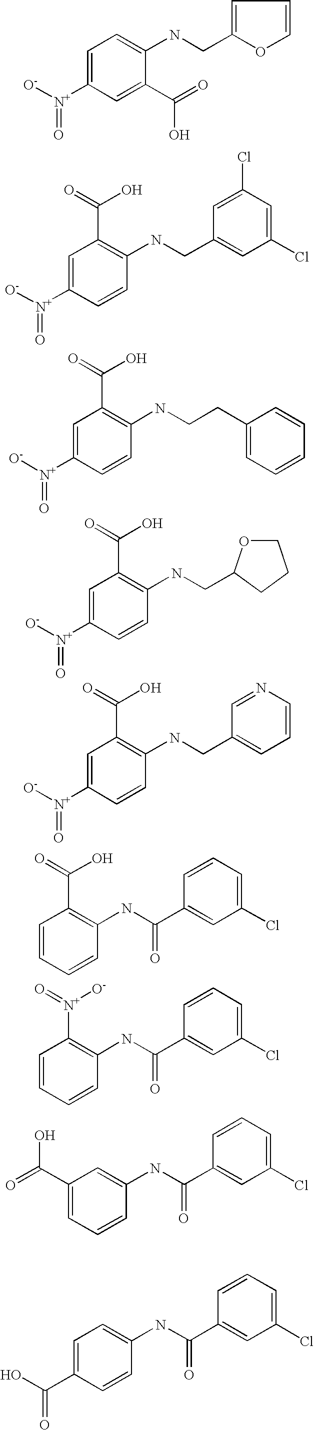

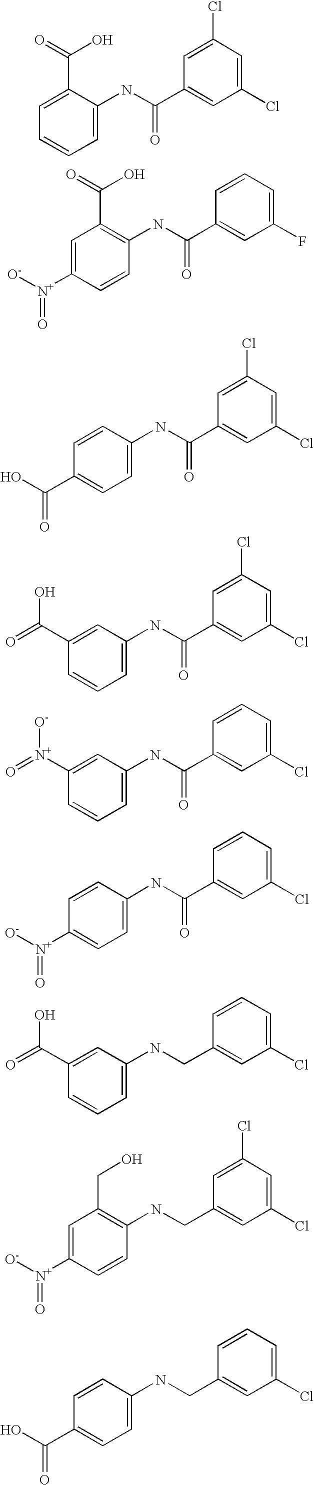

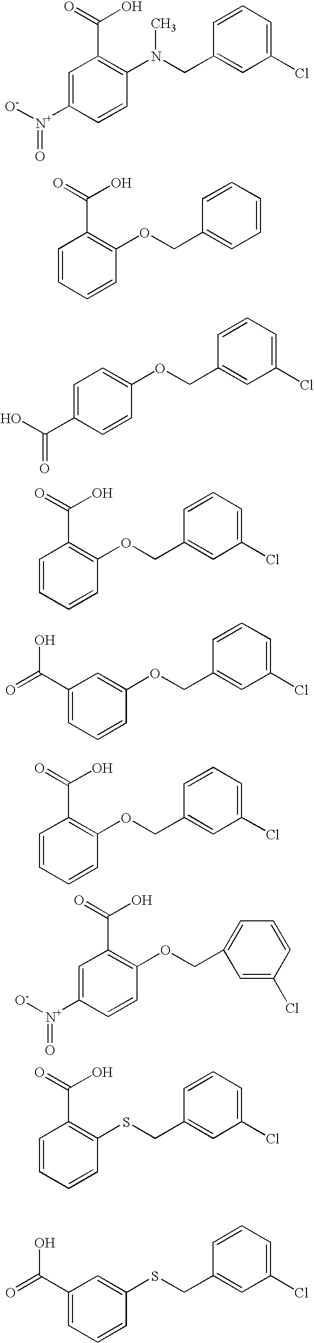

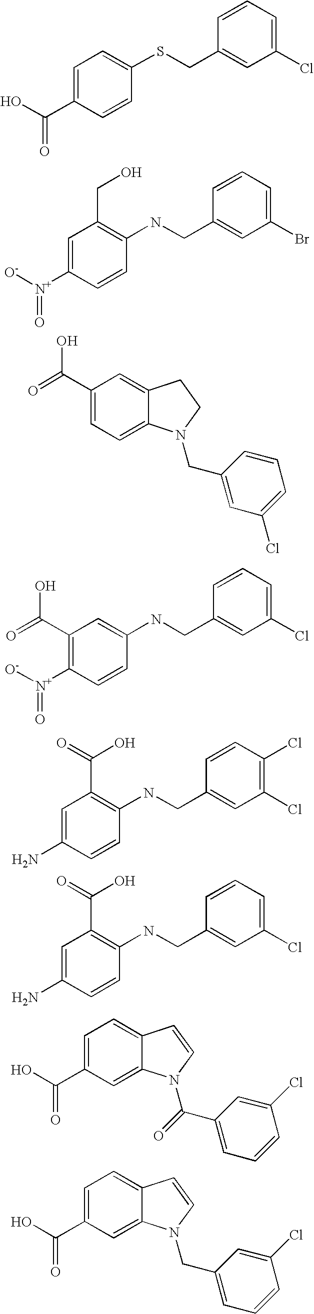

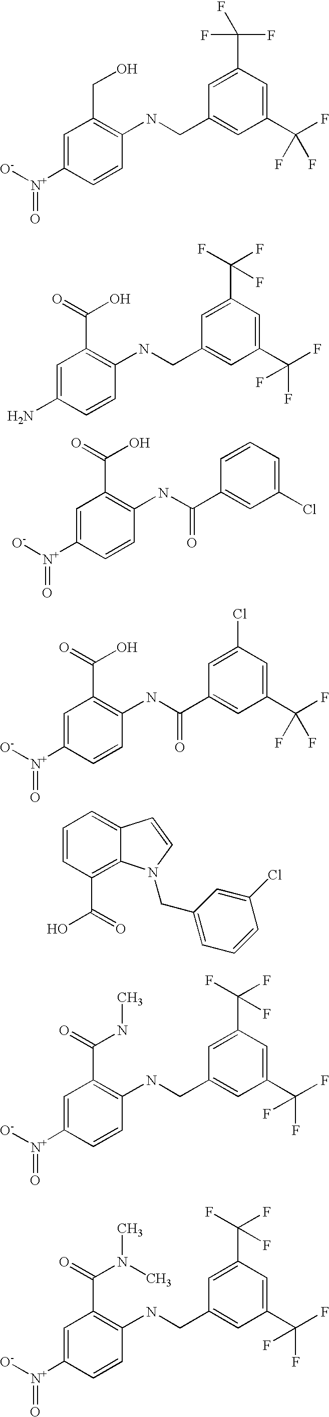

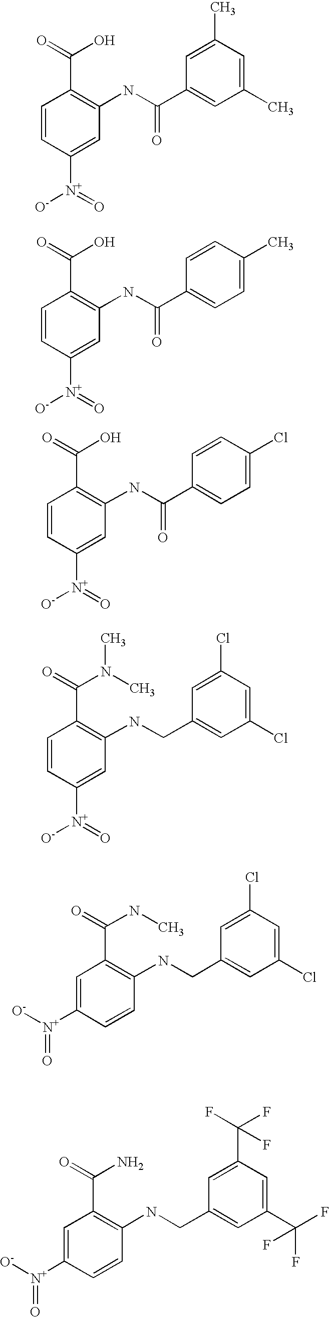

The following compounds were synthesized in an analogous manner to that described for compound 56, or commercially available:

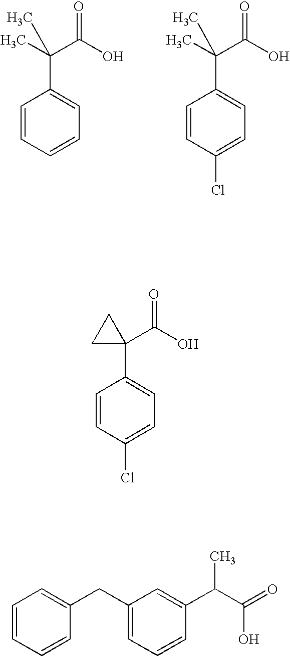

The following compounds were synthesized in an analogous manner to that described for compound 62, or commercially available:

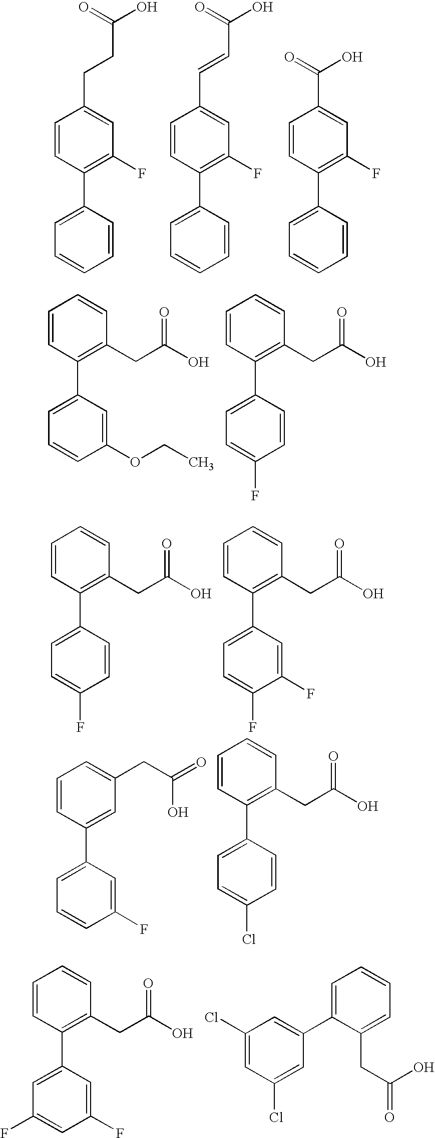

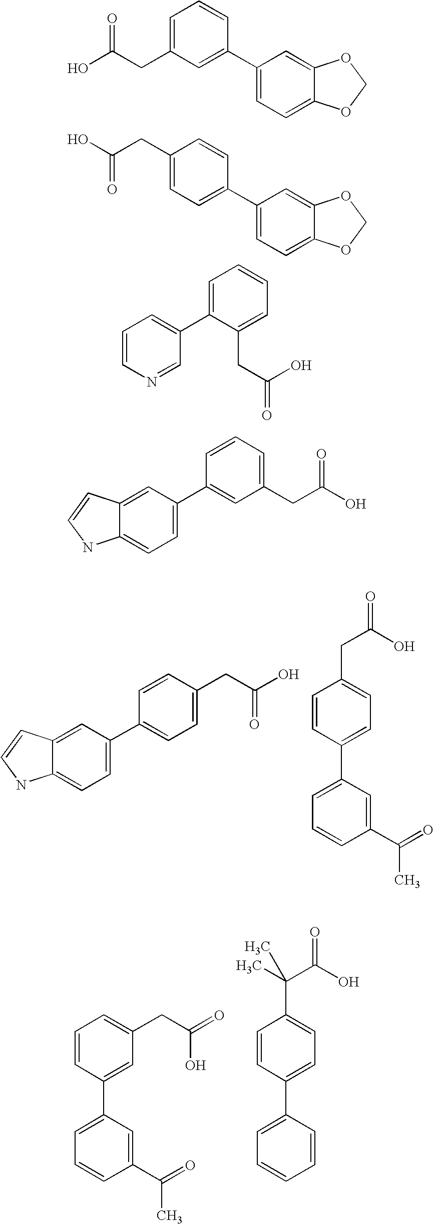

The following compounds were synthesized in an analogous manner to that described for compound 91:

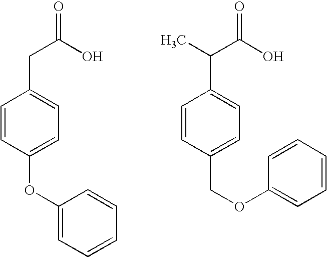

The following compounds were synthesized in an analogous manner to that described for compound 92:

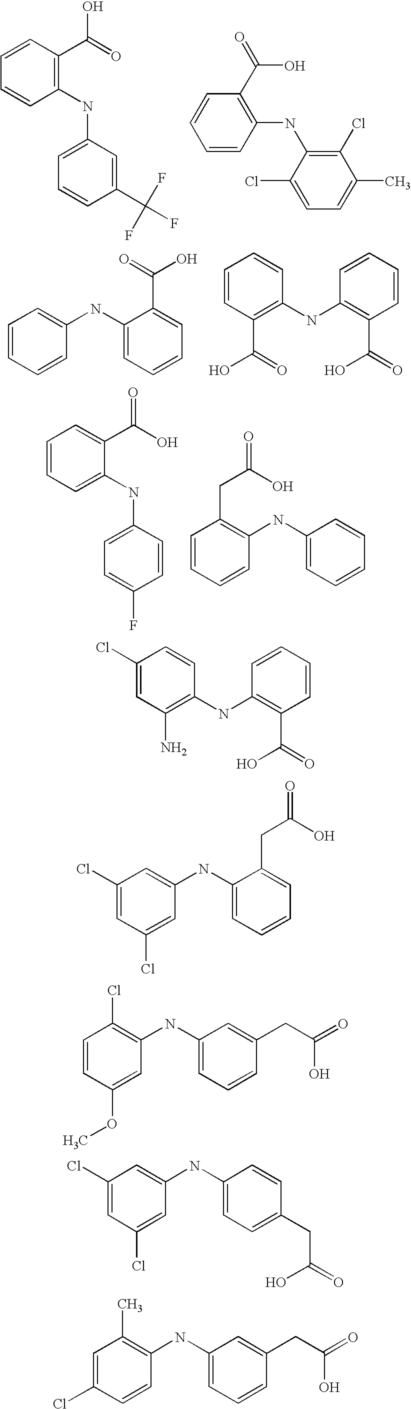

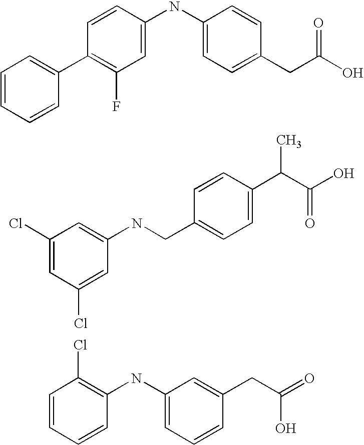

The following compounds were synthesized in an analogous manner to that described for compounds 96-111:

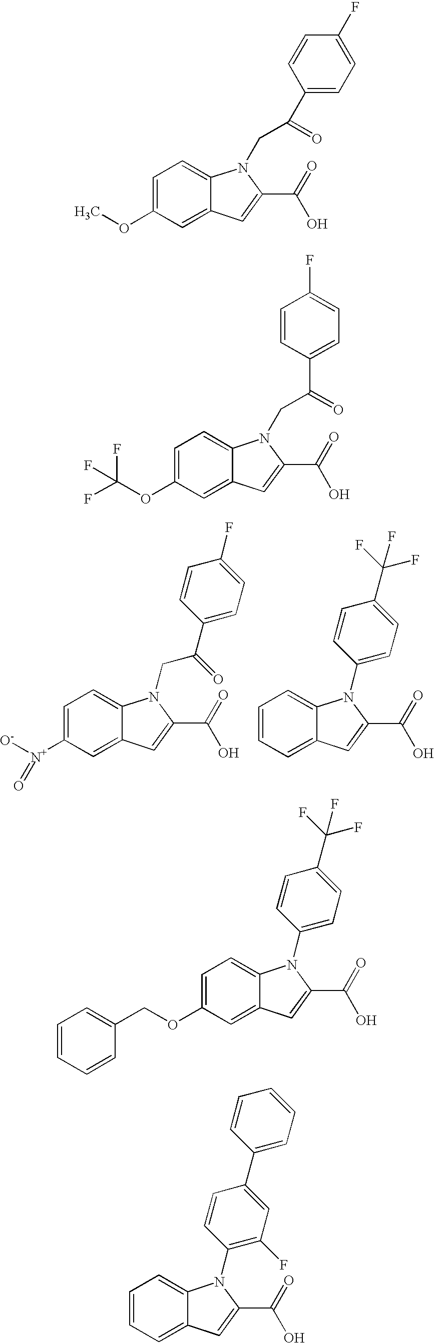

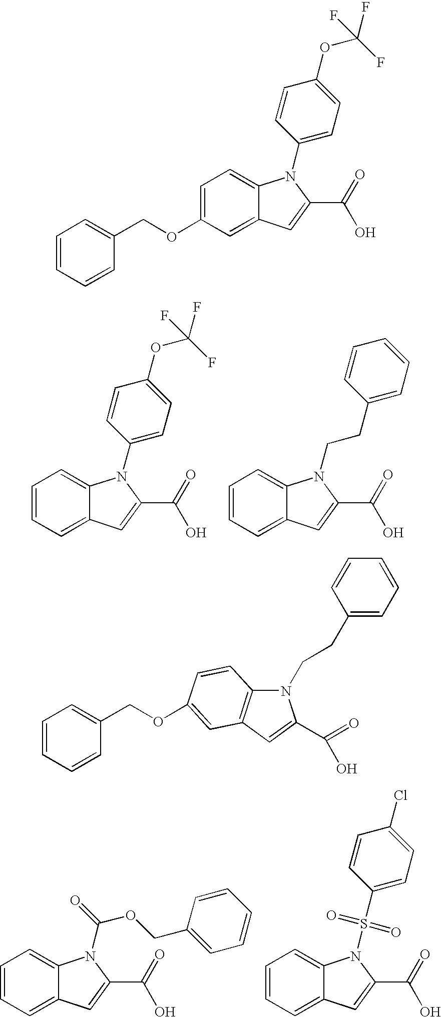

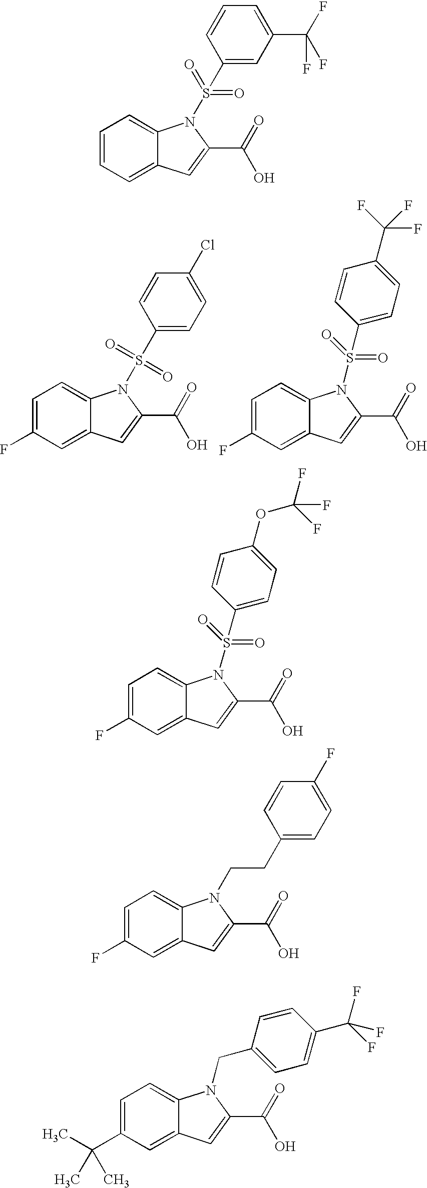

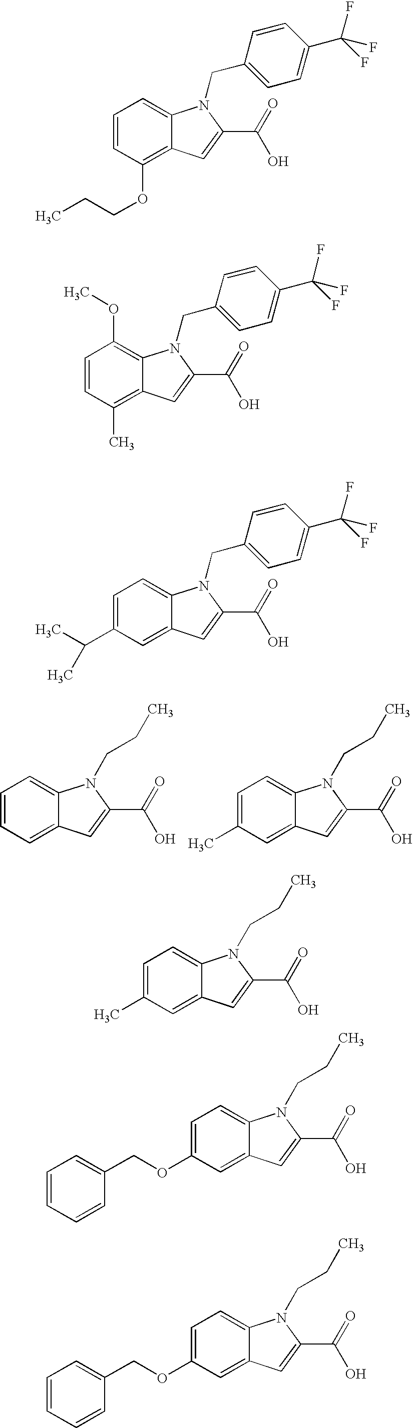

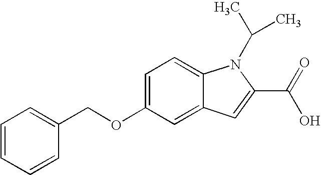

### Example 3

### Detection of Amyloid Beta with Biosource Elisa Kit (Camarillo, Calif.)

The present invention provides compositions and methods for lowering Aβ₄₂ levels. To test whether compounds and compositions are capable of modulating Aβ levels, a sandwich enzyme-linked immunosorbent assay (ELISA) is employed to measure secreted Aβ (Aβ₄₂ and/or Aβ₄₀) levels. In this example, H4 cells expressing wild type APP695 are seeded at 200,000 cells/per well in 6 well plates, and incubated at 37 degree C. with 5% CO₂ overnight. Cells are treated with 1.5 ml medium containing vehicle (DMSO) or a test compound at 1.25 μM, 2.5 μM, 5.0 μM and 10.0 μM (as well as other concentration if desirable) concentration for 24 hours or 48 hours. The supernatant from treated cells is collected into eppendorf tubes and frozen at −80 degree C. for future analysis.

The amyloid peptide standard is reconstituted and frozen samples are thawed. The samples and standards are diluted with appropriate diluents and the plate is washed 4 times with Working Wash Buffer and patted dry on a paper towel. 100 μL per well of peptide standards, controls, and dilutions of samples to be analyzed is added. The plate is incubated for 2 hours while shaking on an orbital plate shaker at RT. The plate is then washed 4 times with Working Wash Buffer and patted dry on a paper towel. Detection Antibody Solution is poured into a reservoir and 100 μL/well of Detection Antibody Solution is immediately added to the plate. The plate is incubated at RT for 2 hours while shaking and then washed four times with Working Wash Buffer and patted dry on a paper towel. Secondary Antibody Solution is then poured into a reservoir and 100 μL/well of Secondary Antibody Solution is immediately added to the plate. The plate is incubated at RT for 2 hours with shaking, washed 5 times with Working Wash Buffer, and patted dry on a paper towel.

100 μL of stabilized chromogen is added to each well and the liquid in the wells begins to turn blue. The plate is incubated for 30 minutes at room temperature and in the dark. 100 μL of stop solution is added to each well and the plate is tapped gently to mix resulting in a change of solution color from blue to yellow. The absorbance of each well is read at 450 nm having blanked the plate reader against a chromogen blank composed of 100 μL each of stabilized chromogen and stop solution. The plate is read within 2 hours of adding the stop solution. The absorbance of the standards is plotted against the standard concentration and the concentrations of unknown samples and controls are calculated.

### Example 4

### Treatment of Alzheimer's Disease with a Compound of Formulae I-Va

The compounds of Formulae I-Va can be administered twice daily as tablets containing 400 mg of active ingredient or as a capsule containing 400 mg of the active ingredient. A higher dose can be administered to the patient in need of such treatment which can involve the patient taking e.g., a 800 mg dose of a compound of Formulae I-Va in the morning and a 800 mg dose of a compound of Formulae I-Va in the evening. Typically, for the treatment of mild-to-moderate Alzheimer's disease, an individual is diagnosed by a doctor as having the disease using a suitable combination of observations. One criterion indicating a likelihood of mild-to-moderate Alzheimer's disease is a score of about 15 to about 26 on the MMSE test. Another criteria indicating mild-to-moderate Alzheimer's disease is a decline in cognitive function. Compounds of Formulae I-Va can also be administered in liquid dosage forms. The dosages can also be divided or modified, and taken with or without food. For example, the 400 mg dose can be divided into two 200 mg tablets or capsules.

Depending on the stage of the disease, the compound (i.e., Formulae I-Va) can also be administered twice daily in liquid, capsule, or tablet dosage forms where the dose has various amounts (i.e., 850 mg, 750 mg, 700 mg, 650 mg, 600 mg, 550 mg, 500 mg, 450 mg, 350 mg, 300 mg, 250 mg, 200 mg, 150 mg, and 100 mg). Again, the dosages can also be divided or modified, and taken with or without food. The doses can be taken during treatment with other medications for treating Alzheimer's disease or symptoms thereof. For example, the compound can be administered in the morning as a tablet containing 400 mg of active ingredient (i.e., a compound of Formulae I-Va) and an acetylcholine esterase inhibitor (i.e., tacrine (Cognex®), donepezil (Aricept®), rivastigmine (Exelon®), and galantamine (Reminyl®)), and/or an NMDA antagonist (i.e., memantine). It may be desirable to lower the amount of acetylcholine esterase inhibitor (and/or NMDA antagonist) and/or NSAID to avoid adverse side effects associated with higher doses of these compounds. Alternatively, the acetylcholine esterase inhibitor (and/or NMDA antagonist) and NSAID can be co-formulated into a single dosage form, i.e., liquid, tablet, capsule, etc.

Patients having mild-to-moderate Alzheimer's disease undergoing the treatment regimen of this example with a compound of Formulae I-Va in doses of about 20 mg to 1600 mg per day can experience a lessening in decline of cognitive function (as measured by the ADAS-cog or CDR sum of boxes), plaque pathology, and/or biochemical disease marker progression.

### Example 5

### Formulations

| Ingredient | Amount | Preferred Ranges |
|---|---|---|
| Compound of Formulae I-Va | 400 mg | +50% to −50% |
| Microcrystalline Cellulose | 392 mg | +50% to −50% |
| Colloidal Silicon Dioxide | 4 mg | +50% to −50% |
| Magnesium Stearate | 4 mg | +50% to −50% |

The tablets are prepared using art known procedures.

### Coated Tablets

| Ingredient | Amount | Preferred Ranges |
|---|---|---|
| Compound of Formulae I-Va | 400 mg | +50% to −50% |
| Microcrystalline Cellulose | 392 mg | +50% to −50% |
| Colloidal Silicon Dioxide | 4 mg | +50% to −50% |
| Magnesium Stearate | 4 mg | +50% to −50% |
| Coated with | | |
| Lactose monohydrate | | |
| Hydroxyl propyl methyl | | |
| cellulose | | |
| Titanium dioxide | | |
| Tracetin/glycerol triacetate | | |
| Iron oxide | | |

The coated tablets are produced using art known procedures.

### Capsules

| Ingredient | Amount | Preferred Ranges |
|---|---|---|
| Compound of Formulae I-Va | 400 mg | +50% to −50% |
| Microcrystalline Cellulose | 392 mg | +50% to −50% |
| Colloidal Silicon Dioxide | 4 mg | +50% to −50% |
| Magnesium Stearate | 4 mg | +50% to −50% |
| Encapsulated in gelatin | | |

The capsules are produced using art known procedures.

### Tablets

| Ingredient | Amount | Preferred Ranges |
|---|---|---|
| Compound of Formulae I-Va | 200 mg | +50% to −50% |
| Microcrystalline Cellulose | 196 mg | +50% to −50% |
| Colloidal Silicon Dioxide | 2 mg | +50% to −50% |
| Magnesium Stearate | 2 mg | +50% to −50% |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A compound of the formula 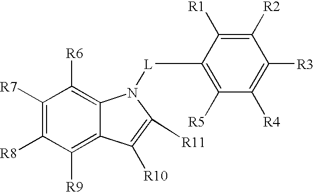
or a pharmaceutically acceptable salt thereof,
wherein L is —C(═O)— or —CH₂—;
R1, R2, R4, R5, R6, R7, and R9 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂;
R3 is selected from the group consisting of —CHF₂, —CF₃, —OCF₃, —OCHF₂, and preferably —CF₃ or —OCF₃;
R8 is H; halo (e.g., F, Cl, Br, I); C₁₋₆ alkyl (e.g., preferably methyl, ethyl, propyl, isopropyl, or —C(CH₃)₃); C₁₋₆ haloalkyl (e.g., CHF₂, CF₃); or C₂₋₆ alkoxy optionally substituted with 1, 2, 3, and 4-6 halo (e.g., F, Cl, Br, I), preferably ethoxy, propyloxy and isopropyloxy;
R10 is —R^{L}—COOH, wherein R^{L} is selected from C₁₋₆ alkyl, C₂₋₆ alkenyl and C₂₋₆ alkynyl, preferably —CH₂—; and
R11 is a C₁₋₃ alkyl (e.g., methyl, ethyl, propyl, isopropyl), preferably methyl.

2. A compound of the formula 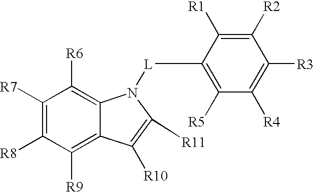
or a pharmaceutically acceptable salt thereof,
wherein L is —C(═O)—;
R1, R2, R4, R5, R6, R7, and R9 are independently H; halo (e.g., F, Cl, Br, I); C₁₋₃ alkyl; C₁₋₃ haloalkyl (e.g., CHF₂, CF₃); or C₁₋₃ alkoxy optionally substituted with 1, 2, 3, or 4 halo (e.g., F, Cl, Br, I), preferably —OCF₃, —OCHF₂; Preferably, R1, R2, R4, R5, R6, R7, and R9 are independently H or halo or methyl;
R3 is —OCF₃;
R8 is H; F, Cl or Br; C₁₋₆ alkyl (e.g., preferably methyl, ethyl, propyl, isopropyl, or —C(CH₃)₃); C₁₋₆ haloalkyl (e.g., CHF₂, CF₃); or C₂₋₆ alkoxy optionally substituted with 1, 2, 3, and 4-6 halo (e.g., F, Cl, Br, I), preferably ethoxy, propyloxy and isopropyloxy;
R10 is —CH₂COOH; and
R11 is a C₁₋₃ alkyl (e.g., methyl, ethyl, propyl, isopropyl), preferably methyl.

3. A compound of the formula 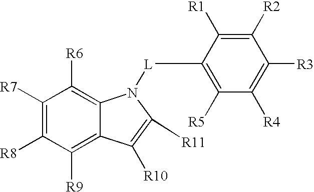
wherein L is —CH₂—;
R1, R2, R4, R5, R6, R7, and R9 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂;
R3 is —CF₃;
R8 is H; halo (e.g., F, Cl, Br, I); C₁₋₆ alkyl (e.g., preferably methyl, ethyl, propyl, isopropyl, or —C(CH₃)₃); C₁₋₆ haloalkyl (e.g., CHF₂, CF₃); or C₂₋₆ alkoxy optionally substituted with 1, 2, 3, and 4-6 halo (e.g., F, Cl, Br, I), preferably ethoxy, propyloxy and isopropyloxy; Preferably R8 is C₁₋₄ alkyl (e.g., methyl, ethyl, propyl, isopropyl, or —C(CH₃)₃);
R10 is —CH₂COOH; and
R11 is a C₁₋₃ alkyl (e.g., methyl, ethyl, propyl, isopropyl), preferably methyl.

4. A compound of the formula 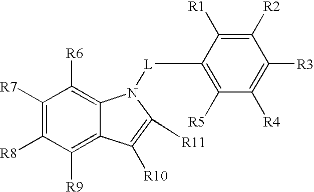
or a pharmaceutically acceptable salt thereof,
wherein L is —CH₂— or —CH(C₁₋₆ alkyl)-, and preferably —CH₂—;
R1, R2, R4, R5, R6, R7, R9 and R10 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂; or C₁₋₆ alkyl-S— optionally substituted with 1, 2, 3, and 4-6 halo (e.g., F, Cl, Br, I), preferably —SCF₃;
R3 is selected from the group consisting of C₁₋₃ haloalkyl (e.g., —CHF₂, —CF₃), —SCF₃, C₁₋₃ alkoxy, or C₁₋₃ haloalkoxy (e.g., —OCF₃, —OCHF₂), wherein optionally R₃ forms a 5 or 6-membered heterocycle with the adjacent R2 or R4 group;
R8 is H; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably ethoxy, propyloxy and isopropyloxy; C₁₋₆ alkyl-S— optionally substituted with 1, 2, 3, and 4-6 halo (e.g., F, Cl, Br, I); or —S(O)₂—(C₁₋₆ alkyl); —NO₂;
R11 is selected from the group consisting of —R^{L}—C(═O)R₄₂, —R^{L}—C(═S)R₄₂, —R^{L}—C(═O)S—R₄₃, —R^{L}—C(═O)N(R₅₂)(R₅₃), —S(O)₂—(C₁₋₆ alkyl); —R^{L}-phosphono, and —R^{L}-tetrazolyl;
R^{L} is selected from a bond, C₁₋₆ alkyl, C₂₋₆ alkenyl and C₂₋₆ alkynyl, preferably a bond or C₁ alkyl;
R₄₂ is selected from H, —OH, C₁₋₆ alkyl, C₂₋₆ alkenyl, C₂₋₆ alkynyl, C₁₋₆ alkoxy, C₂₋₆ alkenyloxy, C₂₋₆ alkynyloxy, and C₁₋₆ alkylthiol, wherein R₄₂ is optionally substituted with from one to three substituents independently selected from halo, N₃, nitro, hydroxy, thiol, CN and C₁₋₆ alkyl;
R₄₃ is H, C₁₋₆ alkyl, C₂₋₆ alkenyl or C₂₋₆ alkynyl, wherein R₄₃ is optionally substituted with from one to three substituents independently selected from halo, N₃, nitro, hydroxy, thiol, CN and C₁₋₆ alkyl; and
R₅₂ and R₅₃ are independently H, OH(R₅₂ and R₅₃ are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, or R₅₂ and R₅₃ together with the nitrogen atom to which they are both linked form a 3, 4, 5 or 6-membered heterocycle (e.g., piperidinyl, pyrrolidinyl, and morpholinyl), wherein R₅₂ and R₅₃ each is optionally substituted with 1-3 substituents wherein each substituent is independently halo, N₃, nitro, hydroxy, thiol, CN, C₁₋₆ alkyl, C₁₋₆ haloalkyl, C₁₋₆ alkoxy, —C(═O)N(R₅₄)(R₅₅), R₄₄C(═O)— or —N(R₅₄)(R₅₅), wherein R₅₄ and R₅₅ are independently H, OH or C₁₋₄ alkyl, and wherein R₄₄ is H or C₁₋₄ alkyl.

5. A compound of the formula 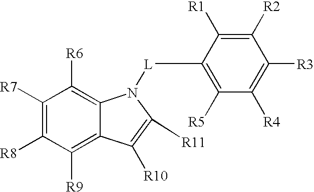
or a pharmaceutically acceptable salt thereof,
wherein L is —CH₂—;
R1, R2, R4, R5, R6, R7, R9 and R10 are independently H; OH; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably —OCF₃, —OCHF₂; or C₁₋₆ alkyl-S— optionally substituted with 1, 2, 3, and 4-6 halo (e.g., F, Cl, Br, I), preferably —SCF₃;
R3 is selected from the group consisting of —CHF₂, —CF₃, —OCF₃, or —OCHF₂;
R8 is H; halo (e.g., F, Cl, Br, I); C₁₋₆ alkyl; C₁₋₆ haloalkyl (e.g., CHF₂, CF₃); C₁₋₆ alkoxy optionally substituted with 1, 2, 3, and 4-6 halo (e.g., F, Cl, Br, I), preferably ethoxy, propyloxy and isopropyloxy; C₁₋₆ alkyl-S— optionally substituted with 1, 2, 3, and 4-6 halo (e.g., F, Cl, Br, I); or —S(O)₂—C₁₋₆ alkyl); —NO₂;
R11 is selected from the group consisting of —R^{L}—COOH; and
R^{L} is selected from a bond, C₁₋₆ alkyl, C₂₋₆ alkenyl and C₂₋₆ alkynyl, preferably a bond.

6. A method of reducing Aβ₄₂ production or secretion in a mammalian cell, comprising administering to the cell a compound according to anyone of claims 1-5.

7. Use of the compound according to anyone of claims 1-5 in the manufacture of a medicament useful in treating a disease amenable to reduction of cellular Aβ₄₂ production or secretion.

8. The use of claim 7, wherein said medicament is used in treating a neurodegenerative disorder selected from the group consisting of dementia, Alzheimer's disease, MCI, Parkinson's disease, Down's syndrome, and tauopathies (corticobasal degeneration, and progressive supranuclear palsy).

9. The use of claim 7, wherein said medicament is used in treating inclusion body myositis.
